(19) United States
(12) Patent Application Publication
(10) Pub. No.: US 2021/0000341 A1
(43) Pub. Date: Jan. 7, 2021

(54) METHOD OF DETECTING AND/OR PREDICTING SEIZURES

(71) Applicant: Children's Hospital & Research Center at Oakland, Okland, CA (US)

(72) Inventor: Rachel Kuperman, Oakland, CA (US)

(21) Appl. No.: 16/977,006

(22) PCT Filed: Feb. 28, 2019
(86) PCT No.: PCT/US2019/020116

§ 371 Date: Aug. 31, 2020

Related U.S. Application Data
(60) Provisional application No. 62/640,978, filed on Mar. 9, 2018.

(51) Int. Cl. A61B 3/113; A61B 3/11; A61B 3/14; A61N 1/36; A61B 5/00

(52) U.S. Cl. CPC A61B 3/113; A61B 3/112; A61B 5/4094; A61N 1/36053; A61N 1/36064; A61B 3/145

(57) ABSTRACT

The methods and systems described herein provide a novel approach for detecting and/or predicting an epileptic event in a subject with or without performing an EEG on the subject. Methods of identifying and treating epilepsy in a subject are also provided herein. A broad regression analysis using a lower order statistical analysis and/or a higher order statistical analysis of one or more oculometric parameters in a time series can be used to determine that the distribution of an oculometric parameter over time and/or the related dependencies of frequencies of two or more oculometric parameters over time correlate with an epileptic event. The methods and systems described herein may also be applied to one or more facial biometrics of the subject.

This application claims the benefit of U.S. Provisional Application No. 62/640,978, filed Mar. 9, 2018, which application is incorporated herein by reference in its entirety.

### INTRODUCTION

Epilepsy is a debilitating unpredictable chronic disease. Patients with epilepsy suffer from unobserved seizures during sleep and during activities where a seizure may be dangerous, such as driving. There is also a risk of sudden unexpected death in epilepsy (SUDEP). Patient autonomy and decision making are limited by the difficulty of accurately measuring seizure burden, treatment success, or excess sedation. Seizure frequency is difficult to measure because of the subtle manifestations of some seizure types and the brain's inability to remember seizures originating from certain regions. Currently, devices such as the vagal nerve stimulator (VNS) and medications can only intervene when the clinical symptoms are observed, thus frequently delaying intervention when it would be more effective earlier. Current methods to detect and/or predict seizures include clinical observation and electroencephalogram (EEG) and are the only available reliable standards to detect seizures. Despite overt clinical manifestations, patient seizure counts often fail to provide valid information as patients and parent observers fail to report between 50-55% of all recorded seizures in a monitored setting. Performing and interpreting an EEG is time and labor intensive and as a result, EEG placement is geographically limited to specialized centers and further limited to normal business hours. The present disclosure addresses the above issues and provides related advantages.

### SUMMARY

The methods and systems described herein provide a novel approach for detecting and/or predicting an epileptic event in a subject with or without performing an EEG on the subject. Methods of identifying and treating epilepsy in a subject are also provided herein. Epileptic events have a unique signature of ocular changes that currently available measuring devices are capable of measuring. A broad regression analysis using a lower order statistical analysis and/or a higher order statistical analysis of one or more oculometric parameters in a time series can be used to determine that the distribution of an oculometric parameter over time and/or the related dependencies of frequencies of two or more oculometric parameters over time correlate with an epileptic event. The methods and systems described herein may also be applied to one or more facial biometrics of the subject.

In exemplary embodiments, the disclosed methods of detecting and/or predicting an epileptic event in a subject include measuring a change in one or more oculometric parameters of at least one eye of the subject over time using a measuring device to obtain oculometric data from the subject; performing a first order statistical analysis and/or second order statistical analysis of the oculometric data; determining the presence or absence of a change relative to baseline in the first order statistical analysis and/or second order statistical analysis of the oculometric data; and indicating that an epileptic event has been detected and/or predicted when the determining indicates the presence or absence of a change in the first order statistical analysis and/or second order statistical analysis relative to baseline. Detecting and/or predicting an epileptic event in a subject as described herein may be performed without measuring at least one electroencephalogram signal of the subject.

In some embodiments, the disclosed methods of identifying and treating epilepsy in a subject include measuring a change in one or more oculometric parameters of at least one eye of the subject overtime using a measuring device to obtain oculometric data from the subject; performing a first order statistical analysis and/or second order statistical analysis of the oculometric data; determining the presence or absence of a change relative to baseline in the first order statistical analysis and/or second order statistical analysis of the oculometric data; identifying the subject as having an epileptic event and/or as at risk of an epileptic event when the determining indicates the presence or absence of a change in the first order statistical analysis and/or second order statistical analysis of the oculometric data relative to baseline; and administering an effective amount of an anti-epileptic drug to the subject identified as having an epileptic event and/or as at risk of an epileptic event. Identifying and treating epilepsy in a subject as described herein may be performed without measuring at least one electroencephalogram signal of the subject.

As a variation of the above method, the disclosed methods of identifying and treating epilepsy in a subject include measuring a change in one or more oculometric parameters of at least one eye of the subject over time using a measuring device to obtain oculometric data from the subject; performing a first order statistical analysis and/or second order statistical analysis of the oculometric data; determining the presence or absence of a change relative to baseline in the first order statistical analysis and/or second order statistical analysis of the oculometric data; identifying the subject as having an epileptic event and/or as at risk of an epileptic event when the determining indicates the presence or absence of a change in the first order statistical analysis and/or second order statistical analysis of the oculometric data relative to baseline; and transmitting an electric current through the neck of the subject identified as having an epileptic event and/or as at risk of an epileptic event to a vagus nerve in the subject, wherein the electric current is sufficient to terminate the epileptic event.

In some embodiments, the disclosed methods of detecting and/or predicting an epileptic event in a subject include measuring left and right eye movements over time using a measuring device to obtain eye movement data from the subject; identifying the presence or absence of an increase in the correlation of left and right eye movements over time based on the measuring; and indicating that an epileptic seizure has been detected and/or predicted when the identifying indicates the presence of an increase in the correlation of left and right eye movements over time.

In some embodiments, the disclosed systems of detecting and/or predicting an epileptic event in a subject include a measuring device configured to measure a change in one or more oculometric parameters of at least one eye of the subject over time; a processor unit; a non-transitory computer-readable storage medium comprising instructions, which when executed by the processor unit, cause the processor unit to perform a first order statistical analysis and/or second order statistical analysis of the oculometric data and determine the presence or absence of a change relative to baseline in the first order statistical analysis and/or second order statistical analysis of the oculometric data; and an output device configured to indicate that an epileptic event has been detected and/or predicted when a change in the first order statistical analysis and/or second order statistical analysis is determined to be present.

In some embodiments, the one or more oculometric parameters may include eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep. In some embodiments, the measuring includes measuring a change in two or more of the oculometric parameters. In some embodiments, the one or more oculometric parameters or two or more oculometric parameters include eye eccentricity, where eye eccentricity is a function of visible x-width and y-width of the pupil of an eye. In certain embodiments, the one or more oculometric parameters or two or more oculometric parameters include pupil eccentricity. In some embodiments, the one or more oculometric parameters or two or more oculometric parameters include left eye movements and right eye movements.

In some embodiments, the first order statistical analysis of the oculometric data includes performing multiple regression analysis and mean calculations. For example, in some embodiments, performing the first order statistical analysis of the oculometric data includes performing multiple regression analysis of the oculometric data. In some embodiments, the second order statistical analysis of the oculometric data includes performing variance calculations. For example, in some embodiments, performing the second order statistical analysis of the oculometric data includes performing variance calculations of the oculometric data. In some embodiments, determining the presence or absence of a change in the first order statistical analysis and/or second order statistical analysis of the oculometric data includes determining the presence or absence of an increased correlation of one or more oculometric parameters with the epileptic event. In some embodiments, determining the presence or absence of an increased correlation of one or more oculometric parameters with the epileptic event comprises determining the presence or absence of an increased correlation of eye eccentricity with the epileptic event.

In some embodiments, the disclosed methods include performing a higher order statistical analysis of the oculometric data. In some embodiments, the higher order statistical analysis of the oculometric data includes kurtosis. The disclosed methods may further include determining the presence or absence of a change relative to baseline in the higher order statistical analysis of the oculometric data such as determining the presence of a change from frequency independence to inter-frequency dependence of the oculometric data, determining the presence of a change of synchronization of the oculometric data, or determining the presence of positive excess kurtosis of the oculometric data. In other embodiments, determining the presence of positive excess kurtosis of the oculometric data includes determining the presence of positive excess kurtosis of eye eccentricity. In some embodiments, the determining step utilizes machine learning.

As a variation of the above methods, the disclosed methods of detecting and/or predicting an epileptic event in a subject may include measuring a change in one or more facial biometrics of the subject to provide facial biometrics data. In some embodiments, the disclosed methods further include performing a first order statistical analysis, a second order statistical analysis, and/or higher order statistical analysis of the facial biometrics data. In some embodiments, the disclosed methods further include determining the presence or absence of a change relative to baseline in the first order statistical analysis, a second order statistical analysis, and/or higher order statistical analysis of the facial biometrics data. In some aspects, the one or more facial biometrics includes distance between the eyes; distance between the eyelids; width of the nose; center of the nose; depth of the eye sockets; shape of the cheekbones; length of the jawline; distance between the mouth edges; center of the mouth; and/or focal weakness. In certain embodiments, the one or more facial biometrics includes mouth movements.

In some embodiments, the disclosed methods of detecting and/or predicting an epileptic event in a subject may further include measuring prodromal changes of the oculometric data and/or facial biometric data. In some embodiments, the disclosed methods include performing a first order statistical analysis, a second order statistical analysis, and/or higher order statistical analysis of the prodromal changes of the oculometric data and/or facial biometrics data and determining the presence or absence of a change relative to baseline in the first order statistical analysis, a second order statistical analysis, and/or higher order statistical analysis of the prodromal changes of the oculometric data and/or facial biometrics data.

In some embodiments, indicating that the epileptic event has been detected and/or predicted includes providing an alert to the subject or a caregiver of the subject. In other embodiments, the indicating further includes providing a responsive neurostimulation to the subject, where the responsive neurostimulation is sufficient to reduce the effect of the epileptic event, when the epileptic event is detected and/or predicted. In some embodiments, the indicating includes transmitting an electric current through the neck of a subject for which an epileptic event has been detected and/or predicted to a vagus nerve in the subject for which an epileptic event has been detected and/or predicted, wherein the electric current is sufficient to terminate the epileptic event, when the epileptic event is detected and/or predicted or administering an effective amount of an anti-epilpetic drug to the subject, when the epileptic event is detected and/or predicted.

### Definitions

As described herein, the term “epileptic event” may refer to an epileptic seizure including generalized seizures and/or focal (or partial) seizures. Exemplary epileptic events include absence seizures, atypical absence seizures, tonic-clonic seizures, clonic seizures, tonic seizures, atonic seizures, myoclonic seizures, simple partial seizures, complex partial seizures, secondary generalized seizures, and/or infantile spasms. In some embodiments, an epileptic event may refer to a condition related to, or resulting from, an epileptic disorder, including, but not limited to, Todd's paralysis, and/or sudden unexpected death in epilepsy (SUDEP). In some embodiments, the epileptic event is an absence seizure.

As described herein, the terms “oculometric parameters” and “oculometrics” are used interchangeably to refer to autonomic changes related to the eye(s) of a subject that are collected before, during or after an epileptic event. Exemplary oculometric parameters include, but are not limited to, eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep. In some embodiments, the one or more oculometric parameters includes eye eccentricity.

As described herein, the term “eye eccentricity” generally refers to a calculated variable that is a function of the visible x width and y width of the pupil. In some embodiments, eye eccentricity is a combined variable which changes as the eyelid position, position of the sides of the eye, pupil area, and/or blink frequency change(s). Eye eccentricity can be defined by the following formula: eccentricity=c/a, where c is the distance from the center to a focus and a is the distance from that focus to a vertex. In some embodiments, eccentricity of the eye is calculated as if the eye were an approximated ellipse. In some embodiments, an ellipse is the locus of points such that the sum of the distance to each focus is constant. For example, if the pupil is positioned in the middle of an eye and the eye is wide open with the eyelid not obscuring the pupil, the pupil appears perfectly round and the x width and y width are equal, thus resulting in an eye eccentricity of zero because the eccentricity of a circle is zero. In some embodiments, an eye is deviated upward, yet still positioned in the midline of an eye, then part of the pupil is obscured by the eyelid, thus resulting in a longer measured visible x width as compared to an eyelid obscured y width. In some embodiments, an eye is deviated to the far left, where the eyelid obscures part of the pupil, thus resulting in a longer y width measurement as compared to the x width measurement. In exemplary embodiments, eye eccentricity combines multiple variables.

As described herein, the term “first order statistical analysis” refers to a lower order statistical analysis involving moments and cumulants of a first order. For example, a first order statistical analysis includes a breakdown of frequencies present in each oculometric parameter over time. A first order statistical analysis may be used to determine if the absence or presence of certain frequencies of oculometric data and/or facial biometrics data correlates with epileptic events. Frequencies or repetition rates for each dependent variable are considered as independent variables. In some embodiments, the first order statistical analysis of the oculometric data and/or facial biometrics data includes multiple regression analysis and/or mean calculations. First order statistics may be calculated linearly having a power of 1.

As described herein, the term “second order statistical analysis” refers to a lower order statistical analysis involving moments and cumulants of a second order. In some embodiments, the second order statistical analysis of the oculometric data and/or facial biometrics data includes variance calculations. “Variance” means the expectation of the squared deviation of a random variable from its mean. Second order statistics may be calculated quadratically having a power of 2.

As described herein, the term “higher order statistical analysis” refers to moments and cumulants of a third order and beyond. For instance, higher order analysis may include determining a change in synchronization including frequency synchronization, e.g., dependent frequencies and/or uncoupled frequencies, of oculometric data and/or facial biometrics data over a time series as it relates to an epileptic event, which is not revealed in a first order statistical analysis and/or second order statistical analysis. Determining a change in synchronization may occur before, during, or after the occurrence of an epileptic event. Frequencies or repetition rates of an originally independent variable may become dependent. For example, in some embodiments, a mechanism relates the frequency of pupil dilation to the frequency of mouth edge movements, thus creating an intrinsic dependence. In exemplary embodiments, an epileptic event may be detected and/or predicted by the occurrence of a transition from frequency independence to inter-frequency dependence.

Exemplary embodiments of higher order statistical analysis include kurtosis and skewness, which further describe the shape of a distribution. Higher order statistical analysis may include bispectral analysis, generalized linear and/or nonlinear regression analysis. Higher order statistical analysis may be performed using standard techniques known in the art, including, but not limited to, Chua et al. (2010) and Mendel (1991), the disclosures of which are incorporated herein by reference.

As described herein, the term “kurtosis” is defined by the formula

$$k = \frac{{Σ ( x - μ )}^{4}}{{σ}^{4}} ,$$

where x is the variable under test, μ is the mean, and σ is the standard deviation. Kurtosis is a dimensionless quantity. Kurtosis represents how stable one or more oculometric parameters, e.g. eye eccentricity or eye movement, appears, and describes the shape of the distribution. In some aspects, kurtosis is generally described as the degree of peakedness of a distribution. For example, a higher kurtosis relative to baseline indicates more points fall on or near the mean, and as a result, the less variable the distribution or, e.g., the less an eye is moving. The smaller the kurtosis relative to baseline indicates the more variable the distribution or, e.g., the more an eye is moving. Kurtosis describes how outlier prone a variable may be. In certain aspects, kurtosis of a normal distribution has a value of 3. In some embodiments, the baseline kurtosis varies per subject. In some embodiments, kurtosis of the oculometric data, facial biometrics data, and/or eye movement data is measured in about a 1-second to a 15-second window, inclusive, such as a 1-second to a 3-second window, a 1-second to a 4-second window, a 1-second to a 5-second window, a 1-second to a 6-second window, a 1-second to a 7-second window, an 1-second to an 8-second window, a 1-second to a 9-second window, or a 1-second to a 10-second window. In some embodiments, kurtosis measurements are performed in a 5-second window.

As described herein, the term “baseline” generally refers to an initial value measured or a known standard value for a specific oculometric parameter or facial biometric of a subject not currently experiencing an epileptic event. In some embodiments, the baseline of a subject may be measured during an interictal period between seizures when the body functions at a relatively normal level for the subject. A baseline may be subject-specific and used for comparison or a control for the subject. In some embodiments, a baseline value may be confirmed by an EEG measurement as occurring in the absence of an epileptic event.

### BRIEF DESCRIPTION OF THE DRAWINGS

The invention may be best understood from the following detailed description when read in conjunction with the accompanying drawings. Included in the drawings are the following figures:

FIGS. 1A-1C depict the analysis of oculometric data derived from a subject experiencing an epileptic event. FIG. 1A shows eye eccentricity as percent of max eccentricity per patient plotted against time for the left eye (FIG. 1A, left) and right eye (FIG. 1A, right). FIG. 1B shows kurtosis over time in the left eye (FIG. 1B, left) and right eye (FIG. 1B, right). FIG. 1C shows the cross-correlation of eccentricity between the left eye and the right eye thus depicting the in-sync behavior of the eyes during and after an epileptic event (FIG. 1C, center). Photographs of the left eye (FIG. 1C, left) and right eye (FIG. 1C, right) during the epileptic event are also provided. The vertical red bars on the graphs denote the occurrence of an epileptic event as confirmed by EEG.

FIGS. 2A-2C depict the analysis of oculometric data derived from the same subject as in FIGS. 1A-1C experiencing a different epileptic event. FIG. 2A shows eye eccentricity as percent of max plotted against time for the left eye (FIG. 2A, left) and right eye (FIG. 2A, right). FIG. 2B shows kurtosis over time in the left eye (FIG. 2B, left) and right eye (FIG. 2B, right). FIG. 2C shows the cross-correlation of eccentricity between the left eye and the right eye (FIG. 2C, center). Photographs of the left eye (FIG. 2C, left) and right eye (FIG. 2C, right) during the epileptic event are also provided. The vertical red bars on the graphs denote the occurrence of an epileptic event as confirmed by EEG.

FIGS. 3A-3C depict the analysis of oculometric data derived from the same subject as in FIGS. 1A-1C and 2A-2C having closed eyes during a different epileptic event. FIG. 3A shows eye eccentricity as percent of max plotted against time for the left eye (FIG. 3A, left) and right eye (FIG. 3A, right). FIG. 3B shows kurtosis over time in the left eye (FIG. 3B, left) and right eye (FIG. 3B, right). FIG. 3C shows the cross-correlation of eccentricity between the left eye and the right eye (FIG. 3C, center). Photographs of the closed left eye (FIG. 3C, left) and right eye (FIG. 3C, right) during the epileptic event are also provided. The vertical red bars on the graphs denote the occurrence of an epileptic event as confirmed by EEG.

FIGS. 4A-4C depict the analysis of oculometric data derived from a subject experiencing an epileptic event at the beginning of the record. FIG. 4A shows eye eccentricity as percent of max plotted against time for the left eye (FIG. 4A, left) and right eye (FIG. 4A, right). FIG. 4B shows kurtosis over time in the left eye (FIG. 4B, left) and right eye (FIG. 4B, right). FIG. 4C shows the cross-correlation of eccentricity between the left eye and the right eye (FIG. 4C, center). Photographs of the occluded left eye (FIG. 4C, left) and right eye (FIG. 4C, right) during the epileptic event are also provided. The vertical red bars on the graphs denote the occurrence of an epileptic event as confirmed by EEG.

FIGS. 5A-5C depict the analysis of oculometric data derived from the same subject as in FIGS. 4A-4C experiencing a different epileptic event. FIG. 5A shows eye eccentricity as percent of max plotted against time for the left eye (FIG. 5A, left) and right eye (FIG. 5A, right). FIG. 5B shows kurtosis over time in the left eye (FIG. 5B, left) and right eye (FIG. 5B, right). FIG. 5C shows the cross-correlation of eccentricity between the left eye and the right eye (FIG. 5C, center). Photographs of the left eye (FIG. 5C, left) and right eye (FIG. 5C, right) during the epileptic event are also provided. The vertical red bars on the graphs denote the occurrence of an epileptic event as confirmed by EEG.

FIGS. 6A-6C depict the analysis of oculometric data derived from the same subject as in FIGS. 4A-4C and 5A-5C experiencing a different epileptic event. FIG. 6A shows eye eccentricity as percent of max plotted against time for the left eye (FIG. 6A, left) and right eye (FIG. 6A, right). FIG. 6B shows kurtosis over time in the left eye (FIG. 6B, left) and right eye (FIG. 6B, right). FIG. 6C shows the cross-correlation of eccentricity between the left eye and the right eye (FIG. 6C, center). Photographs of the left eye (FIG. 6C, left) and right eye (FIG. 6C, right) during the epileptic event are also provided. The vertical red bars on the graphs denote the occurrence of an epileptic event as confirmed by EEG.

FIGS. 7A-7C depict the analysis of oculometric data derived from the same subject as in FIGS. 4A-4C, 5A-5C, and 6A-6C having closed eyes during a different epileptic event. FIG. 7A shows eye eccentricity as percent of max plotted against time for the left eye (FIG. 7A, left) and right eye (FIG. 7A, right). FIG. 7B shows kurtosis over time in the left eye (FIG. 7B, left) and right eye (FIG. 7B, right). FIG. 7C shows the cross-correlation of eccentricity between the left eye and the right eye (FIG. 7C, center). Photographs of the closed left eye (FIG. 7C, left) and right eye (FIG. 7C, right) during the epileptic event are also provided. The vertical red bars on the graphs denote the occurrence of an epileptic event as confirmed by EEG.

FIGS. 8A-8C depict the analysis of oculometric data derived from the same subject as in FIGS. 4A-4C, 5A-5C, 6A-6C, and 7A-7C having closed eyes during a different epileptic event. FIG. 8A shows eye eccentricity as percent of max plotted against time for the left eye (FIG. 8A, left) and right eye (FIG. 8A, right). FIG. 8B shows kurtosis over time in the left eye (FIG. 8B, left) and right eye (FIG. 8B, right). FIG. 8C shows the cross-correlation of eccentricity between the left eye and the right eye (FIG. 8C, center). Photographs of the closed left eye (FIG. 8C, left) and right eye (FIG. 8C, right) during the epileptic event are also provided. The vertical red bars on the graphs denote the occurrence of an epileptic event as confirmed by EEG.

FIGS. 9A-9C depict the analysis of oculometric data derived from a subject experiencing two epileptic events within 30 seconds of each other and focuses specifically on the first epileptic event experienced. FIG. 9A shows eye eccentricity as percent of max plotted against time for the left eye (FIG. 9A, left) and right eye (FIG. 9A, right). FIG. 9B shows kurtosis over time in the left eye (FIG. 9B, left) and right eye (FIG. 9B, right). FIG. 9C shows the cross-correlation of eccentricity between the left eye and the right eye (FIG. 9C, center). Photographs of the left eye (FIG. 9C, left) and right eye (FIG. 9C, right) during the epileptic event are also provided. The vertical red bars on the graphs denote the occurrence of an epileptic event as confirmed by EEG.

FIGS. 10A-10C depict the analysis of oculometric data derived from the same subject as in FIGS. 9A-9C focusing specifically on the second epileptic event experienced in a 30 second-window. FIG. 10A shows eye eccentricity as percent of max plotted against time for the left eye (FIG. 10A, left) and right eye (FIG. 10A, right). FIG. 10B shows kurtosis over time in the left eye (FIG. 10B, left) and right eye (FIG. 10B, right). FIG. 10C shows the cross-correlation of eccentricity between the left eye and the right eye (FIG. 10C, center). Photographs of the left eye (FIG. 10C, left) and right eye (FIG. 10C, right) during the epileptic event are also provided. The vertical red bars on the graphs denote the occurrence of an epileptic event as confirmed by EEG.

### DETAILED DESCRIPTION

The methods and systems described herein provide a novel approach for detecting and/or predicting an epileptic event in a subject including measuring a change in one or more oculometric parameters, e.g., eye eccentricity, and/or facial biometric parameters, e.g., distance between the eyes, over time using a measuring device to obtain oculometric data and/or facial biometric data from the subject; performing a first order statistical analysis, e.g., multiple regression analysis, second order statistical analysis, e.g., variance, and/or a higher order statistical analysis, e.g., kurtosis, of the oculometric data and/or facial biometric data; determining the presence or absence of a change relative to baseline in the first order statistical analysis, the second order statistical analysis, and/or the higher order statistical analysis of the oculometric data and/or facial biometric data; and indicating that an epileptic event has been detected and/or predicted when the determining indicates the presence or absence of a change in the first order statistical analysis, the second order statistical analysis, and/or higher order statistical analysis relative to baseline. Epileptic events have a unique signature of ocular changes that currently available measuring devices are capable of measuring, e.g., Eye-Com Biosensor™ Model EC-7T or Pupil Labs Pupi™. A broad regression analysis using a lower order statistical analysis and/or higher order statistical analysis of one or more oculometric parameters and/or facial biometric parameters in a time series can be used to determine that the distribution of an oculometric parameter and/or facial biometric parameter over time, and/or the related dependencies of frequencies of two or more oculometric parameters and/or facial biometric parameters over time correlate with an epileptic event.

The methods described herein further provide an approach of identifying and treating epilepsy in a subject including measuring a change in one or more oculometric parameters of at least one eye and/or one or more facial biometrics of the subject over time using a measuring device to obtain oculometric data and/or facial biometrics data from the subject; performing a first order statistical analysis, a second order statistical analysis, and/or a higher order statistical analysis of the oculometric data and/or facial biometrics data; determining the presence or absence of a change relative to baseline in the first order statistical analysis, the second order statistical analysis, and/or higher order statistical analysis of the oculometric data and/or facial biometrics data; identifying the subject as having an epileptic event and/or as at risk of an epileptic event when the determining indicates the presence or absence of a change in the first order statistical analysis, the second order statistical analysis, and/or higher order statistical analysis of the oculometric data relative to baseline; and administering an effective amount of an anti-epileptic drug to the subject identified as having an epileptic event and/or as at risk of an epileptic event. In other embodiments, the disclosed methods include transmitting an electric current through the neck of the subject identified as having an epileptic event and/or as at risk of an epileptic event to a vagus nerve in the subject, wherein the electric current is sufficient to terminate the epileptic event.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already afflicted (e.g., those having epileptic events) as well as those in which prevention is desired (e.g., those with increased susceptibility to having an epileptic event; those suspected of having an epileptic event; those having one or more risk factors for an epileptic event, etc.).

The terms “individual”, “subject”, “host”, and “patient”, are used interchangeably herein and refer to any mammalian subject for whom diagnosis, treatment, or therapy is desired, such as humans. “Mammal” for purposes of treatment refers to any animal classified as a mammal, including humans, domestic and farm animals, and zoo, sports, or pet animals, such as non-human primates, dogs, horses, cats, cows, sheep, goats, pigs, camels, etc. In some cases, the mammal is a human.

An “effective amount” means the amount of a compound that, when administered to a mammal or other subject for treating a disease, is sufficient, in combination with another agent, or alone in one or more doses, to effect such treatment for the disease. The “effective amount” will vary depending on the compound, the disease and its severity and the age, weight, etc., of the subject to be treated. For example, an effective amount of an anti-epileptic drug may be an amount that reduces and/or eliminates the physiological effects and/or symptoms and/or frequency of epileptic seizure in a subject.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “an epileptic event” includes a plurality of such epileptic events and reference to “an oculometric parameter” includes reference to one or more oculometric parameters and equivalents thereof known to those skilled in the art, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, described herein are a variety of additional methods and applications, which may be performed in connection with the methods described herein relating to detecting and/or predicting an epileptic event in a subject or diagnosing and treating epilepsy in a subject. In this regard it is considered that any of the non-limiting aspects of the disclosure numbered 1-282 herein may be modified as appropriate with one or more steps of such methods and applications, and/or that such methods and applications may detect and/or predict an epileptic event of a subject according to one or more of the non-limiting aspects of the disclosure numbered 1-282 herein. Such methods and applications include, without limitation, those described in the sections herein, entitled: Methods; Epileptic Events; Oculometric Parameters; Facial Biometrics; Prodromal Changes; Lower Order Statistical Analysis; Higher Order Statistical Analysis; Cross-correlation; Synchronization; Machine Learning; Alerts; Pharmaceutical Treatments; Subjects Suitable for Treatment; Systems; Measuring Devices; Processor Units; and Output Devices.

### Methods

As summarized above, the methods and systems described herein provide a novel approach of detecting and/or predicting an epileptic event in a subject with or without performing an EEG on the subject. Methods of identifying and treating epilepsy in a subject are also provided herein. Epileptic events have a unique signature of ocular changes that currently available measuring devices are capable of measuring. A broad regression analysis using a lower order statistical analysis and/or higher order statistical analysis of one or more oculometric parameters in a time series can be used to determine that the distribution of an oculometric parameter over time and/or the related dependencies of frequencies of two or more oculometric parameters over time correlate with an epileptic event. The methods and systems described herein may also be applied to one or more facial biometrics of the subject.

As described more fully herein, in various aspects the subject methods may be used for detecting and/or predicting an epileptic event in a subject. The methods may include measuring one or more oculometric parameters in at least one eye, one or more facial biometrics, and/or left and right eye movements over time. In some embodiments, an epileptic event in a subject may be predicted about 1 second to 48 hours prior to an epileptic event, inclusive, such as 1 second to 10 minutes, 1 second to 20 minutes, 1 second to 40 minutes, 1 second to 1 hour, 1 second to 5 hours, 1 second to 10 hours, 1 second to 15 hours, 1 second to 24 hours, 1 second to 30 hours, 1 second to 35 hours, 1 second to 40 hours, or 1 second to 45 hours, inclusive.

In some aspects, the subject methods may be used for identifying and treating epilepsy in a subject. Such aspects may include administering an effective amount of an anti-epileptic drug to the subject identified as having an epileptic event and/or as at risk of an epileptic event. In other aspects, the methods include providing a responsive neurostimulation to the subject, wherein the responsive neurostimulation is sufficient to reduce the effect of the epileptic event, when the subject is identified as having an epileptic event and/or as at risk of an epileptic event. In some embodiments, the methods further include transmitting an electric current through the neck of the subject for which an epileptic event has been detected and/or predicted to a vagus nerve in the subject for which an epileptic event has been detected and/or predicted, wherein the electric current is sufficient to terminate the epileptic event, when the subject is identified as having an epileptic event and/or as at risk of an epileptic event. For example, vagus nerve stimulation (VNS) is an adjunctive therapy that has become commercially available for intractable epilepsy, as described in Uthman et al., “Vagus nerve stimulation for seizures,” Arch Med Res. 2000; 31(3): 300-3, the disclosure of which is incorporated herein by reference. See also, U.S. Pat. Nos. 6,341,236; 6,961,618 and 7,292,890, the disclosure of each of which is incorporated by reference herein.

A number of variations of these basic approaches will now be outlined in greater detail below.

### Epileptic Events

As used herein, the term “epilepsy” refers to a recurrent, paroxysmal disorder of cerebral function characterized by sudden, brief attacks of altered consciousness, motor activity, sensory phenomena, or inappropriate behavior caused by excessive discharge of cerebral neurons. Seizures result from a generalized or focal disturbance of cortical function, which may be due to various cerebral or systemic disorders. Seizures may also occur as a withdrawal symptom after long-term use of alcohol, hypnotics, or tranquilizers. In many disorders, single seizures occur. However, seizures may recur at intervals for years or indefinitely, in which case epilepsy is diagnosed. Epileptic seizures have four different states: the preictal state, which is a state that appears before the seizure begins, the ictal state that begins with the onset of the seizure and ends with an attack, the postictal state that starts after ictal state, and interictal state that starts after the postictal state of the first seizure and ends before the start of preictal state of consecutive seizure.

Manifestations of epilepsy depend on the type of seizure, which may be classified as focal/partial or generalized. In partial seizures, the excess neuronal discharge is contained within one region of the cerebral cortex. In generalized seizures, the discharge bilaterally and diffusely involves the entire cortex. A focal lesion of one part of a hemisphere may activate the entire cerebrum bilaterally so rapidly that it produces a generalized tonic-clonic seizure before a focal sign appears. Simple partial seizures consist of motor, sensory, or psychomotor phenomena without loss of consciousness. The specific phenomenon reflects the affected area of the brain. In complex partial seizures, the patient loses contact with the surroundings for 1 to 2 minutes. Mental confusion continues another 1 or 2 minutes after motor components of the attack subside. These seizures may develop at any age. Complex partial seizures most commonly originate in the temporal lobe but may originate in any lobe of the brain including the frontal lobe. Generalized seizures cause loss of consciousness and motor function from the onset. Such attacks often have a genetic or metabolic cause and may be primarily generalized (bilateral cerebral cortical involvement at onset) or secondarily generalized (local cortical onset with subsequent bilateral spread). Types of generalized seizures include infantile spasms and absence, tonic-clonic, atonic, and myoclonic seizures.

Absence seizures are characterized by brief, primarily generalized attacks manifested by a 10- to 30-second loss of consciousness and eyelid flutterings, with or without loss of axial muscle tone. Affected patients do not fall or convulse; they abruptly stop activity and resume it just as abruptly after the seizure. Absence seizures have prominent ocular manifestations as part of the seizure semiology. The ocular manifestations consist of fixation, forced deviation of the globes upward or laterally, and/or myoclonic twitches of the upper lids. Absence epilepsy typically presents between the ages of 4 to 8 with a peak between ages 6 to 7. Children typically have several dozen seizures daily which may be induced with hyperventilation.

Generalized tonic-clonic seizures typically begin with an outcry and continue with loss of consciousness and falling, followed by tonic, then clonic contractions of the muscles of the extremities, trunk, and head. Seizures usually last 1 to 2 minutes. Secondarily generalized tonic-clonic seizures begin with a simple partial or complex partial seizure. Atonic seizures are brief, primarily generalized seizures in children, characterized by complete loss of muscle tone and consciousness. The child falls or pitches to the ground, so that seizures pose the risk of serious trauma, particularly head injury. Myoclonic seizures are brief, lightning-like jerks of a limb, several limbs, or the trunk, and may be repetitive, leading to a tonic-clonic seizure. There is no loss of consciousness.

In some instances, seizures may show pulling of one side of the mouth or face, or change in expression or emotion, such as fear, or pain. After a partial seizure, Todd's paralysis may present with a change in oculometric and facial biometrics data, showing slowing of movements, decreased range of movements, and relative slackening of facial muscles.

Exemplary epileptic events that may be detected and/or predicited according to the methods described herein include, but are not limited to, absence seizures, tonic-clonic seizures, clonic seizures, tonic seizures, atonic seizures, myoclonic seizures, simple partial seizures, complex partial seizures, secondary generalized seizures, infantile spasms, and/or frontal lobe seizures. In some embodiments, an epileptic event may refer to a condition related to, or resulting from, an epileptic disorder, including, but not limited to, SUDEP and Todd's paralysis. SUDEP is a poorly understood phenomenon and one of the leading causes of death in subjects with epilepsy. In certain embodiments, the provided methods and systems may predict risk of SUDEP.

### Oculometric Parameters

As summarized above, the methods of detecting and/or predicting an epileptic event in a subject as disclosed herein include measuring a change in one or more oculometric parameters of at least one eye of the subject over time using a measuring device to obtain oculometric data from the subject; performing a first order statistical analysis, a second order statistical analysis, and/or a higher order statistical analysis of the oculometric data; determining the presence or absence of a change relative to baseline in the first order statistical analysis, second order statistical analysis, and/or higher order statistical analysis of the oculometric data; and indicating that an epileptic event has been detected and/or predicted when the determining indicates the presence or absence of a change in the first order statistical analysis, second order statistical analysis, and/or higher order statistical analysis relative to baseline. The methods of identifying and treating epilepsy in a subject as disclosed herein also include measuring a change in one or more oculometric parameters of at least one eye of the subject over time using a measuring device to obtain oculometric data from the subject. In such methods, and the related systems described herein, the one or more oculometric parameters include two or more oculometric parameters, three or more oculometric parameters, four or more oculometric parameters, five or more oculometric parameters, six or more oculometric parameters, seven or more oculometric parameters, eight or more oculometric parameters, nine or more oculometric parameters, ten or more oculometric parameters, eleven or more oculometric parameters, twelve or more oculometric parameters, thirteen or more oculometric parameters, fourteen or more oculometric parameters, fifteen or more oculometric parameters, or twenty or more oculometic parameters, e.g., as described in greater detail below. In some embodiments, the disclosed methods and systems include measuring a change in 1 to 2 oculometic parameters, 2 to 3 oculometic parameters, 3 to 4 oculometic parameters, 4 to 5 oculometic parameters, 5 to 6 oculometic parameters, 6 to 7 oculometic parameters, 7 to 8 oculometic parameters, 8 to 9 oculometic parameters, 9 to 10 oculometic parameters, 10 to 11 oculometic parameters, 11 to 12 oculometic parameters, 12 to 13 oculometic parameters, 13 to 14 oculometic parameters, 14 to 15 oculometic parameters, 15 to 16 oculometic parameters, 16 to 17 oculometic parameters, 17 to 18 oculometic parameters, 18 to 19 oculometic parameters, or 19 to 20 oculometic parameters, e.g., as described in greater detail below.

In some embodiments, the disclosed methods include measuring left and right eye movements over time using a measuring device to obtain eye movement data from the subject. In such embodiments, the disclosed methods further include identifying the presence or absence of an increase in the correlation of left and right eye movements over time based on the measuring and indicating that an epileptic seizure has been detected and/or predicted when the identifying indicates the presence of an increase in the correlation of left and right eye movements over time.

The disclosed methods and systems herein provide a novel approach of detecting and/or predicting an epileptic event in a subject with or without performing an EEG on the subject. The eyes of the subject are typically open during a seizure and can have upward gaze deviation, empty stare with no lid or eye movement, as well as eye blink rate and pupillary dilation. Other exemplary eye movements include eye eccentricity; pupil constriction rate; pupil constriction velocity; pupil dilation rate; velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink duration; and/or eye activity during sleep.

Thus, in some embodiments, the disclosed methods and systems include measuring a change in any one or more, example, any 2, 3, 4, 5 or more, of eye eccentricity; pupil constriction rate; pupil constriction velocity; pupil dilation rate; velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink duration; and/or eye activity during sleep. Such eye movements may be captured and recorded in real-time through all stages of an epileptic event (i.e. preictal period, ictal and postictal state) using a suitable measuring device. In some embodiments, the measuring device is configured to obtain oculometric data from the subject for about 30 minutes to about 60 minutes. In some embodiments, the measuring device is configured to obtain oculometric data from the subject, either continuously or intermittently, for a desired amount of minutes, hours, days, months, or years, such as about 5 minutes to 10 years, inclusive, including 5 minutes to 20 minutes, 5 minutes to 30 minutes, 5 minutes to 40 minutes, 5 minutes to 50 minutes, 5 minutes to 1 hour, 5 minutes to 10 hours, 5 minutes to 20 hours, 5 minutes to 1 day, 5 minutes to 10 days, 5 minutes to 20 days, 5 minutes to 1 month, 5 minutes to 5 months, 5 minutes to 10 months, 5 minutes to 1 year, 5 minutes to 2 years, 5 minutes to 5 years, or 5 minutes to 8 years, inclusive. In some aspects, the oculometric data from the subject is captured at about 30 frames per second (fps) or more. In other aspects, the oculometric data from the subject is captured at about 20 fps to about 400 fps, inclusive, such as 20 fps to about 60 fps, 20 fps to about 100 fps, 20 fps to about 150 fps, 20 fps to about 200 fps, 20 fps to about 300 fps, or 20 fps to about 400 fps.

The terms “ictal” and “seizure” as described herein, may be used interchangeably to mean the period of time during an epileptic cycle in which seizures occur. An epileptic cycle may be divided into three sub-cycles: ictal/seizure (e.g., partial, complex-partial, simple-partial seizure events), postictal (e.g., a time period after the ictal period, but before the patient returns to the interictal or baseline levels of function) and interictal, when the body functions of the subject are at a baseline for the subject.

Different epileptic event types may have varying oculometric and facial biometric patterns based upon which parts of the brain are involved. For example, a generalized seizure from the whole brain seizing at once may result in ocular and facial synchrony resulting in a spike in kurtosis. However, a focal seizure may drive eye movements and face movements more asymmetrically resulting in a different oculometric and face biometric pattern. In some embodiments, a relative change in in-sync movements of the right and left eyes occur after a partial seizure.

In some embodiments, oculometric and facial biometrics data may be used to identify the onset zones and aid in surgical epilepsy assessments. In some embodiments, measuring the oculometric and facial biometrics data may allow for the quantitation of previous clinical observations such as allowing for accurate localization to aid in focal epilepsy surgery. In some embodiments, oculometrics may be used to determine impaired awareness or speech during an epileptic event. Subjects are typically cognitively tested during the occurrence of an epileptic event to help determine which parts of the brain are involved. In some embodiments, oculometric data may yield feedback, e.g., reading and following commands, to allow for better understanding of impairment and localization of an epileptic event even in a subject whose event is impairing their ability to communicate.

In some embodiments, the sampling frequency may capture faster frequency eye movements such as saccades, thus producing a richer data set which can be analyzed. Exemplary oculometric parameters include, but are not limited to, eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.

In some embodiments, the one or more oculometric parameters include pupillary change such as pupil constriction rate, pupil constriction velocity, pupil dilation rate, pupil dilation velocity, hippus, x and y location of pupil, pupil rotation, pupil area to iris area ratio, and/or pupil diameter. As described herein, the term “hippus” refers to a continuous oscillation of pupillary diameter in the absence of light flux variations or other external stimuli.

In some embodiments, the one or more oculometric parameters include eyelid movement such as rapid or slow, rhythmic or dysrhythmic eye blinks, lid openings and closures, eye blink rate, eye blink duration, and/or eye activity during sleep. In some embodiments, the characteristic association of rhythmic eye blinking with an epileptic event suggests that the neural substrate for blinking shares some common pathway with that involved in the generation of the corticoreticular epileptic discharge. As described herein, the term “rhythmic” refers to activity and/or patterns of waves of approximately constant frequency. As described herein, the term “dysrhythmic” refers to activity and/or patterns in which no stable rhythms are present.

In some embodiments, the one or more oculometric parameters include eyeball movements such as upward eyeball movements, downward eyeball movements, lateral eyeball movements, eye rolling, jerky eye movements, saccadic velocity, torsional velocity, saccadic direction, and/or torsional direction. There are different types of eye movements, including, but not limited to, saccadic, smooth pursuit, vergence, and vestibule ocular movements, associated with varying visual functions.

Saccades are fast movements of the eyes, which are employed to position the images of objects of interest onto the fovea of the eye. In some embodiments, the eyeball movements may be measured with a resolution of about 1 degree to about 3 degrees, inclusive, such as 1 degree to 1.5 degrees, 1 degree to 2 degrees, 1 degree to 2.5 degrees, or 1 degree to 3 degrees, inclusive.

In some embodiments, the one or more oculometric parameters include eye eccentricity. Eye eccentricity is a function of visible x width and y width of the pupil of an eye. In some aspects, eye eccentricity changes as the eyelid position, position of the sides of the eye, pupil area, and/or blink frequency change(s). As defined above, eye eccentricity is a parameter associated with every conic section. In exemplary embodiments, the measuring device measures the visible portion of the pupil as an approximated ellipse. In exemplary embodiments, the eccentricity of an ellipse is greater than zero but less than 1. An ellipse is a curve in a plane surrounding two focal points such that the sum of the distances to the two focal points is constant for every point on the curve. In some aspects, eye eccentricity combines multiple oculometric parameters.

In some embodiments, the one or more oculometric parameters include left and right eye movements. In some embodiments, the disclosed methods herein include measuring a change in one or more oculometric parameters of both the left eye and the right eye. In some embodiments, the disclosed methods herein further include cross-correlating oculometric data of a left eye and oculometric data of a right eye of the subject and determining the presence of an increase in the synchronization of eye movements between the left eye and the right eye of the subject relative to baseline.

In some embodiments, left and right eye movements are analyzed with a broad regression analysis to develop a correlation amplitude and time delay for the different variables.

In other embodiments, the disclosed methods herein provide a method of detecting and/or predicting an epileptic event in a subject including measuring left and right eye movements over time using a measuring device to obtain eye movement data from the subject; identifying the presence or absence of an increase in the correlation of left and right eye movements over time based on the measuring; and indicating that an epileptic seizure has been detected and/or predicted when the identifying indicates the presence of an increase in the correlation of left and right eye movements over time. In some embodiments, the disclosed methods herein further include cross-correlating eye movement data of a left eye and eye movement data of a right eye of the subject.

In certain aspects, the measuring device is configured to obtain eye movement data from the subject for about 30 minutes to about 60 minutes. In some embodiments, the measuring device is configured to obtain eye movement data from the subject, either continuously or intermittently, for a desired amount of minutes, hours, days, months, or years, such as about 5 minutes to 10 years, inclusive, including 5 minutes to 20 minutes, 5 minutes to 30 minutes, 5 minutes to 40 minutes, 5 minutes to 50 minutes, 5 minutes to 1 hour, 5 minutes to 10 hours, 5 minutes to 20 hours, 5 minutes to 1 day, 5 minutes to 10 days, 5 minutes to 20 days, 5 minutes to 1 month, 5 minutes to 5 months, 5 minutes to 10 months, 5 minutes to 1 year, 5 minutes to 2 years, 5 minutes to 5 years, or 5 minutes to 8 years, inclusive. In some aspects, the eye movement data from the subject is captured at about 30 frames per second (fps) or more. In other aspects, the eye movement data from the subject is captured at about 20 fps to about 400 fps, inclusive, such as 20 fps to about 60 fps, 20 fps to about 100 fps, 20 fps to about 150 fps, 20 fps to about 200 fps, 20 fps to about 300 fps, or 20 fps to about 400 fps.

In some embodiments, the in-sync behavior of the x-axis amplitude of the pupils of the left and right eyes changes during or after an epileptic event. In some embodiments, the in-sync behavior of the x-axis amplitude of the pupils of the left and right eyes are more in-phase relative to baseline during or after an epileptic event. In some embodiments, the in-sync behavior of the x-axis amplitude of the left and right eyes are less in-phase relative to baseline during or after an epileptic event. For example, the measurement of in-phase movements of the eyes may increase in generalized seizures, but be less in-phase in partial seizures or in Todd's paralysis.

Todd's paralysis represents focal weakness in a part of the body after a seizure. This weakness typically affects appendages and is localized to either the left or right side of the body and usually subsides completely within 48 hours. Todd's paralysis may also affect speech, eye position or gaze, or vision. In some embodiments, the eyes and face of a subject may show increased and/or decreased in-phase movement depending on the seizure type, or even a loss of in-phase movement, activity which correlates with a particular epileptic event.

### Facial Biometrics

As used herein, the term “facial biometrics,” refers to patterns of involvement of the facial muscles of a subject before, during, or after an epileptic event. Exemplary facial biometrics data include, but are not limited to, distance between the eyes; distance between the eyelids; width of the nose; center of the nose; depth of the eye sockets; shape of the cheekbones; length of the jawline; distance between the mouth edges; center of the mouth; and/or focal weakness.

In some embodiments, oculometric parameters and facial biometrics are measured before, during, and after an epileptic event to gather additional independent variables for statistical analysis. In some embodiments, the disclosed methods herein include measuring a change in one or more facial biometrics of the subject to provide facial biometrics data. In some embodiments, the disclosed methods herein further include performing a first order statistical analysis and/or second order statistical analysis of the facial biometrics data and determining the presence or absence of a change relative to baseline in the first order statistical analysis and/or second order statistical analysis s of the facial biometrics data.

In some embodiments, facial biometrics may add additional independent variables to produce a stronger alert system, e.g., a seizure detection alarm. For example, an epileptic event may manifest as the pulling of one side of the mouth or face, or change in expression or emotion, such as fear, or pain. In other embodiments, Todd's paralysis may occur after a partial seizure and may present with a change in oculometric and facial biometrics data, representing in the slowing of movements, decreased range of movements, and relative slackening of facial muscles.

In some embodiments, the occurrence of rapid forced blinking, usually involving both eyes and without involvement of other facial muscles, may be present at the onset of seizures. In some embodiments, the eyes are closed during an epileptic event resulting in no useable data to process. Facial biometrics may be measured using a suitable measuring device, e.g., a camera and/or movement sensor, to obtain facial biometrics data from the subject.

In some embodiments, the measuring device is configured to obtain facial biometrics data from the subject for about 30 minutes to about 60 minutes. In some embodiments, the measuring device is configured to obtain facial biometrics data from the subject, either continuously or intermittently, for a desired amount of minutes, hours, days, months, or years, such as about 5 minutes to 10 years, inclusive, including 5 minutes to 20 minutes, 5 minutes to 30 minutes, 5 minutes to 40 minutes, 5 minutes to 50 minutes, 5 minutes to 1 hour, 5 minutes to 10 hours, 5 minutes to 20 hours, 5 minutes to 1 day, 5 minutes to 10 days, 5 minutes to 20 days, 5 minutes to 1 month, 5 minutes to 5 months, 5 minutes to 10 months, 5 minutes to 1 year, 5 minutes to 2 years, 5 minutes to 5 years, or 5 minutes to 8 years, inclusive. In some aspects, the facial biometrics data from the subject is captured at about 30 frames per second (fps) or more. In other aspects, the facial biometrics data from the subject is captured at about 20 fps to about 400 fps, inclusive, such as 20 fps to about 60 fps, 20 fps to about 100 fps, 20 fps to about 150 fps, 20 fps to about 200 fps, 20 fps to about 300 fps, or 20 fps to about 400 fps.

### Prodromal Changes

As used herein, the term “prodromal changes” refers to events occurring prior to the onset of an epileptic event. Prodromal changes may occur one or more days before the epileptic event, one or more hours before the epileptic event, one or more minutes before the epileptic event, or one or more seconds before the epileptic event.

In some embodiments, oculometric and facial biometrics data are measured for prodromal changes that predict an epileptic event days and hours prior to onset. The possibility of forecasting the occurrence of epileptic events, through the recognition of events taking place prior to the onset of an epileptic event, would have a positive impact on treatment efficacy and quality of life. In some embodiments, longer recordings performed in conjunction with long-term video EEG monitoring may allow for longer term signals in oculometric and biometric data that correlate with the buildup to an epileptic event.

In some aspects, the disclosed methods herein include measuring prodromal changes of the oculometric data and/or facial biometrics data. In some aspects, the disclosed methods herein further include performing a first order statistical analysis and/or second order statistical analysis of the prodromal changes of the oculometric data and/or facial biometrics data, and determining the presence or absence of a change relative to baseline in the first order statistical analysis and/or second order statistical analysis of the prodromal changes of the oculometric data and/or facial biometrics data.

In some embodiments, subjects are characterized by and monitored for irritability and decreased tolerance, lasting several hours. In some embodiments, subjects experience and are monitored for fatigue. In some embodiments, subjects experience and are monitored for cognitive disturbances including, but not limited to, an increased latency in verbal and motor responses, clumsiness, short-term memory, and/or attention disturbances. In some embodiments, subjects experience and are monitored for anxiety or mood changes including, but not limited to, tension, uneasiness, apathy, and/or indifference. In some embodiments, depressive symptoms are more frequent than elation symptoms. Other less frequently reported prodromal changes include sleep disturbances, dysthermia, speech disturbances, voiding changes, gastrointestinal symptoms, and/or headache. Some subjects may frequently require and are monitored for the interruption of activities in order to sleep. Some may experience and are monitored for a subjective unusual and unexplained cold sensation. Others may have and are monitored for slurred speech or an increase in number and volume of urination.

In some embodiments, the frequency of prodromal changes is measured. In other embodiments, the duration of prodromal changes is measured. The duration may range from about 30 minutes to several hours. In some embodiments, the frequency of prodromal changes correlates with a type of epileptic event, such as an absence seizure. In some embodiments, the prevalence of prodromal symptoms is measured.

### Lower Order Statistical Analysis

In some embodiments, lower order statistical analysis includes a first order statistical analysis and/or a second order statistical analysis. Such lower order statistical analysis describes the position and width of a distribution and may be calculated linearly having a power of 1 and quadratically having a power of 2.

As summarized above, the subject methods of detecting and/or predicting an epileptic event in a subject, include measuring a change in one or more oculometric parameters of at least one eye and/or facial biometrics of the subject over time using a measuring device to obtain oculometric data and/or facial biometrics data from the subject; performing a first order statistical analysis and/or second order statistical analysis of the oculometric data and/or facial biometrics data; determining the presence or absence of a change relative to baseline in the first order statistical analysis and/or second order statistical analysis of the oculometric data and/or facial biometrics data; and indicating that an epileptic event has been detected and/or predicted when the determining indicates the presence or absence of a change in the first order statistical analysis and/or second order statistical analysis relative to baseline. In certain aspects, the first order statistical analysis includes multiple regression analysis. Other examples of first order statistical analysis include mean calculations. In some embodiments, the second order statistical analysis includes variance calculations.

In certain aspects, performing the first order statistical analysis and/or second order statistical analysis of the oculometric data and/or facial biometrics data includes an analysis of oculometric and/or facial biometrics data collected over about a 1-second to a 15-second window, inclusive, such as a 1-second to a 3-second window, a 1-second to a 4-second window, a 1-second to a 5-second window, a 1-second to a 6-second window, a 1-second to a 7-second window, an 1-second to an 8-second window, a 1-second to a 9-second window, or a 1-second to a 10-second window. In some aspects, performing the first order statistical analysis and/or second order statistical analysis of the oculometric data and/or facial biometrics data includes an analysis of oculometric and/or facial biometrics data collected over a ten-second running window. In other aspects, performing the first order statistical analysis and/or second order statistical analysis of the oculometric data and/or facial biometrics data includes an analysis of oculometric and/or facial biometrics data collected over a five-second running window.

In some embodiments, performing the first order statistical analysis of the oculometric and/or facial biometrics data includes performing multiple regression analysis of the oculometric data and/or facial biometrics data. As described herein, the term “multiple regression analysis” refers to the relationship between one continuous dependent variable and two or more independent variables. The variable whose value is to be predicted is known as the dependent variable and the ones whos known values are used for prediction are known as independent variables. For example, the correlation between eye eccentricity and/or eye movements and an epileptic event may be ascertained using a multiple regression analysis.

In some embodiments, the subject methods include determining the presence or absence of a change relative to baseline in the first order statistical analysis and/or second order statistical analysis of the oculometric data and/or facial biometrics data. As defined above, the baseline may be patient/subject-specific. In some embodiments, the baseline for may be verified as occurring in the absence of an epileptic event, e.g., vie EEG measurement. In certain aspects, determining the presence or absence of a change in the first order statistical analysis and/or second order statistical analysis of the oculometric data and/or facial biometrics data includes determining the presence or absence of an increased correlation of one or more oculometric parameters and/or facial biometrics with the epileptic event. Correlation is any of a broad class of statistical relationships involving dependence. In other aspects, determining the presence or absence of an increased correlation of one or more oculometric parameters and/or facial biometrics with the epileptic event includes determining the presence or absence of an increased correlation of eye eccentricity with the epileptic event. For example, a broad regression analysis of the recorded oculometrics and facial biometrics may determine that the distribution of eye eccentricity correlates with epileptic event activity.

### Higher Order Statistical Analysis

In some embodiments, the subject methods further include performing a higher order statistical analysis of the oculometric data and/or facial biometrics data. A higher order statistical analysis refers to functions that use a third or higher power of a sample, as opposed to a first order statistical analysis or a second order statistical analysis, which uses constant, linear, and quadratic terms. Examples of a higher order statistical analysis include kurtosis and skewness. Higher order statistical analysis may be performed using bispectral analysis, a generalized linear and/or nonlinear regression analysis.

In some embodiments, the disclosed methods include determining the presence or absence of a change relative to baseline in the higher order statistical analysis of the oculometric data and/or facial biometrics data. In such cases, a higher order statistical analysis may measure the deviation of a distribution from a normal distribution. For example, the kurtosis of a normal distribution is 3. In certain aspects, determining the presence or absence of a change relative to baseline in the higher order statistical analysis of the oculometric data and/or facial biometrics data includes determining the presence of a change from frequency independence to inter-frequency dependence of the oculometric data. Determining the presence or absence of a change in the higher order statistical analysis of the oculometric data and/or facial biometrics data may additionally or alternatively include determining the presence of a change in synchronization such as frequency synchronization, including, but not limited to, dependent frequencies and/or uncoupled frequencies, before an epileptic event, during the epileptic event, or after an epileptic event. Determining the presence or absence of a change in the higher order statistical analysis of the oculometric data and/or facial biometrics data may additionally or alternatively include determining the presence of positive excess kurtosis of the oculometric data. In some embodiments, determining the presence of positive excess kurtosis of the oculometric data and/or facial biometrics data includes determining the presence of positive excess kurtosis of eye eccentricity. Excess kurtosis is a statistical term describing that a probability has a kurtosis coefficient that is larger than the coefficient associated with a normal distribution, which is 3 as set forth above. In certain aspects, the positive excess kurtosis is about 5 to about 20, inclusive, such as 5 to about 10, 5 to about 15, or 5 to about 20. In other aspects, the positive excess kurtosis is 15 or more.

In some embodiments, kurtosis of the oculometric data and/or facial biometrics data is measured in about a 1-second to about a 15-second window, inclusive, such as a 1-second to a 3-second window, a 1-second to a 4-second window, a 1-second to a 5-second window, a 1-second to a 6-second window, a 1-second to a 7-second window, an 1-second to an 8-second window, a 1-second to a 9-second window, or a 1-second to a 10-second window. In some embodiments, kurtosis measurements are performed in a 5-second window. In some embodiments, kurtosis of the oculometric data and/or facial biometrics data is measured in about a 2-second to a 8-second window, e.g., a 4-second to a 6-second window.

### Cross-Correlation

As described herein, the terms “cross-correlation” and “cross-correlating” are used interchangeably to refer to a technique used to measure the relationship between two or more variables. Cross-correlation is a measure of similarity of two series as a function of the displacement of one relative to the other. The degree to which two series are correlated may be measured. A time series is a series of data points indexed in time order. Correlation may be both a measure of similarity between portions of two time series and the lag time between the correlated portions. Cross-correlation may be a function of amplitude of correlation versus the lag time between the correlated portions. For example, if there is a similar structure potentially representing a similar event in the two time series, but the similar portions are separated, or delayed by, 5 seconds, for instance, there will be a higher magnitude, which varies between 0 and 1, of correlation appearing at the lag time of 5 seconds.

In some embodiments, the disclosed methods include cross-correlating oculometric data and/or eye movement data of a left eye and oculometric data and/or eye movement data of a right eye of the subject. In certain aspects, the cross-correlation of the oculometric data and/or eye movement data of a left eye and oculometric data and/or eye movement data of a right eye of the subject yields about a 10% to about a 50% change in cross-correlation, inclusive, such as a 10% to about a 20% change, a 10% to about a 30% change, a 10% to about a 40% change, or a 10% to about a 50% change.

In some embodiments, the disclosed methods include cross-correlating the first order analysis and/or second order statistical analysis of the oculometric data and/or facial biometrics data. In certain aspects, the cross-correlation of the first order analysis of the oculometric data and/or facial biometrics data yields about a 10% to about a 50% change in cross-correlation, inclusive, such as a 10% to about a 20% change, a 10% to about a 30% change, a 10% to about a 40% change, or a 10% to about a 50% change.

In some embodiments, the disclosed methods include cross-correlating the higher order analysis of the oculometric data and/or facial biometrics data. In certain aspects, the cross-correlation of the higher order analysis of the oculometric data and/or facial biometrics data yields about a 10% to about a 50% change in cross-correlation, inclusive, such as a 10% to about a 20% change, a 10% to about a 30% change, a 10% to about a 40% change, or a 10% to about a 50% change.

### Synchronization

As described herein, the term “synchronization” refers to the coordination of two or more variables in time. Synchronization of data includes frequency synchronization of the data. For example, during an epileptic event, an increase in the frequency of eye movement may synchronize with the frequency of mouth movement. Changes in synchronization may occur before, during, or after the occurrence of an epileptic event. Synchronization may be performed using standard techniques known in the art, including, but not limited to, Fujisaka and Yamada (1983), Afraimovich et al. (1986), and Rosenblum et al. (1996), the disclosures of which are incorporated herein by reference.

In some embodiments, determining the presence or absence of the change relative to baseline in the first order statistical analysis and/or second order statistical analysis of the oculometric data includes determining the presence of an increase in the synchronization of eye movements between the left eye and the right eye of the subject relative to baseline.

In some embodiments, determining the presence or absence of a change relative to baseline in the higher order statistical analysis of the oculometric data and/or facial biometrics data includes determining a change in synchronization of the oculometric data and/or facial biometrics data. In certain aspects, determining synchronization of the oculometric data and/or facial biometrics data includes determining frequency synchronization of the oculometric data and/or facial biometrics data. Synchronization of the oculometric data and/or facial biometrics data may include parameters that are different. In some embodiments, a frequency distribution may illustrate grouping of data divided into mutually exclusive classes and the number of occurrences in each class.

In certain aspects, determining frequency synchronization includes determining synchronization of dependent and/or uncoupled frequencies of the oculometric data and/or facial biometrics data. A frequency of an event is the number of times the event occurs over time. Certain frequencies may be dependent and/or uncoupled in relationship to each other. For example, the increased frequency of eye lid movement depends on the increased frequency of eye ball movement during an epileptic event, but is uncoupled, or otherwise, not affected, by the increased frequency of mouth movement.

### Machine Learning

In practicing the subject methods, determining the presence or absence of a change in a lower order statistical analysis and/or a higher order statistical analysis may utilize machine learning. Machine learning techniques and computational methods may be used for predicting epileptic seizures from the data obtained. In some embodiments, the disclosed methods herein include two types of data. For example, oculometrics and facial biometrics measurements and subsequent statistical analysis produce numerical data. The clinical read of the EEG for epileptic events and type of epileptic events, or the “outcome”, e.g., seizure onset, produce categorical data. The machine learning process may involve relating the numerical data to the outcomes, which applies categorical training to detect and/or predict an epileptic event.

In certain aspects, machine learning models are used to predict epileptic seizures. The machine learning models may include EEG signal acquisition, signal preprocessing, features extraction from the signals, and classification between different seizure states. In some embodiments, the disclosed methods herein include measuring at least one EEG signal of the subject. In such cases, the disclosed methods herein may include confirming the presence or absence of a change relative to baseline in the lower order statistical analysis and/or a higher order statistical analysis of the oculometric data using the at least one electroencephalogram signal. In other embodiments, the epileptic event in the subject is detected and/or predicted in the absence of measuring an electroencephalogram signal of the subject. In such cases, the data in a time series may be analyzed by a lower order statistical analysis and/or a higher order statistical analysis including, but not limited to, mean, standard deviation, kurtosis, and dominant frequencies from spectral analysis. For example, a sequence of learning procedures listed by increasing processing complexity may be numerical data obtained from a measuring device analyzed using a lower order statistical analysis and/or a higher order statistical analysis, categorical outcomes produced by a clinical read of the EEG, and lastly, numerical data including or excluding EEG as related to categorical data. In certain aspects, the disclosed methods herein utilize machine learning algorithms embedded in-line with the disclosed methods to enhance clinical practices in identifying subjects as having an epileptic event and/or as at risk of an epileptic event.

In some embodiments, machine learning algorithms involve thresholding as determined by a statistical reliability of outcomes. In some embodiments, a portion of the data obtained, e.g. 70 percent, may be used for training and the remaining data for testing and determining statistical analysis of outcomes. In such cases, the data breakdown is analogous to a standard 2×2 decision theory representation of true/false positives and true/false negatives. For example, a receiver operating characteristic curve (ROC curve) may be created to illustrate the true positive rate against the false positive rate at various threshold settings. The true-positive rate is also known as sensitivity, recall or probability of detection in machine learning.

Other exemplary tools to determine thresholding to maximize sensitivity and specificity include MATLAB's Statistics and Machine Learning Toolbox™, Neural Network Toolbox™, Image Processing Toolbox™, the Image Acquisition Toolbox™, and the Mapping Toolbox™. The thresholding to maximize sensitivity and specificity is dependent on the epileptic event type. For example, seizure types with higher morbidity may set a higher sensitivity and lower specificity. Similarly, seizures with lower morbidity such as in absence seizures, may utilize a higher specificity and lower sensitivity setting.

### Alerts

The present disclosure provides an epileptic event alert mechanism/alarm. In some embodiments, the subject methods include indicating that an epileptic event has been detected and/or predicted when the determining indicates the presence or absence of a change in the lower order statistical analysis and/or a higher order statistical analysis relative to baseline. In certain aspects, indicating that the epileptic event has been detected and/or predicted includes providing an alert to the subject or a caregiver of the subject. An alert may be provided in any suitable format, e.g., as an audio alert, a visual alert, and/or a tactile alert. Such alerts may be provided by any suitable output device, e.g., a handheld device, such as a smartphone; a wearable device such as an Apple® Watch or equivalent, etc. In other embodiments, the indicating further includes providing a responsive neurostimulation to the subject, wherein the responsive neurostimulation is sufficient to reduce the effect of the epileptic event, when the epileptic event is detected and/or predicted. Specifically, an electric current may be transmitted through the neck of the diagnosed subject to a vagus nerve in the diagnosed subject, wherein the electric current is sufficient to terminate the epileptic event, when the epileptic event is detected and/or predicted. In other aspects, an effective amount of an anti-epileptic drug may be administered to the subject, when the epileptic event is detected and/or predicted.

In some embodiments, the epileptic event alarm includes an algorithm composed of the multiple regression analysis of the oculometric and/or facial biometrics data, such as eye eccentricity and in-sync eye movements, that timelock with epileptic events on the EEG. The epileptic event alarm may be validated by predicting the EEG data. In some embodiments, the epileptic event alarm may be commercially developed to use oculometric and facial biometrics data analyzed from a camera and in real-time produce an alarm that can be sent to a smartphone alert system to the subject's family, to medical personnel, or to emergency services via a communication unit. In some embodiments, the alerted persons administer rescue medications. In other embodiments, the alarm may be used in closed loop systems including, but not limited to, vagus nerve stimulation (VNS) or responsive neurostimulation (RNS) to deliver a signal to reduce the effect of the epileptic event or terminate the epileptic event, when the epileptic event is detected and/or predicted.

In certain embodiments, a treatment for epilepsy is via open loop VNS, a reversible procedure which introduces an electronic device that employs a pulse generator and an electrode to alter neural activity. The vagus nerve is a major nerve pathway that emanates from the brainstem and passes through the neck to control visceral function in the thorax and abdomen. VNS uses open looped, intermittent stimulation of the left vagus nerve in the neck in an attempt to reduce the frequency and intensity of seizures.

In some embodiments, the disclosed methods herein may include transmitting a local alert signal which the subject may switch off in case of a false alarm, before the alert is transmitted to a remote location, such as to the subject's family, to medical personnel, or to emergency services. A predefined time is allowed to pass before the remote alert is sent, to allow the subject sufficient time to deactivate a false alarm.

In certain embodiments, a communication unit includes a communication circuit selected from a Bluetooth circuit, WiFi circuit, a ZigBee, and/or a GPRS circuit. In some embodiments, the disclosed methods herein may further include instructing a treatment unit to administer an epileptic treatment in response to an alert signal. The treatment unit may apply a treatment automatically in response to either a local or remote alert signal, or may be adapted to be triggered by a treatment signal initiated remotely and received through the communication unit. In some embodiments, the disclosed methods may further include detecting sounds originating by a subject and from the vicinity of the subject, and the communication unit is adapted to transmit the sounds to the treatment unit as detected by a microphone.

### Pharmaceutical Treatments

In certain aspects, the disclosed methods herein include administering an effective amount of an anti-epileptic drug to the subject, when the epileptic event is detected and/or predicted. For example, a method of identifying and treating epilepsy in a subject includes measuring a change in one or more oculometric parameters of at least one eye and/or facial biometrics of the subject over time using a measuring device to obtain oculometric data and/or facial biometrics data from the subject; performing a lower order statistical analysis and/or a higher order statistical analysis of the oculometric data and/or facial biometrics data; determining the presence or absence of a change relative to baseline in the lower order statistical analysis and/or a higher order statistical analysis of the oculometric data and/or facial biometrics data; identifying the subject as having an epileptic event and/or as at risk of an epileptic event when the determining indicates the presence or absence of a change in the lower order statistical analysis and/or a higher order statistical analysis of the oculometric data and/or facial biometrics data relative to baseline; and administering an effective amount of an anti-epileptic drug to the subject identified as having an epileptic event and/or as at risk of an epileptic event.

Suitable anti-epileptic drugs which may be used in the context of the disclosed methods and systems include anticonvulsant drugs. For generalized tonic-clonic seizures, rescue medications such as lorazepam, diazepam, midazolam, clonazepam or standard prophylactic anticonvulsants such as lamotrigine, leviteracetam, lacosamide or valproate may be administered. For partial seizures, medications, including, but not limited to, those used for treating generalized tonic-clonic seizures may be administered. Treatment may begin with carbamazepine, phenytoin, or valproate. If seizures persist despite high doses of these drugs, lamotrigine, or topiramate may be added. For absence seizures, ethosuximide orally may be administered. Valproate and clonazepam orally may also be effective. Acetazolamide may be used for refractory cases. Atonic seizures, myoclonic seizures, and infantile spasms are difficult to treat. Valproate may be utilized, followed, if unsuccessful, by clonazepam. Ethosuximide is sometimes effective, as is acetazolamide (in dosages as for absence seizures). For infantile spasms, corticosteroids for 8 to 10 weeks are often effective.

In some embodiments, an effective amount of an anti-epileptic drug to the subject may be administered. Exemplary anti-epileptic drugs administered include intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.

Therapeutic agents can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant for example.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

Toxicity and therapeutic efficacy of the active ingredient can be determined according to standard pharmaceutical procedures in cell cultures and/or experimental animals, including, for example, determining the LD50 (the dose lethal to 50% of the population, or for the methods of the invention, may alternatively by the kindling dose) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The anti-epileptic drugs described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the anti-epileptic drugs are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The anti-epileptic drugs may be administered using any medically appropriate procedure, e.g. intravascular (intravenous, intraarterial, intracapillary) administration, injection into the cerebrospinal fluid, intracavity or direct injection in the brain. Intrathecal administration may be carried out through the use of an Ommaya reservoir, in accordance with known techniques. (F. Balis et al., Am J. Pediatr. Hematol. Oncol. 11, 74, 76 (1989).

The effective amount of an anti-epileptic drug to be given to a particular subject will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD50 animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent.

### Subjects Suitable for Treatment

A variety of subjects (wherein the term “subject” is used interchangeably herein with the terms “host” and “patient”) are treatable according to the methods of the present disclosure. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), non-human primates, and primates (e.g., humans, chimpanzees, and monkeys). In some cases, a suitable subject for treatment methods of the present disclosure is a human.

Subjects suitable for treatment with a subject method include individuals who have been identified as having an epileptic event and/or as at risk of an epileptic event. Subjects having epilepsy experience sudden recurrent episodes of sensory disturbance, loss of consciousness, and/or convulsions. Treatment of subjects as having an epileptic event and/or as at risk of an epileptic event is of particular interest.

In some cases, subjects suitable for treatment using methods of the present disclosure include individuals diagnosed with epilepsy, e.g., generalized epilepsy or focal epilepsy. In some cases, subjects suitable for treatment using methods of the present disclosure include individuals diagnosed with drug resistant epilepsy, and are suitable for treatment using methods of the present disclosure.

### Systems

The disclosed systems of detecting and/or predicting an epileptic event in a subject include a measuring device configured to measure a change in one or more oculometric parameters of at least one eye and/or facial biometrics of the subject over time; a processor unit; a non-transitory computer-readable storage medium comprising instructions, which when executed by the processor unit, cause the processor unit to perform a lower order statistical analysis and/or a higher order statistical analysis of the oculometric data and/or facial biometrics data and determine the presence or absence of a change relative to baseline in the lower order statistical analysis and/or a higher order statistical analysis of the oculometric data and/or facial biometrics data; and an output device configured to indicate that an epileptic event has been detected and/or predicted when a change in the lower order statistical analysis and/or a higher order statistical analysis is determined to be present.

In certain aspects, the one or more oculometric parameters includes, but is not limited to, eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep. In other aspects, the measuring device measures a change in two or more of the oculometric parameters. In some embodiments, the one or more oculometric parameters includes eye eccentricity. In some embodiments, eye eccentricity changes as the eyelid position, position of the sides of the eye, pupil area, and/or blink frequency change(s).

In other aspects, the one or more facial biometrics includes, but is not limited to, distance between the eyes; distance between the eyelids; width of the nose; center of the nose; depth of the eye sockets; shape of the cheekbones; length of the jawline; distance between the mouth edges; center of the mouth; and/or focal weakness.

### Measuring Devices

The disclosed systems include and the disclosed methods utilize one or more measuring devices. In some embodiments, the measuring device is configured to obtain oculometric data, facial biometrics data, and/or eye movement data from the subject, either continuously or intermittently, for a desired amount of minutes, hours, days, months, or years, such as about 5 minutes to 10 years, inclusive, including 5 minutes to 20 minutes, 5 minutes to 30 minutes, 5 minutes to 40 minutes, 5 minutes to 50 minutes, 5 minutes to 1 hour, 5 minutes to 10 hours, 5 minutes to 20 hours, 5 minutes to 1 day, 5 minutes to 10 days, 5 minutes to 20 days, 5 minutes to 1 month, 5 minutes to 5 months, 5 minutes to 10 months, 5 minutes to 1 year, 5 minutes to 2 years, 5 minutes to 5 years, or 5 minutes to 8 years, inclusive. In some aspects, the oculometric data, facial biometrics data, and/or eye movement data from the subject is captured at about 30 frames per second (fps) or more. In other aspects, the oculometric data, facial biometrics data, and/or eye movement data from the subject is captured at about 20 fps to about 400 fps, inclusive, such as 20 fps to about 60 fps, 20 fps to about 100 fps, 20 fps to about 150 fps, 20 fps to about 200 fps, 20 fps to about 300 fps, or 20 fps to about 400 fps. In other embodiments, the measuring device is configured to measure prodromal changes of the oculometric data, facial biometrics data, and/or eye movement data. Such prodromal changes may occur one or more days before, one or more hours before, r one or more seconds before an epileptic event.

In some embodiments, the measuring device is an eye tracking device. The eye tracking device may include one or more cameras, or may further include a video recorder and/or a sensor. In some embodiments, the eye tracking device is a wearable device configured to be worn on the head of the subject. In certain aspects, the one or more cameras of the wearable device are located at a distance of one or more centimeters from the eyes of the subject. In some embodiments, the wearable device is a conventional video camera, an Eye-Com Biosensor™ such as the Model EC-7T system, a GoPro® camera, or Pupil Labs Pupi™.

In some embodiments, the Eye-Com Biosensor™ or an equivalent device may track real-time ictal and postictal manifestations of seizures. The Model EC-7T system uses frame-mounted micro-cameras located in an eye-frame at a distance of 1 to 2 centimeters from the eyes of the subject. In certain aspects, the system may record one or more oculometric parameters of at least one eye, facial biometrics, and/or eye movement data at very close distance continuously. In other aspects, the system may record changes in oculometrics, facial biometrics, and/or eye movements related to seizures and paroxysmal events, including autonomic changes before during and after seizures. In some embodiments, the system includes a portable pair of glasses that can be adapted to fit neonates and adults in the home as well as the hospital setting. In some embodiments, the system may interact with other devices such as a computer interface that can present the subject with commands to follow and simultaneously determine whether there is impairment in consciousness.

In some embodiments, GoPro® cameras are small, rugged, waterproof, and may come with an array of mounting geometries. GoPro® cameras or equivalent devices may film and record many activities including tracking the eyes of a subject. Many GoPro® devices include a liquid-crystal display (LCD) screen that may attach to the back of the camera. Commercial GoPro® cameras include, but are not limited to, the HD HERO™ series, the HERO™ series, and the HERO+™ series.

In some embodiments, the Pupil Labs Pupil™ or an equivalent device may use mobile eye tracking and gaze-based interaction. The system includes a headset with high-resolution cameras, an open source software framework for mobile eye tracking, and a graphical user interface to playback and visualize video and gaze data. Features include high-resolution scene and eye cameras for monocular and binocular gaze estimation. The mobile eye tracking headset may have one scene camera and one infrared spectrum eye camera for dark pupil detection. Both cameras may connect to a computer interface. The camera video streams may be read using Pupil Labs™ software for real-time pupil detection, gaze mapping, recording, and other functions. Other exemplary add-on features include virtual reality and augmented reality platforms.

In other embodiments, the eye tracking device is a contact lens, for e.g., as described in US 20170049395, the disclosure of which is incorporated herein by reference. In certain aspects, the eye tracking device includes at least one sensor and is configured to couple with a power source and a processor configured to process data generated by the at least one sensor. In such cases, oculometrics using a contact lens measuring pupil diameter and location may pick up signals associated with changes in eye activity during sleep, correlating with central apneas or cardiac arrhythmias which may be related to SUDEP.

In certain aspects, the measuring device may be designed based on micro-electromechanical systems (MEMS) technology developed on a film of contact lens material forming the lens, including, but not limited to polydimethylsiloxane (PDMS). The operating frequency may utilize near field communication (NFC), including an NFC frequency of 13.56 Hz, for example. In some embodiments, the measuring device is further coupled to a miniaturized coil and a power coil.

Other examples of measuring devices may include one or more cameras mounted on the clothing of a subject, Google Glass™, a wearable device with one or more cameras mounted inside for sleeping, and/or one or more video recorders located close to the eyes and face of the subject. Such devices monitor in real-time the eyes and face of a subject. In some embodiments, oculometric data, facial biometrics data, and/or eye movement data are monitored and recorded in synchrony with EEG signals. In certain aspects, the disclosed systems may further include an input device configured to measure at least one EEG signal on the subject. In other aspects, the epileptic event in the subject is detected and/or predicted in the absence of measuring an electroencephalogram signal of the subject.

### Processor Units

As summarized above, the subject systems include a processor unit and a non-transitory computer-readable storage medium comprising instructions, which when executed by the processor unit, cause the processor unit to perform a first order statistical analysis and/or second order statistical analysis of the oculometric data and/or facial biometrics data, and determine the presence or absence of a change relative to baseline in the first order statistical analysis of the oculometric data and/or facial biometrics data. In some embodiments, the first order statistical analysis performed includes multiple regression analysis and/or mean calculations of the oculometric data and/or facial biometrics data. In other embodiments, the second order statistical analysis performed includes determining the variance calculations of the oculometric data and/or facial biometrics data.

In certain aspects, the non-transitory computer-readable storage medium includes instructions, which when executed by the processor unit, cause the processor unit to perform the first order statistical analysis and/or second order statistical analysis of the oculometric data and/or facial biometrics data in a fifteen-second running window. In certain aspects, the non-transitory computer-readable storage medium includes instructions, which when executed by the processor unit, cause the processor unit to perform the first order statistical analysis and/or second order statistical analysis of the oculometric data and/or facial biometrics data in a ten-second running window. In other aspects, the non-transitory computer-readable storage medium includes instructions, which when executed by the processor unit, cause the processor unit to perform the first order statistical analysis and/or second order statistical analysis of the oculometric data in a five-second running window.

In some embodiments, the non-transitory computer-readable storage medium including instructions, which when executed by the processor unit, cause the processor unit to perform the first order statistical analysis and/or second order statistical analysis of the oculometric data includes performing multiple regression analysis, mean calculations, and/or variance calculations of the oculometric data. In some embodiments, determining the presence or absence of a change in the first order statistical analysis and/or second order statistical analysis of the oculometric data includes determining the presence or absence of an increased correlation of one or more oculometric parameters with the epileptic event. Specifically, determining the presence or absence of an increased correlation of one or more oculometric parameters with the epileptic event includes determining the presence or absence of an increased correlation of eye eccentricity with the epileptic event.

In certain aspects, the system includes (or the methods utilize) a measuring device further configured to measure a change in one or more facial biometrics of the subject to provide facial biometrics data. In such cases, the non-transitory computer readable storage medium further includes instructions, which when executed by the processor unit, cause the processor unit to perform a first order statistical analysis of the facial biometrics data. In some embodiments, the non-transitory computer readable storage medium further includes instructions, which when executed by the processor unit, cause the processor unit to determine the presence or absence of a change relative to baseline in the first order statistical analysis of the facial biometrics data. As summarized above, the one or more facial biometrics includes distance between the eyes; distance between the eyelids; width of the nose; center of the nose; depth of the eye sockets; shape of the cheekbones; length of the jawline; distance between the mouth edges; center of the mouth; and/or focal weakness.

In other embodiments, the non-transitory computer readable storage medium further includes instructions, which when executed by the processor unit, cause the processor unit to perform a first order statistical analysis and/or second order statistical analysis of the prodromal changes of the oculometric data and/or facial biometrics data. In some embodiments, the non-transitory computer readable storage medium further includes instructions, which when executed by the processor unit, cause the processor unit to determine the presence or absence of a change relative to baseline in the first order statistical analysis and/or second order statistical analysis of the prodromal changes of the oculometric data and/or facial biometrics data.

In some embodiments, the non-transitory computer-readable storage medium includes instructions, which when executed by the processor unit, cause the processor unit to cross-correlate oculometric data and/or eye movement data of a left eye and oculometric data and/or eye movement data of a right eye of the subject. In some embodiments, determining the presence or absence of the change relative to baseline in the first order statistical analysis and/or second order statistical analysis of the oculometric data includes determining the presence of an increase in the synchronization of eye movements between the left eye and the right eye of the subject relative to baseline.

In some embodiments, the non-transitory computer-readable storage medium includes instructions, which when executed by the processor unit, cause the processor unit to cross-correlate the first order statistical analysis and/or the second order stiatistical analysis of the oculometric data and/or facial biometrics data. In certain aspects, the higher order statistical analysis of the oculometric data and/or facial biometrics data includes cross-correlating the first statistical analysis and/or second statistical analysis of one or more oculometric parameters and/or facial biometrics that are different.

In some embodiments, the non-transitory computer readable storage medium further includes instructions, which when executed by the processor unit, cause the processor unit to perform a higher order statistical analysis of the oculometric data and/or facial biometrics data. As defined above, the higher order statistical analysis of the oculometric data and/or facial biometrics data may include kurtosis. In certain aspects, the non-transitory computer readable storage medium further includes instructions, which when executed by the processor unit, cause the processor unit to determine the presence or absence of a change relative to baseline in the higher order statistical analysis of the oculometric data and/or facial biometrics data. In some such embodiments, determining the presence or absence of a change relative to baseline in the higher order statistical analysis of the oculometric data and/or facial biometrics data includes determining the presence of a change from frequency independence to inter-frequency dependence of the oculometric data. In some such embodiments, determining the presence or absence of a change relative to baseline in the higher order statistical analysis of the oculometric data and/or facial biometrics data includes determining change in synchronization of the oculometric data and/or facial biometrics data. In certain aspects, determining synchronization of the oculometric data and/or facial biometrics data includes determining frequency synchronization of the oculometric data and/or facial biometrics data, including, but not limited to, determining synchronization of dependent and/or uncoupled frequencies of the oculometric data and/or facial biometrics data. In other embodiments, determining the presence or absence of a change in the higher order statistical analysis of the oculometric data and/or facial biometrics data includes determining the presence of positive excess kurtosis of the oculometric data and/or facial biometrics data. Specifically, determining the presence of positive excess kurtosis of the oculometric data may include determining the presence of positive excess kurtosis of eye eccentricity. In certain aspects, the positive excess kurtosis of the oculometric data and/or facial biometrics data is about 5 to about 20, inclusive, such as 5 to about 10, 5 to about 15, or 5 to about 20. In other aspects, the positive excess kurtosis is 15 or more. In some embodiments, the processor unit includes a memory field for containing a computer interface.

In some embodiments, the non-transitory computer-readable storage medium includes instructions, which when executed by the processor unit, cause the processor unit to cross-correlate the higher order stiatistical analysis of the oculometric data and/or facial biometrics data. In certain aspects, the higher order statistical analysis of the oculometric data and/or facial biometrics data includes cross-correlating the higher statistical analysis of one or more oculometric parameters and/or facial biometrics that are different.

In some embodiments, the non-transitory computer-readable storage medium including instructions, which when executed by the processor unit, cause the processor unit to confirm the presence or absence of a change relative to baseline in the first order statistical analysis, second order statistical analysis, and/or a higher order statistical analysis of the oculometric data and/or facial biometrics data using the at least one electroencephalogram signal. In other embodiments, the epileptic event in the subject is detected and/or predicted in the absence of measuring an electroencephalogram signal of the subject.

The disclosed systems may also be aided by machine learning. Such systems are capable of analyzing whether the data gathered is similar to that occurring in an epileptic event and dissimilar to that seen in a plurality of everyday activities which an individual may undertake. The system utilizes computerized processing to evaluate the data characteristics. In some embodiments, an alert signal may be transmitted to the individual's family, to medical personnel or to emergency services via an output device.

### Output Devices

Embodiments of the present invention may include devices for performing the operations herein including an output device configured to indicate that an epileptic event has been detected and/or predicted when a change in the first order statistical analysis, second order statistical analysis, and/or a higher order statistical analysis is determined to be present. In certain aspects, the output device configured to indicate that the epileptic event has been detected and/or predicted includes providing an alert to the subject or a caregiver of the subject. An alert may be provided in any suitable format, e.g., as an audio alert, a visual alert, and/or a tactile alert. Such alerts may be provided by any suitable output device, e.g., a handheld device, such as a smartphone; a wearable device such as an Apple® Watch or equivalent, etc. In other aspects, the disclosed systems include a neurostimulation device configured to provide a responsive neurostimulation to the subject, wherein the responsive neurostimulation is sufficient to reduce the effect of the epileptic event, when the epileptic event is detected and/or predicted. In such embodiments, a neurostimulation device is configured to provide an electric current through the neck of the diagnosed subject to a vagus nerve in the diagnosed subject, wherein the electric current is sufficient to terminate the epileptic event, when the epileptic event is detected and/or predicted.

In some embodiments, a drug administration device is configured to administer an effective amount of an anti-epileptic drug to the subject, when the epileptic event is detected and/or predicted. As summarized above, the anti-epileptic drug includes one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.

The output device may be specially constructed for the desired purposes, or it may include a general purpose computer selectively activated or reconfigured by a computer program stored in the computer. The output device may include a memory field for containing a computer interface. Such a computer program may be stored in a non-transitory computer readable storage medium, such as, but is not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system.

The methods and systems presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-282 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of detecting and/or predicting an epileptic event in a subject, the method comprising:
   a) measuring a change in one or more oculometric parameters of at least one eye of the subject overtime using a measuring device to obtain oculometric data from the subject;
   b) performing a first order statistical analysis of the oculometric data;
   c) determining the presence or absence of a change relative to baseline in the first order statistical analysis of the oculometric data; and
   d) indicating that an epileptic event has been detected and/or predicted when the determining indicates the presence or absence of a change in the first order statistical analysis relative to baseline.
2. The method of aspect 1, wherein the one or more oculometric parameters comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.
3. The method of aspect 1 or 2, wherein the measuring comprises measuring a change in two or more of the oculometric parameters.
4. The method of any one of aspects 1-3, wherein eye eccentricity is a function of visible x-width and y-width of the pupil of an eye.
5. The method of aspect 4, wherein eye eccentricity changes as the eyelid position, position of the sides of the eye, pupil area, and/or blink frequency change(s).
6. The method of any one of aspects 1-5, wherein the first order statistical analysis of the oculometric data comprises multiple regression analysis and/or mean calculations of the oculometric data.
7. The method of any one of aspects 1-6, wherein the measuring device is configured to obtain oculometric data from the subject for about thirty minutes.
8. The method of any one of aspects 1-6, wherein the measuring device is configured to obtain oculometric data from the subject for about fifteen minutes.
9. The method of aspect 7 or 8, wherein the performing the first order statistical analysis of the oculometric data occurs in a ten-second running window.
10. The method of aspect 7 or 8, wherein the performing the first order statistical analysis of the oculometric data occurs in a five-second running window.
11. The method of any one of aspects 1-10, wherein the measuring device is an eye tracking device.
12. The method of aspect 11, wherein the eye tracking device comprises one or more cameras.
13. The method of aspect 12, wherein the eye tracking device further comprises a video recorder and/or a sensor.
14. The method of aspect 13, wherein the eye tracking device is a wearable device configured to be worn on the head of the subject.
15. The method of aspect 14, wherein the one or more cameras of the wearable device is located at a distance of one or more centimeters from the eyes of the subject.
16. The method of any one of aspects 1-13, wherein the eye tracking device is a contact lens.
17. The method of any one of aspects 1-16, wherein the performing the first order statistical analysis of the oculometric data comprises performing multiple regression analysis of the oculometric data.
18. The method of aspect 17, wherein the determining the presence or absence of a change in the first order statistical analysis of the oculometric data comprises determining the presence or absence of an increased correlation of one or more oculometric parameters with the epileptic event.
19. The method of aspect 18, wherein the determining the presence or absence of an increased correlation of one or more oculometric parameters with the epileptic event comprises determining the presence or absence of an increased correlation of eye eccentricity with the epileptic event.
20. The method of any one of aspects 1-19, wherein the oculometric data from the subject is captured at about 30 frames per second or more.
21. The method of any one of aspects 1-19, wherein the oculometric data from the subject is captured at about 60 frames per second or more.
22. The method of any one of aspects 1-19, wherein the oculometric data from the subject is captured at about 100 frames per second or more.
23. The method of any one of aspects 1-19, wherein the oculometric data from the subject is captured at about 200 frames per second or more.
24. The method of any one of aspects 1-23, further comprising measuring a change in one or more facial biometrics of the subject to provide facial biometrics data.
25. The method of aspect 24, further comprising performing a first order statistical analysis of the facial biometrics data.
26. The method of aspect 25, further comprising determining the presence or absence of a change relative to baseline in the first order statistical analysis of the facial biometrics data.
27. The method of any one of aspects 24-26, wherein the one or more facial biometrics comprises distance between the eyes; distance between the eyelids; width of the nose; center of the nose; depth of the eye sockets; shape of the cheekbones; length of the jawline; distance between the mouth edges; center of the mouth; and/or focal weakness.
28. The method of any one of aspects 1-27, further comprising measuring prodromal changes of the oculometric data and/or facial biometrics data.
29. The method of aspect 28, wherein the prodromal changes occur one or more days before the epileptic event.
30. The method of aspect 28, wherein the prodromal changes occur one or more hours before the epileptic event.
31. The method of aspect 28, wherein the prodromal changes occur one or more seconds before the epileptic event.
32. The method of any one of aspects 28-31, further comprising performing a first order statistical analysis of the prodromal changes of the oculometric data and/or facial biometrics data.
33. The method of aspect 32, further comprising determining the presence or absence of a change relative to baseline in the first order statistical analysis of the prodromal changes of the oculometric data and/or facial biometrics data.
34. The method of any one of aspects 1-33, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.
35. The method of any one of aspects 1-34, wherein the indicating that the epileptic event has been detected and/or predicted comprises providing an alert to the subject or a caregiver of the subject.
36. The method of any one of aspects 1-35, further comprising providing a responsive neurostimulation to the subject, wherein the responsive neurostimulation is sufficient to reduce the effect of the epileptic event, when the epileptic event is detected and/or predicted.
37. The method of any one of aspects 1-36, further comprising transmitting an electric current through the neck of the subject for which an epileptic event has been detected and/or predicted to a vagus nerve in the subject for which an epileptic event has been detected and/or predicted, wherein the electric current is sufficient to terminate the epileptic event, when the epileptic event is detected and/or predicted.
38. The method of any one of aspects 1-37, further comprising administering an effective amount of an anti-epileptic drug to the subject, when the epileptic event is detected and/or predicted.
39. The method of aspect 38, wherein the anti-epileptic drug comprises one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.
40. The method of any one of aspects 1-39, wherein measuring a change in one or more oculometric parameters of at least one eye comprises measuring a change in one or more oculometric parameters of both the left eye and the right eye.
41. The method of aspect 40, wherein the one or more oculometric parameters comprise left and right eye movements.
42. The method of aspect 40 or 41, further comprising cross-correlating oculometric data of a left eye and oculometric data of a right eye of the subject.
43. The method of any one of aspects 40-42, wherein the determining the presence or absence of the change relative to baseline in the first order statistical analysis of the oculometric data comprises determining the presence of an increase in the synchronization of eye movements between the left eye and the right eye of the subject relative to baseline.
44. The method of any one of aspects 1-43, further comprising cross-correlating the first order statistical analysis of the oculometric data.
45. The method of any one of aspects 1-43, further comprising cross-correlating the first order statistical analysis of the facial biometrics data.
46. The method of any one of aspects 1-45, further comprising performing a second order statistical analysis of the oculometric data and/or facial biometrics data.
47. The method of any one of aspects 1-46, further comprising performing a higher order statistical analysis of the oculometric data and/or facial biometrics data.
48. The method of aspect 47, wherein the higher order statistical analysis of the oculometric data and/or facial biometrics data comprises kurtosis.
49. The method of aspect 48, further comprising determining the presence or absence of a change relative to baseline in the higher order statistical analysis of the oculometric data and/or facial biometrics data.
50. The method of aspect 49, wherein the determining the presence or absence of a change relative to baseline in the higher order statistical analysis of the oculometric data and/or facial biometrics data comprises determining the presence of a change from frequency independence to inter-frequency dependence of the oculometric data and/or facial biometrics data.
51. The method of aspect 49, wherein the determining the presence or absence of a change relative to baseline in the higher order statistical analysis of the oculometric data and/or facial biometrics data comprises determining change in synchronization of the oculometric data and/or facial biometrics data.
52. The method of aspect 51, wherein the determining synchronization of the oculometric data and/or facial biometrics data comprises determining frequency synchronization of the oculometric data and/or facial biometrics data.
53. The method of aspect 52, wherein the determining frequency synchronization comprises determining synchronization of dependent and/or uncoupled frequencies of the oculometric data and/or facial biometrics data.
54. The method of aspect 49, wherein the determining the presence or absence of a change in the first order statistical analysis of the oculometric data and/or facial biometrics data comprises determining the presence of positive excess kurtosis of the oculometric data and/or facial biometrics data.
55. The method of aspect 54, wherein the determining the presence of positive excess kurtosis of the oculometric data comprises determining the presence of positive excess kurtosis of eye eccentricity.
56. The method of aspect 55, wherein the positive excess kurtosis is 10 or more.
57. The method of aspect 55, wherein the positive excess kurtosis is 15 or more.
58. The method of any one of aspects 1-57, wherein the determining step utilizes machine learning.
59. The method of any one of aspects 1-58, further comprising cross-correlating the higher order statistical analysis of the oculometric data.
60. The method of any one of aspects 1-58, further comprising cross-correlating the higher order statistical analysis of the facial biometrics data.
61. The method of any one of aspects 1-60, further comprising measuring at least one electroencephalogram signal of the subject.
62. The method of aspect 61, further comprising confirming the presence or absence of a change relative to baseline in the first order statistical analysis of the oculometric data using the at least one electroencephalogram signal.
63. The method of any one of aspects 1-62, wherein the epileptic event in the subject is detected and/or predicted in the absence of measuring an electroencephalogram signal of the subject.
64. A method of identifying and treating epilepsy in a subject, the method comprising:
   a) measuring a change in one or more oculometric parameters of at least one eye of the subject overtime using a measuring device to obtain oculometric data from the subject;
   b) performing a first order statistical analysis of the oculometric data;
   c) determining the presence or absence of a change relative to baseline in the first order statistical analysis of the oculometric data;
   d) identifying the subject as having an epileptic event and/or as at risk of an epileptic event when the determining indicates the presence or absence of a change in the first order statistical analysis of the oculometric data relative to baseline; and
   e) administering an effective amount of an anti-epileptic drug to the subject identified as having an epileptic event and/or as at risk of an epileptic event.
65. The method of aspect 64, wherein the one or more oculometric parameters comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.
66. The method of aspect 64 or 65, wherein the measuring comprises measuring a change in two or more of the oculometric parameters.
67. The method of any one of aspects 64-66, wherein eye eccentricity is a function of visible x-width and y-width of the pupil of an eye.
68. The method of aspect 67, wherein eye eccentricity changes as the eyelid position, position of the sides of the eye, pupil area, and/or blink frequency change(s).
69. The method of any one of aspects 64-68, wherein the first order statistical analysis of the oculometric data comprises multiple regression analysis and/or mean calculations of the oculometric data.
70. The method of any one of aspects 64-69, wherein the measuring device is configured to obtain oculometric data from the subject for about thirty minutes.
71. The method of any one of aspects 64-69, wherein the measuring device is configured to obtain oculometric data from the subject for about fifteen minutes.
72. The method of aspect 70 or 71, wherein the performing the first order statistical analysis of the oculometric data occurs in a ten-second running window.
73. The method of aspect 70 or 71, wherein the performing the first order statistical analysis of the oculometric data occurs in a five-second running window.
74. The method of any one of aspects 64-74, wherein the measuring device is an eye tracking device.
75. The method of aspect 74, wherein the eye tracking device comprises one or more cameras.
76. The method of aspect 75, wherein the eye tracking device further comprises a video recorder and/or a sensor.
77. The method of aspect 76, wherein the eye tracking device is a wearable device configured to be worn on the head of the subject.
78. The method of aspect 77, wherein the one or more cameras of the wearable device is located at a distance of one or more centimeters from the eyes of the subject.
79. The method of any one of aspects 64-76, wherein the eye tracking device is a contact lens.
80. The method of any one of aspects 64-79, wherein the performing the first order statistical analysis of the oculometric data comprises performing multiple regression analysis of the oculometric data.
81. The method of aspect 80, wherein the determining the presence or absence of a change in the first order statistical analysis of the oculometric data comprises determining the presence or absence of an increased correlation of one or more oculometric parameters with the epileptic event.
82. The method of aspect 81, wherein the determining the presence or absence of an increased correlation of one or more oculometric parameters with the epileptic event comprises determining the presence or absence of an increased correlation of eye eccentricity with the epileptic event.
83. The method of any one of aspects 64-82, wherein the oculometric data from the subject is captured at about 30 frames per second or more.
84. The method of any one of aspects 64-82, wherein the oculometric data from the subject is captured at about 60 frames per second or more.
85. The method of any one of aspects 64-82, wherein the oculometric data from the subject is captured at about 100 frames per second or more.
86. The method of any one of aspects 64-82, wherein the oculometric data from the subject is captured at about 200 frames per second or more.
87. The method of any one of aspects 64-86, further comprising measuring a change in one or more facial biometrics of the subject to provide facial biometrics data.
88. The method of aspect 87, further comprising performing a first order statistical analysis of the facial biometrics data.
89. The method of aspect 89, further comprising determining the presence or absence of a change relative to baseline in the first order statistical analysis of the facial biometrics data.
90. The method of any one of aspects 87-89, wherein the one or more facial biometrics comprises distance between the eyes; distance between the eyelids; width of the nose; center of the nose; depth of the eye sockets; shape of the cheekbones; length of the jawline; distance between the mouth edges; center of the mouth; and/or focal weakness.
91. The method of any one of aspects 64-90, further comprising measuring prodromal changes of the oculometric data and/or facial biometrics data.
92. The method of aspect 91, wherein the prodromal changes occur one or more days before the epileptic event.
93. The method of aspect 91, wherein the prodromal changes occur one or more hours before the epileptic event.
94. The method of aspect 91, wherein the prodromal changes occur one or more seconds before the epileptic event.
95. The method of any one of aspects 91-94, further comprising performing a first order statistical analysis of the prodromal changes of the oculometric data and/or facial biometrics data.
96. The method of aspect 95, further comprising determining the presence or absence of a change relative to baseline in the first order statistical analysis of the prodromal changes of the oculometric data and/or facial biometrics data.
97. The method of any one of aspects 64-96, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.
98. The method of any one of aspects 64-97, wherein the identifying comprises providing an alert to the subject or a caregiver of the subject.
99. The method of any one of aspects 64-98, further comprising providing a responsive neurostimulation to the subject, wherein the responsive neurostimulation is sufficient to reduce the effect of the epileptic event, when the subject is identified as having an epileptic event and/or as at risk of an epileptic event.
100. The method of any one of aspects 64-99, further comprising transmitting an electric current through the neck of the subject for which an epileptic event has been detected and/or predicted to a vagus nerve in the subject for which an epileptic event has been detected and/or predicted, wherein the electric current is sufficient to terminate the epileptic event, when the subject is identified as having an epileptic event and/or as at risk of an epileptic event.
101. The method of aspect 64, wherein the anti-epileptic drug comprises one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.
102. The method of any one of aspects 64-101, wherein measuring a change in one or more oculometric parameters of at least one eye comprises measuring a change in one or more oculometric parameters of both the left eye and the right eye.
103. The method of aspect 102, wherein the one or more oculometric parameters comprise left and right eye movements.
104. The method of aspect 102 or 103, further comprising cross-correlating oculometric data of a left eye and oculometric data of a right eye of the subject.
105. The method of any one of aspects 102-104, wherein the determining the presence or absence of the change relative to baseline in the first order statistical analysis of the oculometric data comprises determining the presence of an increase in the synchronization of eye movements between the left eye and the right eye of the subject relative to baseline.
106. The method of any one of aspects 64-105, further comprising cross-correlating the first order statistical analysis of the oculometric data.
107. The method of any one of aspects 64-105, further comprising cross-correlating the first order statistical analysis of the facial biometrics data.
108. The method of any one of aspects 64-107, further comprising performing a second order statistical analysis of the oculometric data and/or facial biometrics data.
109. The method of any one of aspects 64-108, further comprising performing a higher order statistical analysis of the oculometric data and/or facial biometrics data.
110. The method of aspect 109, wherein the higher order statistical analysis of the oculometric data and/or facial biometrics data comprises kurtosis.
111. The method of aspect 110, further comprising determining the presence or absence of a change relative to baseline in the higher order statistical analysis of the oculometric data and/or facial biometrics data.
112. The method of aspect 111, wherein the determining the presence or absence of a change relative to baseline in the higher order statistical analysis of the oculometric data and/or facial biometrics data comprises determining the presence of a change from frequency independence to inter-frequency dependence of the oculometric data and/or facial biometrics data.
113. The method of aspect 111, wherein the determining the presence or absence of a change relative to baseline in the higher order statistical analysis of the oculometric data and/or facial biometrics data comprises determining change in synchronization of the oculometric data and/or facial biometrics data.
114. The method of aspect 113, wherein the determining synchronization of the oculometric data and/or facial biometrics data comprises determining frequency synchronization of the oculometric data and/or facial biometrics data.
115. The method of aspect 114, wherein the determining frequency synchronization comprises determining synchronization of dependent and/or uncoupled frequencies of the oculometric data and/or facial biometrics data.
116. The method of aspect 111, wherein the determining the presence or absence of a change in the first order statistical analysis of the oculometric data and/or facial biometrics data comprises determining the presence of positive excess kurtosis of the oculometric data and/or facial biometrics data.
117. The method of aspect 116, wherein the determining the presence of positive excess kurtosis of the oculometric data comprises determining the presence of positive excess kurtosis of eye eccentricity.
118. The method of aspect 117, wherein the positive excess kurtosis is 10 or more.
119. The method of aspect 117, wherein the positive excess kurtosis is 15 or more.
120. The method of any one of aspects 64-119, wherein the determining step utilizes machine learning.
121. The method of any one of aspects 64-120, further comprising cross-correlating the higher order statistical analysis of the oculometric data.
122. The method of any one of aspects 64-120, further comprising cross-correlating the higher order statistical analysis of the facial biometrics data.
123. The method of any one of aspects 64-120, further comprising measuring at least one electroencephalogram signal of the subject.
124. The method of aspect 123, further comprising confirming the presence or absence of a change relative to baseline in the first order statistical analysis of the oculometric data using the at least one electroencephalogram signal.
125. The method of any one of aspects 64-124, wherein the epileptic event in the subject is detected and/or predicted in the absence of measuring an electroencephalogram signal of the subject.
126. A method of identifying and treating epilepsy in a subject, the method comprising:
   a) measuring a change in one or more oculometric parameters of at least one eye of the subject overtime using a measuring device to obtain oculometric data from the subject;
   b) performing a first order statistical analysis of the oculometric data;
   c) determining the presence or absence of a change relative to baseline in the first order statistical analysis of the oculometric data;
   d) identifying the subject as having an epileptic event and/or as at risk of an epileptic event when the determining indicates the presence or absence of a change in the first order statistical analysis of the oculometric data relative to baseline; and
   e) transmitting an electric current through the neck of the subject identified as having an epileptic event and/or as at risk of an epileptic event to a vagus nerve in the subject, wherein the electric current is sufficient to terminate the epileptic event.
127. The method of aspect 126, wherein the one or more oculometric parameters comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.
128. The method of aspect 126 or 127, wherein the measuring comprises measuring a change in two or more of the oculometric parameters.
129. The method of any one of aspects 126-128, wherein eye eccentricity is a function of visible x-width and y-width of the pupil of an eye.
130. The method of aspect 129, wherein eye eccentricity changes as the eyelid position, position of the sides of the eye, pupil area, and/or blink frequency change(s).
131. The method of any one of aspects 126-130, wherein the first order statistical analysis of the oculometric data comprises multiple regression analysis and/or mean calculations of the oculometric data.
132. The method of any one of aspects 126-131, wherein the measuring device is configured to obtain oculometric data from the subject for about thirty minutes.
133. The method of any one of aspects 126-131, wherein the measuring device is configured to obtain oculometric data from the subject for about fifteen minutes.
134. The method of aspect 132 or 133, wherein the performing the first order statistical analysis of the oculometric data occurs in a ten-second running window.
135. The method of aspect 132 or 133, wherein the performing the first order statistical analysis of the oculometric data occurs in a five-second running window.
136. The method of any one of aspects 126-135, wherein the measuring device is an eye tracking device.
137. The method of aspect 136, wherein the eye tracking device comprises one or more cameras.
138. The method of aspect 137, wherein the eye tracking device further comprises a video recorder and/or a sensor.
139. The method of aspect 138, wherein the eye tracking device is a wearable device configured to be worn on the head of the subject.
140. The method of aspect 139, wherein the one or more cameras of the wearable device is located at a distance of one or more centimeters from the eyes of the subject.
141. The method of any one of aspects 126-138, wherein the eye tracking device is a contact lens.
142. The method of any one of aspects 126-141, wherein the performing the first order statistical analysis of the oculometric data comprises performing multiple regression analysis of the oculometric data.
143. The method of aspect 142, wherein the determining the presence or absence of a change in the first order statistical analysis of the oculometric data comprises determining the presence or absence of an increased correlation of one or more oculometric parameters with the epileptic event.
144. The method of aspect 143, wherein the determining the presence or absence of an increased correlation of one or more oculometric parameters with the epileptic event comprises determining the presence or absence of an increased correlation of eye eccentricity with the epileptic event.
145. The method of any one of aspects 126-144, wherein the oculometric data from the subject is captured at about 30 frames per second or more.
146. The method of any one of aspects 126-144, wherein the oculometric data from the subject is captured at about 60 frames per second or more.
147. The method of any one of aspects 126-144, wherein the oculometric data from the subject is captured at about 100 frames per second or more.
148. The method of any one of aspects 126-144, wherein the oculometric data from the subject is captured at about 200 frames per second or more.
149. The method of any one of aspects 126-148, further comprising measuring a change in one or more facial biometrics of the subject to provide facial biometrics data.
150. The method of aspect 149, further comprising performing a first order statistical analysis of the facial biometrics data.
151. The method of aspect 150, further comprising determining the presence or absence of a change relative to baseline in the first order statistical analysis of the facial biometrics data.
152. The method of any one of aspects 149-151, wherein the one or more facial biometrics comprises distance between the eyes; distance between the eyelids; width of the nose; center of the nose; depth of the eye sockets; shape of the cheekbones; length of the jawline; distance between the mouth edges; center of the mouth; and/or focal weakness.
153. The method of any one of aspects 126-152, further comprising measuring prodromal changes of the oculometric data and/or facial biometrics data.
154. The method of aspect 153, wherein the prodromal changes occur one or more days before the epileptic event.
155. The method of aspect 153, wherein the prodromal changes occur one or more hours before the epileptic event.
156. The method of aspect 153, wherein the prodromal changes occur one or more seconds before the epileptic event.
157. The method of any one of aspects 153-156, further comprising performing a first order statistical analysis of the prodromal changes of the oculometric data and/or facial biometrics data.
158. The method of aspect 157, further comprising determining the presence or absence of a change relative to baseline in the first order statistical analysis of the prodromal changes of the oculometric data and/or facial biometrics data.
159. The method of any one of aspects 126-158, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.
160. The method of any one of aspects 126-159, wherein the identifying comprises providing an alert to the subject or a caregiver of the subject.
161. The method of any one of aspects 126-160, wherein measuring a change in one or more oculometric parameters of at least one eye comprises measuring a change in one or more oculometric parameters of both the left eye and the right eye.
162. The method of aspect 161, wherein the one or more oculometric parameters comprise left and right eye movements.
163. The method of aspect 161 or 162, further comprising cross-correlating oculometric data of a left eye and oculometric data of a right eye of the subject.
164. The method of any one of aspects 161-163, wherein the determining the presence or absence of the change relative to baseline in the first order statistical analysis of the oculometric data comprises determining the presence of an increase in the synchronization of eye movements between the left eye and the right eye of the subject relative to baseline.
165. The method of any one of aspects 126-164, further comprising cross-correlating the first order statistical analysis of the oculometric data.
166. The method of any one of aspects 126-164, further comprising cross-correlating the first order statistical analysis of the facial biometrics data.
167. The method of any one of aspects 1-166, further comprising performing a second order statistical analysis of the oculometric data and/or facial biometrics data.
168. The method of any one of aspects 126-164, further comprising performing a higher order statistical analysis of the oculometric data and/or facial biometrics data.
169. The method of aspect 168, wherein the higher order statistical analysis of the oculometric data and/or facial biometrics data comprises kurtosis.
170. The method of aspect 169, further comprising determining the presence or absence of a change relative to baseline in the higher order statistical analysis of the oculometric data and/or facial biometrics data.
171. The method of aspect 170, wherein the determining the presence or absence of a change relative to baseline in the higher order statistical analysis of the oculometric data and/or facial biometrics data comprises determining the presence of a change from frequency independence to inter-frequency dependence of the oculometric data and/or facial biometrics data.
172. The method of aspect 170, wherein the determining the presence or absence of a change relative to baseline in the higher order statistical analysis of the oculometric data and/or facial biometrics data comprises determining change in synchronization of the oculometric data and/or facial biometrics data.
173. The method of aspect 172, wherein the determining synchronization of the oculometric data and/or facial biometrics data comprises determining frequency synchronization of the oculometric data and/or facial biometrics data.
174. The method of aspect 173, wherein the determining frequency synchronization comprises determining synchronization of dependent and/or uncoupled frequencies of the oculometric data and/or facial biometrics data.
175. The method of aspect 170, wherein the determining the presence or absence of a change in the first order statistical analysis of the oculometric data and/or facial biometrics data comprises determining the presence of positive excess kurtosis of the oculometric data and/or facial biometrics data.
176. The method of aspect 175, wherein the determining the presence of positive excess kurtosis of the oculometric data comprises determining the presence of positive excess kurtosis of eye eccentricity.
177. The method of aspect 176, wherein the positive excess kurtosis is 10 or more.
178. The method of aspect 176, wherein the positive excess kurtosis is 15 or more.
179. The method of any one of aspects 126-178, wherein the determining step utilizes machine learning.
180. The method of any one of aspects 126-179, further comprising cross-correlating the higher order statistical analysis of the oculometric data.
181. The method of any one of aspects 126-179, further comprising cross-correlating the higher order statistical analysis of the facial biometrics data.
182. The method of any one of aspects 126-179, further comprising measuring at least one electroencephalogram signal of the subject.
183. The method of aspect 182, further comprising confirming the presence or absence of a change relative to baseline in the first order statistical analysis of the oculometric data using the at least one electroencephalogram signal.
184. The method of any one of aspects 126-183, wherein the epileptic event in the subject is detected and/or predicted in the absence of measuring an electroencephalogram signal of the subject.
185. A method of detecting and/or predicting an epileptic event in a subject, said method comprising:
   a) measuring left and right eye movements over time using a measuring device to obtain eye movement data from the subject;
   b) identifying the presence or absence of an increase in the correlation of left and right eye movements over time based on the measuring; and
   c) indicating that an epileptic seizure has been detected and/or predicted when the identifying indicates the presence of an increase in the correlation of left and right eye movements over time.
186. The method of aspect 185, wherein the one or more eye movements comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.
187. The method of aspect 185 or 186, wherein the measuring comprises measuring a change in two or more of the eye movements.
188. The method of any one of aspects 185-187, wherein eye eccentricity is a function of visible x-width and y-width of the pupil of an eye.
189. The method of aspect 188, wherein eye eccentricity changes as the eyelid position, position of the sides of the eye, pupil area, and/or blink frequency change(s).
190. The method of any one of aspects 185-189, wherein the measuring device is configured to obtain eye movement data from the subject for about thirty minutes.
191. The method of any one of aspects 185-189, wherein the measuring device is configured to obtain eye movement data from the subject for about fifteen minutes.
192. The method of aspect 190 or 191, wherein the performing the first order statistical analysis of the eye movement data occurs in a ten-second running window.
193. The method of aspect 190 or 191, wherein the performing the first order statistical analysis of the eye movement data occurs in a five-second running window.
194. The method of any one of aspects 185-193, wherein the measuring device is an eye tracking device.
195. The method of aspect 194, wherein the eye tracking device comprises one or more cameras.
196. The method of aspect 195, wherein the eye tracking device further comprises a video recorder and/or a sensor.
197. The method of aspect 196, wherein the eye tracking device is a wearable device configured to be worn on the head of the subject.
198. The method of aspect 197, wherein the one or more cameras of the wearable device is located at a distance of one or more centimeters from the eyes of the subject.
199. The method of any one of aspects 185-196, wherein the eye tracking device is a contact lens.
200. The method of any one of aspects 185-199, wherein the eye movement data from the subject is captured at about 30 frames per second or more.
201. The method of any one of aspects 185-199, wherein the eye movement data from the subject is captured at about 60 frames per second or more.
202. The method of any one of aspects 185-199, wherein the eye movement data from the subject is captured at about 100 frames per second or more.
203. The method of any one of aspects 185-199, wherein the eye movement data from the subject is captured at about 200 frames per second or more.
204. The method of any one of aspects 185-203, further comprising measuring prodromal changes of the eye movement data.
205. The method of aspect 204, wherein the prodromal changes occur one or more days before the epileptic event.
206. The method of aspect 204, wherein the prodromal changes occur one or more hours before the epileptic event.
207. The method of aspect 204, wherein the prodromal changes occur one or more seconds before the epileptic event.
208. The method of any one of aspects 185-207, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.
209. The method of any one of aspects 185-208, wherein the indicating that the epileptic event has been detected and/or predicted comprises providing an alert to the subject or a caregiver of the subject.
210. The method of any one of aspects 185-209, further comprising providing a responsive neurostimulation to the subject, wherein the responsive neurostimulation is sufficient to reduce the effect of the epileptic event, when the epileptic event is detected and/or predicted.
211. The method of any one of aspects 185-210, further comprising transmitting an electric current through the neck of the subject for which an epileptic event has been detected and/or predicted to a vagus nerve in the subject for which an epileptic event has been detected and/or predicted, wherein the electric current is sufficient to terminate the epileptic event, when the epileptic event is detected and/or predicted.
212. The method of any one of aspects 185-211, further comprising administering an effective amount of an anti-epileptic drug to the subject, when the epileptic event is detected and/or predicted.
213. The method of aspect 212, wherein the anti-epileptic drug comprises one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.
214. The method of any one of aspects 185-213, further comprising cross-correlating eye movement data of a left eye and eye movement data of a right eye of the subject.
215. The method of any one of aspects 185-214, further comprising measuring at least one electroencephalogram signal of the subject.
216. The method of aspect 215, further comprising confirming the presence or absence of a change relative to baseline in the first order statistical analysis of the eye movement data using the at least one electroencephalogram signal.
217. The method of any one of aspects 185-216, wherein the epileptic event in the subject is detected and/or predicted in the absence of measuring an electroencephalogram signal of the subject.
218. A system for detecting and/or predicting an epileptic event in a subject, the system comprising:
   a) a measuring device configured to measure a change in one or more oculometric parameters of at least one eye of the subject over time;
   b) a processor unit;
   c) a non-transitory computer-readable storage medium comprising instructions, which when executed by the processor unit, cause the processor unit to perform a first order statistical analysis of the oculometric data and determine the presence or absence of a change relative to baseline in the first order statistical analysis of the oculometric data; and
   c) an output device configured to indicate that an epileptic event has been detected and/or predicted when a change in the first order statistical analysis is determined to be present.
219. The system of aspect 218, wherein the one or more oculometric parameters comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.
220. The system of aspect 218 or 219, wherein the measuring device measures a change in two or more of the oculometric parameters.
221. The system of any one of aspects 218-220, wherein eye eccentricity is a function of visible x-width and y-width of the pupil of an eye.
222. The system of aspect 221, wherein eye eccentricity changes as the eyelid position, position of the sides of the eye, pupil area, and/or blink frequency change(s).
223. The system of any one of aspects 218-222, wherein the first order statistical analysis of the oculometric data comprises multiple regression analysis and/or mean calculations of the oculometric data.
224. The system of any one of aspects 218-223, wherein the measuring device is configured to obtain oculometric data from the subject for about thirty minutes.
225. The system of any one of aspects 218-223, wherein the measuring device is configured to obtain oculometric data from the subject for about fifteen minutes.
226. The system of aspect 224 or 225, wherein the non-transitory computer-readable storage medium comprises instructions, which when executed by the processor unit, cause the processor unit to perform the first order statistical analysis of the oculometric data in a ten-second running window.
227. The system of aspect 224 or 225, wherein the non-transitory computer-readable storage medium comprises instructions, which when executed by the processor unit, cause the processor unit to perform the first order statistical analysis of the oculometric data in a five-second running window.
228. The system of any one of aspects 218-227, wherein the measuring device is an eye tracking device.
229. The system of aspect 228, wherein the eye tracking device comprises one or more cameras.
230. The system of aspect 229, wherein the eye tracking device further comprises a video recorder and/or a sensor.
231. The system of aspect 230, wherein the eye tracking device is a wearable device configured to be worn on the head of the subject.
232. The system of aspect 231, wherein the one or more cameras of the wearable device is located at a distance of one or more centimeters from the eyes of the subject.
233. The system of any one of aspects 218-230, wherein the eye tracking device is a contact lens.
234. The system of any one of aspects 218-233, wherein the non-transitory computer-readable storage medium comprising instructions, which when executed by the processor unit, cause the processor unit to perform the first order statistical analysis of the oculometric data comprises performing multiple regression analysis of the oculometric data.
235. The system of aspect 234, wherein determining the presence or absence of a change in the first order statistical analysis of the oculometric data comprises determining the presence or absence of an increased correlation of one or more oculometric parameters with the epileptic event.
236. The system of aspect 235, wherein determining the presence or absence of an increased correlation of one or more oculometric parameters with the epileptic event comprises determining the presence or absence of an increased correlation of eye eccentricity with the epileptic event.
237. The system of any one of aspects 218-236, wherein the oculometric data from the subject is captured at about 30 frames per second or more.
238. The system of any one of aspects 218-236, wherein the oculometric data from the subject is captured at about 60 frames per second or more.
239. The system of any one of aspects 218-236, wherein the oculometric data from the subject is captured at about 100 frames per second or more.
240. The system of any one of aspects 218-236, wherein the oculometric data from the subject is captured at about 200 frames per second or more.
241. The system of any one of aspects 218-240, wherein the measuring device is further configured to measure a change in one or more facial biometrics of the subject to provide facial biometrics data.
242. The system of aspect 241, wherein the non-transitory computer readable storage medium further comprises instructions, which when executed by the processor unit, cause the processor unit to perform a first order statistical analysis of the facial biometrics data.
243. The system of aspect 242, wherein the non-transitory computer readable storage medium further comprises instructions, which when executed by the processor unit, cause the processor unit to determine the presence or absence of a change relative to baseline in the first order statistical analysis of the facial biometrics data.
244. The system of any one of aspects 241-243, wherein the one or more facial biometrics comprises distance between the eyes; distance between the eyelids; width of the nose; center of the nose; depth of the eye sockets; shape of the cheekbones; length of the jawline; distance between the mouth edges; center of the mouth; and/or focal weakness.
245. The system of any one of aspects 218-244, wherein the measuring device is further configured to measure prodromal changes of the oculometric data and/or facial biometrics data.
246. The system of aspect 245, wherein the prodromal changes occur one or more days before the epileptic event.
247. The system of aspect 245, wherein the prodromal changes occur one or more hours before the epileptic event.
248. The system of aspect 245, wherein the prodromal changes occur one or more seconds before the epileptic event.
249. The system of any one of aspects 245-248, wherein the non-transitory computer readable storage medium further comprises instructions, which when executed by the processor unit, cause the processor unit to perform a first order statistical analysis of the prodromal changes of the oculometric data and/or facial biometrics data.
250. The system of aspect 249, wherein the non-transitory computer readable storage medium further comprises instructions, which when executed by the processor unit, cause the processor unit to determine the presence or absence of a change relative to baseline in the first order statistical analysis of the prodromal changes of the oculometric data and/or facial biometrics data.
251. The system of any one of aspects 218-250, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.
252. The system of any one of aspects 218-251, wherein the output device configured to indicate that the epileptic event has been detected and/or predicted comprises providing an alert to the subject or a caregiver of the subject.
253. The system of aspect any one of aspects 218-252, further comprising a neurostimulation device configured to provide a responsive neurostimulation to the subject, wherein the responsive neurostimulation is sufficient to reduce the effect of the epileptic event, when the epileptic event is detected and/or predicted.
254. The system of aspect any one of aspects 218-253, further comprising a neurostimulation device configured to provide an electric current through the neck of the subject for which an epileptic event has been detected and/or predicted to a vagus nerve in the subject for which an epileptic event has been detected and/or predicted, wherein the electric current is sufficient to terminate the epileptic event, when the epileptic event is detected and/or predicted.
255. The system of any one of aspects 218-254, further comprising a drug administration device configured to administer an effective amount of an anti-epileptic drug to the subject, when the epileptic event is detected and/or predicted.
256. The system of aspect 255, wherein the anti-epileptic drug comprises one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.
257. The system of any one of aspects 218-256, wherein measuring a change in one or more oculometric parameters of at least one eye comprises measuring a change in one or more oculometric parameters of both the left eye and the right eye.
258. The system of aspect 257, wherein the one or more oculometric parameters comprise left and right eye movements.
259. The system of aspect 257 or 258, wherein the non-transitory computer-readable storage medium comprises instructions, which when executed by the processor unit, cause the processor unit to cross-correlate oculometric data of a left eye and oculometric data of a right eye of the subject.
260. The system of any one of aspects 257-259, wherein determining the presence or absence of the change relative to baseline in the first order statistical analysis of the oculometric data comprises determining the presence of an increase in the synchronization of eye movements between the left eye and the right eye of the subject relative to baseline.
261. The system of any one of aspects 218-260, wherein the non-transitory computer-readable storage medium comprises instructions, which when executed by the processor unit, cause the processor unit to cross-correlate the first order statistical analysis of the oculometric data.
262. The system of any one of aspects 218-260, wherein the non-transitory computer-readable storage medium comprises instructions, which when executed by the processor unit, cause the processor unit to cross-correlate the first order statistical analysis of the facial biometrics data.
263. The system of any one of aspects 218-262, wherein the non-transitory computer-readable storage medium comprises instructions, which when executed by the processor unit, cause the processor unit to perform a second order statistical analysis of the oculometric data and/or facial biometrics data.
264. The system of any one of aspects 218-260, wherein the non-transitory computer readable storage medium further comprises instructions, which when executed by the processor unit, cause the processor unit to perform a higher order statistical analysis of the oculometric data and/or facial biometrics data.
265. The system of aspect 264, wherein the higher order statistical analysis of the oculometric data and/or facial biometrics data comprises kurtosis.
266. The system of aspect 265, wherein the non-transitory computer readable storage medium further comprises instructions, which when executed by the processor unit, cause the processor unit to determine the presence or absence of a change relative to baseline in the higher order statistical analysis of the oculometric data and/or facial biometrics data.
267. The system of aspect 266, wherein determining the presence or absence of a change relative to baseline in the higher order statistical analysis of the oculometric data and/or facial biometrics data comprises determining the presence of a change from frequency independence to inter-frequency dependence of the oculometric data and/or facial biometrics data.
268. The system of aspect 266, wherein determining the presence or absence of a change relative to baseline in the higher order statistical analysis of the oculometric data and/or facial biometrics data comprises determining change in synchronization of the oculometric data and/or facial biometrics data.
269. The system of aspect 268, wherein determining synchronization of the oculometric data and/or facial biometrics data comprises determining frequency synchronization of the oculometric data and/or facial biometrics data.
270. The system of aspect 269, wherein determining frequency synchronization comprises determining synchronization of dependent and/or uncoupled frequencies of the oculometric data and/or facial biometrics data.
271. The system of aspect 266, wherein determining the presence or absence of a change in the first order statistical analysis of the oculometric data and/or facial biometrics data comprises determining the presence of positive excess kurtosis of the oculometric data and/or facial biometrics data.
272. The system of aspect 271, wherein determining the presence of positive excess kurtosis of the oculometric data comprises determining the presence of positive excess kurtosis of eye eccentricity.
273. The system of aspect 272, wherein the positive excess kurtosis is 10 or more.
274. The system of aspect 272, wherein the positive excess kurtosis is 15 or more.
275. The system of any one of aspects 218-274, wherein the system is aided by machine learning.
276. The system of any one of aspects 218-275, wherein the non-transitory computer-readable storage medium comprising instructions, which when executed by the processor unit, cause the processor unit to cross-correlate the higher order statistical analysis of the oculometric data.
277. The system of any one of aspects 218-275, wherein the non-transitory computer-readable storage medium comprises instructions, which when executed by the processor unit, cause the processor unit to cross-correlate the higher order statistical analysis of the facial biometrics data.
278. The system of any one of aspects 218-275, wherein the processor unit comprises a memory field for containing a computer interface.
279. The system of any one of aspects 218-278, wherein the output device comprises a memory field for containing a computer interface.
280. The system of any one of aspects 218-279, further comprising an input device configured to measure at least one electroencephalogram signal on the subject.
281. The system of aspect 280, wherein the non-transitory computer-readable storage medium comprises instructions, which when executed by the processor unit, cause the processor unit to confirm the presence or absence of a change relative to baseline in the first order statistical analysis of the oculometric data using the at least one electroencephalogram signal.
282. The system of any one of aspects 218-281, wherein the epileptic event in the subject is detected and/or predicted in the absence of measuring an electroencephalogram signal of the subject.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods

The following materials and methods generally apply to the results presented in the Examples described herein except where noted otherwise.

### Experimental Design

The experiments were performed with the approval of Children's Hospital and Research Center Oakland (CHO) Institutional Review Board on consecutive children referred for routine EEGs in CHO neurophysiology lab. The patients were screened for risk factors for seizures likely to be captured during a routine EEG such as history of staring spells or known absence epilepsy. Patients were excluded from the study if they had a history of aggression or it was anticipated that they would have difficulty tolerating the EEG. Patients and their families had the study explained to them by the CHO study coordinator or principal investigator. After the patient consented, a routine EEG was performed with the addition of an Eye-Com Biosensor™ Model EC-7T device worn during the EEG session like a pair of glasses.

Nihon-Kohden EEG acquisition and reading software were used. The acquisition platform was modified to allow the output of the Eye-Com Biosensor™ Model EC-7T system to be collected and displayed simultaneously. The data collection from the Eye-Com Biosensor™ and Nihon-Kohden EEG were synchronized into a single compatible platform for data collection and analysis. The Eye-Com Biosensor™ device was physically adapted to use on children undergoing EEG.

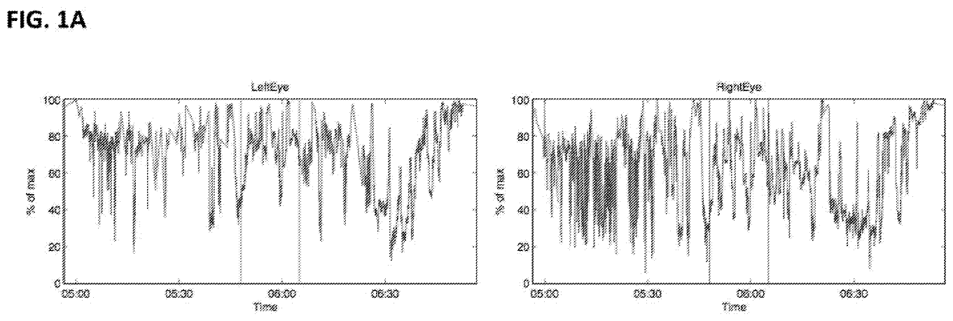

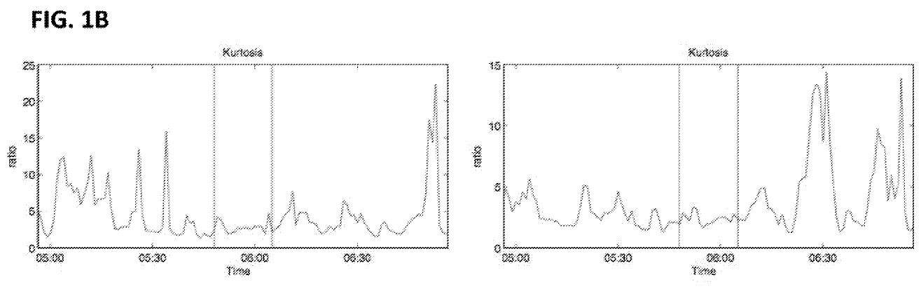

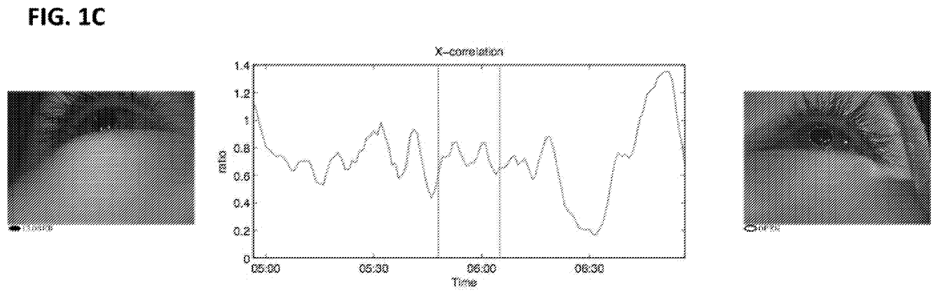

A total of 30 patients were enrolled. Six patients had a total of 24 electro-clinical seizures. Nine patients had normal EEGs. Fifteen patients had abnormal EEGs without clinical seizures captured. The results of the EEG data are depicted in FIGS. 1A-10C.

Devices for Measuring a Change in One or More Oculometric Parameters and/or Facial Biometric Parameters

The experiments used the Eye-Com Biosensor™, an eye-tracking platform that used frame-mounted micro-cameras recording video of the eye at 30 fps. The micro-cameras were located in an eye-frame at a distance of 1 to 2 centimeters from the eyes of the subjects. The Eye-Com™ software translated the video into dynamic continuous ocular measures, before, during and after an epileptic event. The Eye-Com Biosensor™ captured and recorded over 20 different oculometric parameters, including, but not limited to, pupil area to iris area ratio, pupil constriction/dilation rate and velocity, pupil diameter, saccadic and torsional velocity and direction, eye blink rate and duration, all of which were monitored in real time in synchrony with the EEG data and video of the eyes and body of the subject. The resolution of the face and body video obtained during the EEG was insufficient for facial biometric analysis. In some prophetic embodiments, a goggle with cameras mounted inside for sleeping may be used or a camera mounted on a hat may be used. In other aspects, a video camera capturing images at a rate of more than 200 fps, positioned close to the eyes and face and mounted on the head, wall, or ears may be used.

In some prophetic embodiments, one or more oculometric parameters may be measured inside the eye using a contact lens. In the conducted experiments, oculometric data was difficult to obtain with the eyes closed. The outline of the iris and pupil could be seen through the eyelid but was not as sensitive as data measured under the eyelid directly over the eye. Exemplary contact lens systems that may be used in prophetic examples include, but are not limited to, those known in the art, such as the system disclosed in US 20170049395, the disclosure of which is incorporated herein by reference. Other exemplary devices that may be used in prophetic examples include, but are not limited to Google Glass™ and/or Pupil Lab Pupil™.

### Performing Statistical Analysis

Statistical analysis was performed using MATLAB. MATLAB is a proprietary programming language developed by MathWorks® that allows matrix manipulations, plotting of functions and data, implementation of algorithms, creation of user interfaces, and interfacing with programs written in other languages, including C, C++, C#, Java, Fortran and Python. Exemplary toolboxes within MATLAB include, but are not limited to, Statistics and Machine Learning Toolbox™, Neural Network Toolbox™, Image Processing Toolbox™, Image Acquisition Toolbox™, and Mapping Toolbox™.

In some cases, statistical analysis was performed using MATLAB. In other cases, MATLAB's Computer Vision System Toolbox™ may be utilized, which provides algorithms, functions, and apps for designing and simulating computer vision and video processing systems. For 3D computer vision, the system toolbox supports single, stereo, and fisheye camera calibration; stereo vision; 3D reconstruction; and 3D point cloud processing. All processes may be aided by machine learning in prophetic examples.

In some other cases, OpenCV (Open Source Computer Vision Library) may be used. OpenCV was designed for computational efficiency and with a strong focus on real-time applications. Open-source toolkits for machine vision systems were available online at the OpenCV Organization.

### Example 1

### Results

The EEG data of six subjects was analyzed in routine clinical fashion, oculometric data was collected, and seizures were time-stamped and listed below in Table 1.

TABLE 1
EEG and oculometric data of six subjects identifying the epileptic event,
time-stamp recorded by EyeCom Biosensor ™ and by a Nihon Kohden
EEG monitoring system, along with the duration of the epileptic event.

| TIMELINES: | | | | | |
|---|---|---|---|---|---|
| ID | # | EVENT | EYECOM ™ | N-K EEG | EEG |
| 060313 | | START | 00:00:00 | 00:00:23 | |
| | 1 | CLIN SEIZURE ONSET | 00:06:43 | 00:07:06 | 9 sec, gen 3 HZ S + W |
| | | | | | (spike + wave) |
| | | SEIZURE ENDS | 00:06:54 | 00:07:17 | |
| 060713 | | START | 00:00:00 | 00:00:14 | |
| | 1 | CLIN SEIZURE ONSET | 00:05:48 | 00:06:02 | 3-4 HZ S + W |
| | | SEIZURE ENDS | 00:06:05 | 00:06:19 | |
| | 2 | CLIN SEIZURE ONSET | 00:06:45 | 00:06:59 | 3-4 HZ S + W |
| | | SEIZURE ENDS | 00:06:58 | 00:07:12 | |
| | 3 | POSS SZ/EYES CLOSED | 00:10:50 | 00:11:04 | 3-4 HZ S + W |
| | | POSS SZ ENDS | 00:10:56 | 00:11:10 | |
| 072513 | | START | 00:00:00 | 00:00:27 | |
| | 1 | CLIN SEIZURE ONSET | 00:00:08 | 00:00:35 | 2.5 HZ S + W |
| | | SEIZURE ENDS | 00:00:15 | 00:00:42 | |
| | 2 | CLIN SEIZURE ONSET | 00:05:25 | 00:05:52 | 2.5-3 HZ S + W |
| | | SEIZURE ENDS | 00:05:35 | 00:06:02 | |
| | 3 | CLIN SEIZURE ONSET | 00:06:37 | 00:07:04 | 2.5 HZ S + W |
| | | SEIZURE ENDS | 00:06:49 | 00:07:16 | |
| | 4 | CLIN SEIZURE ONSET | 00:12:16 | 00:12:43 | 3 HZ S + W |
| | | SEIZURE ENDS | 00:12:25 | 00:12:52 | |
| | 5 | SC SEIZURE ONSET | 00:14:41 | 00:15:08 | 3 HZ S + W |
| | | SEIZURE ENDS | 00:14:47 | 00:15:14 | |
| 080713 | | START | 00:00:00 | 00:00:15 | |
| | 1 | CLIN SEIZURE ONSET | 00:01:43 | 00:01:58 | 2.5-3 HZ S + W |
| | | SEIZURE ENDS | 00:02:07 | 00:02:22 | |
| | 2 | CLIN SEIZURE ONSET | 00:02:28 | 00:02:43 | 3 HZ S + W |
| | | SEIZURE ENDS | 00:02:48 | 00:03:03 | |
| 082313A | | START | 00:00:00 | 00:00:22 | |
| | 1 | CLIN SEIZURE ONSET | 00:00:22 | 00:00:44 | 3 HZ S + W |
| | | SEIZURE ENDS | 00:00:42 | 00:01:04 | |
| | 2 | CLIN SEIZURE ONSET | 00:08:35 | 00:08:57 | 3 HZ S + W |
| | | SEIZURE ENDS | 00:08:51 | 00:09:13 | |
| 082313B | | START | 00:00:00 | 00:00:23 | |
| | 1 | CLIN SEIZURE ONSET | 00:00:58 | 00:01:21 | 3 HZ S + W |
| | | SEIZURE ENDS | 00:01:02 | 00:01:25 | |
| | 2 | CLIN SEIZURE ONSET | 00:06:34 | 00:07:07 | 2.5 HZ S + W |
| | | SEIZURE ENDS | 00:07:00 | 00:07:23 | |
| | 3 | CLIN SEIZURE ONSET | 00:07:31 | 00:07:54 | 3 HZ S + W |
| | | SEIZURE ENDS | 00:07:46 | 00:08:09 | |
| | 4 | CLIN SEIZURE ONSET | 00:08:08 | 00:08:31 | 2.5 HZ S + W |
| | | SEIZURE ENDS | 00:08:19 | 00:08:42 | |
| | 5 | CLIN SEIZURE ONSET | 00:08:47 | 00:09:10 | 2.5 HZ S + W |
| | | SEIZURE ENDS | 00:08:56 | 00:09:19 | |
| | 6 | CLIN SEIZURE ONSET | 00:09:40 | 00:10:03 | 2.7 HZ S + W |
| | | SEIZURE ENDS | 00:09:45 | 00:10:08 | |
| | 7 | CLIN SEIZURE ONSET | 00:14:17 | 00:14:40 | 2.5 HZ S + W |
| | | SEIZURE ENDS | 00:14:20 | 00:14:43 | |
| | 8 | CLIN SEIZURE ONSET | 00:21:24 | 00:21:47 | 2.5 HZ S + W |
| | | SEIZURE ENDS | 00:21:30 | 00:21:53 | |
| | 9 | CLIN SEIZURE ONSET | 00:24:53 | 00:25:16 | 2.7 HZ S + W |
| | | SEIZURE ENDS | 00:24:56 | 00:25:19 | |
| | 10 | SEIZURE ONSET | 00:21:41 | 00:30:04 | 2.5 HZ S + W |
| | | SEIZURE ENDS | 00:21:47 | 00:30:10 | |
| | 11 | SEIZURE ONSET | 00:31:22 | 00:34:45 | 2.7 HZ S + W |
| | | SEIZURE ENDS | 00:31:27 | 00:31:50 | |
| | 12 | SEIZURE ONSET | 00:34:47 | 00:35:10 | 2.5 HZ S + W |
| | | SEIZURE ENDS | 00:34:51 | 00:35:14 | |

The types of epileptic events analyzed in Table 1 were generalized seizures, organized by subject. The epileptic event denoted when seizure began and when it ended. The experiments also captured when the subject when to sleep. During a generalized seizure, there was a loss of normal eye movements that could be measured with oculometrics and utilized in a seizure alarm. The whole brain was seizing at once during a generalized seizure which produced synchronous activity between the eyes as seen from the patients above. However, there are other seizure types including partial seizures which would likely yield a different oculometric signature. For example, there may be a change of synchronization of eye movement, or significant difference in the kurtosis of eye eccentricity. Table 1 was performed using data captured at 30 fps, thus capturing gross eye movements. In order to capture faster eye movements known as saccades, and micro-saccades an increased sampling rate may be utilized. Without intending to be bound by any particular theory, in such cases, the kurtosis change may be even greater with not just large eye movements captured but also with smaller or saccadic eye movements captured using faster cameras.

FIGS. 1A-1C depict the analysis of oculometric data derived from a subject experiencing a first epileptic event. FIG. 1A shows eye eccentricity as percent of max plotted against time for the left eye (FIG. 1A, left) and right eye (FIG. 1A, right). Since eye eccentricity represented a ratio of apparent x width and y width, such a parameter represented variability in both the pupil dilation and eye movement. The Eye-Com Biosensor™ identified an epileptic event occurring at the 5:48 time stamp. The data was confirmed using the data from an EEG. The seizure lasted for several seconds at a 3-4 Hz frequency.

FIG. 1B shows kurtosis over time in the left eye (FIG. 1B, left) and right eye (FIG. 1B, right). FIG. 1C shows the cross-correlation of eccentricity between the left eye and the right eye thus depicting the in-sync behavior of the eyes during and after an epileptic event (FIG. 1C, center). Photographs of the left eye (FIG. 1C, left) and right eye (FIG. 1C, right) during the epileptic event are also provided. The vertical red bars on the graphs denote the occurrence of an epileptic event. There appears to be no visible change for the eye movements around the time of the seizure.

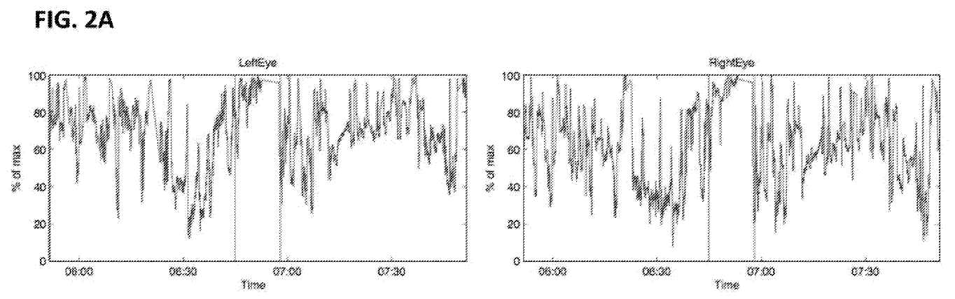

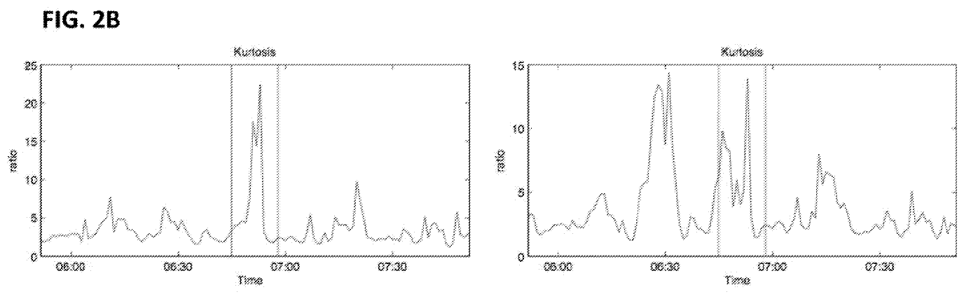

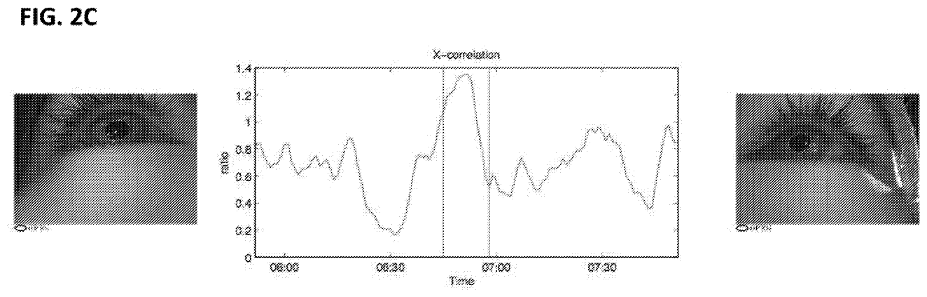

FIGS. 2A-2C depict the analysis of oculometric data derived from the same subject as in FIGS. 1A-1C experiencing a different epileptic event. FIG. 2A shows eye eccentricity as percent of max plotted against time for the left eye (FIG. 2A, left) and right eye (FIG. 2A, right). The Eye-Com Biosensor™ identified an epileptic event occurring at the 6:45 time stamp. The data was confirmed using the data from an EEG. The seizure lasted for several seconds at a 3-4 Hz frequency.

FIG. 2B shows kurtosis over time in the left eye (FIG. 2B, left) and right eye (FIG. 2B, right). Kurtosis is graphed over time in each eye from normal baseline of 3 to 20, which is consistent with a stable distribution of eye eccentricity. The seizure causes both eyes to stop moving, producing a spike in kurtosis, and a higher cross-correlation as well. FIG. 2C shows the cross-correlation of eccentricity between the left eye and the right eye (FIG. 2C, center). Photographs of the left eye (FIG. 2C, left) and right eye (FIG. 2C, right) during the epileptic event are also provided. The vertical red bars on the graphs denote the occurrence of an epileptic event.

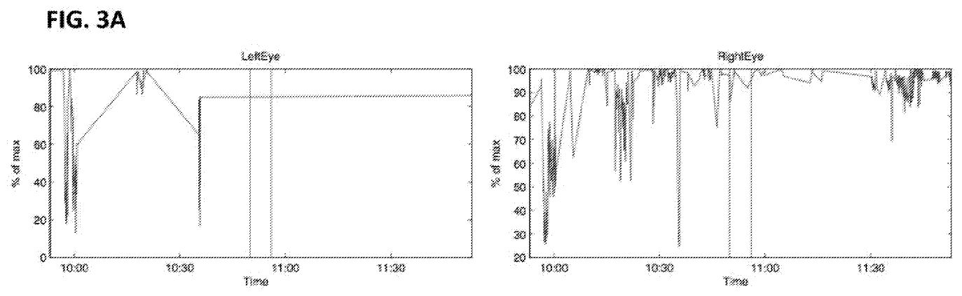

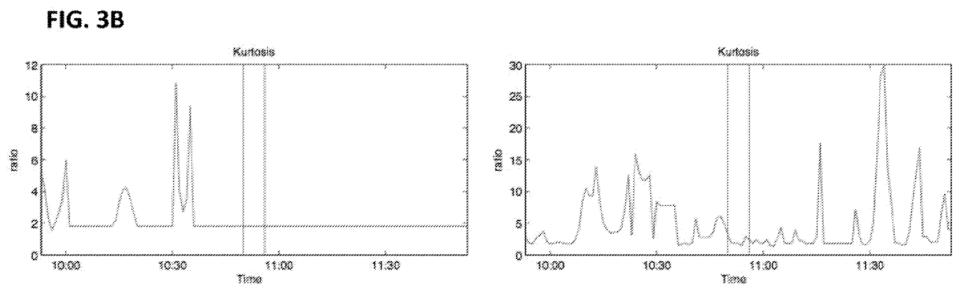

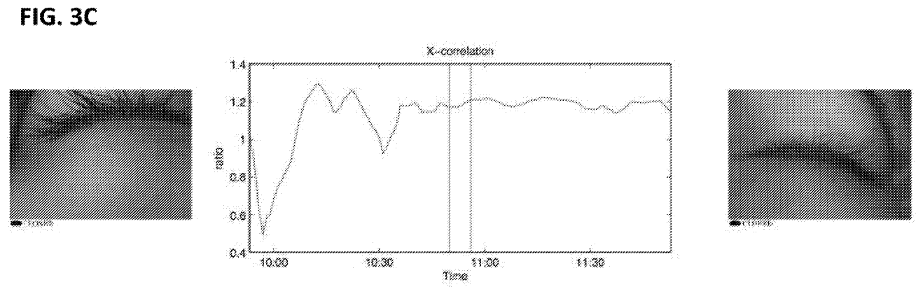

FIGS. 3A-3C depict the analysis of oculometric data derived from the same subject as in FIGS. 1A-1C and 2A-2C having closed eyes during a different epileptic event. FIG. 3A shows eye eccentricity as percent of max plotted against time for the left eye (FIG. 3A, left) and right eye (FIG. 3A, right). The Eye-Com Biosensor™ identified an epileptic event occurring at the 10:50 time stamp. However, the subjects' eyes were closed during the recorded event. The data was confirmed using the data from an EEG. The seizure was recorded at a 3-4 Hz frequency; however, no useable data was available to process.

FIG. 3B shows kurtosis over time in the left eye (FIG. 3B, left) and right eye (FIG. 3B, right). FIG. 3C shows the cross-correlation of eccentricity between the left eye and the right eye (FIG. 3C, center). Photographs of the closed left eye (FIG. 3C, left) and right eye (FIG. 3C, right) during the epileptic event are also provided. Although the vertical red bars on the graphs denote the occurrence of an epileptic event, the oculometric data received was not available for processing.

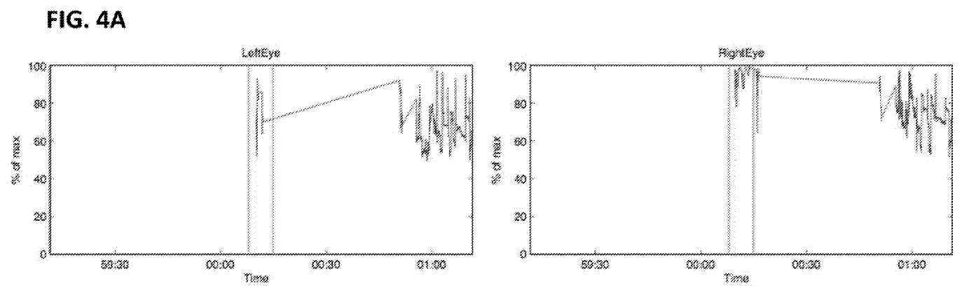

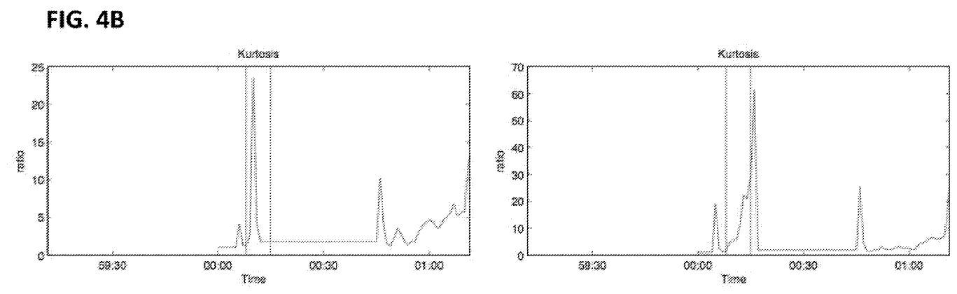

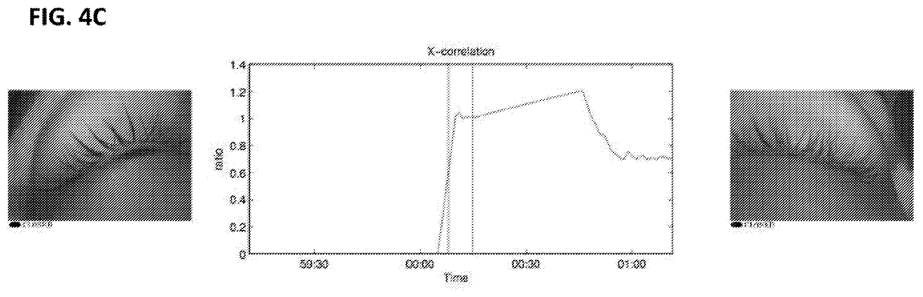

FIGS. 4A-4C depict the analysis of oculometric data derived from a subject experiencing an epileptic event at the beginning of the record. FIG. 4A shows eye eccentricity as percent of max plotted against time for the left eye (FIG. 4A, left) and right eye (FIG. 4A, right). The Eye-Com Biosensor™ identified an epileptic event occurring at the 0:08 time stamp. The data was confirmed using the data from an EEG. The seizure lasted for several seconds at a 2.5 Hz frequency.

FIG. 4B shows kurtosis over time in the left eye (FIG. 4B, left) and right eye (FIG. 4B, right). Though the seizure shows a spike in kurtosis, there is not enough data prior to the event to consider the spike a significant result. FIG. 4C shows the cross-correlation of eccentricity between the left eye and the right eye (FIG. 4C, center). Photographs of the occluded left eye (FIG. 4C, left) and right eye (FIG. 4C, right) during the epileptic event are also provided. The eyes appear closed, but are merely occluded. The vertical red bars on the graphs denote the occurrence of an epileptic event.

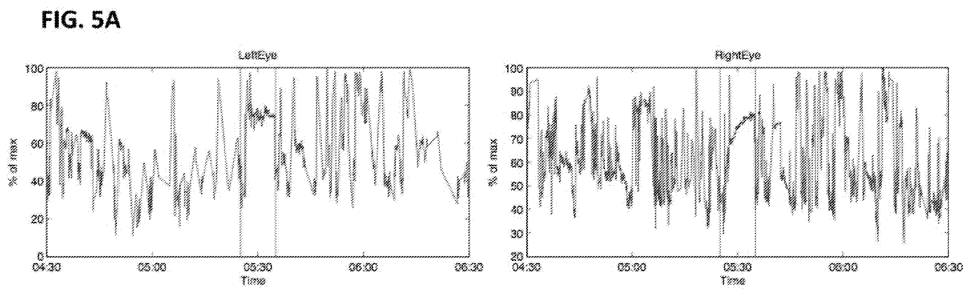

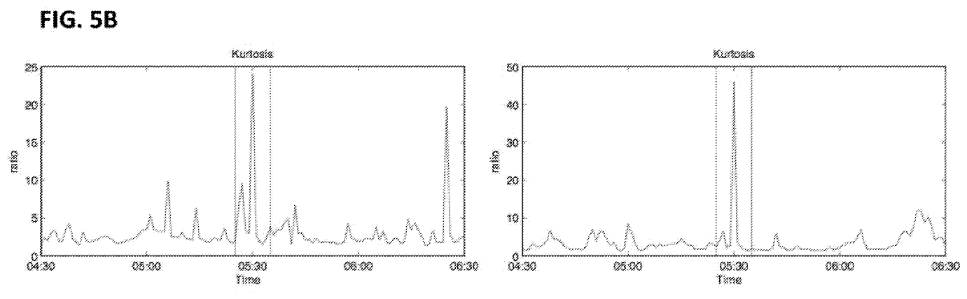

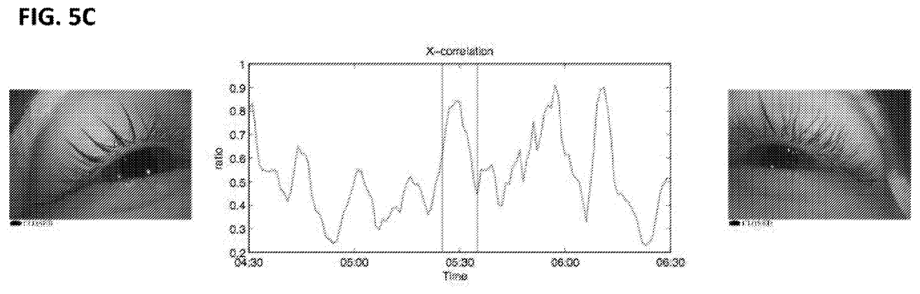

FIGS. 5A-5C depict the analysis of oculometric data derived from the same subject as in FIGS. 4A-4C experiencing a different epileptic event. FIG. 5A shows eye eccentricity as percent of max plotted against time for the left eye (FIG. 5A, left) and right eye (FIG. 5A, right). The Eye-Com Biosensor™ identified another epileptic event occurring at the 5:25 time stamp. The data was confirmed using the data from an EEG. The seizure lasted for about five seconds at a 2.5-3 Hz frequency.

FIG. 5B shows kurtosis over time in the left eye (FIG. 5B, left) and right eye (FIG. 5B, right). The data shows a spike in kurtosis by more than 5-fold and an increase in correlation of the eye movements between the right and left eye during and after the seizure. FIG. 5C shows the cross-correlation of eccentricity between the left eye and the right eye (FIG. 5C, center). The cross-correlation is similarly increased during and after the seizure. Photographs of the left eye (FIG. 5C, left) and right eye (FIG. 5C, right) during the epileptic event are also provided. The vertical red bars on the graphs denote the occurrence of an epileptic event.

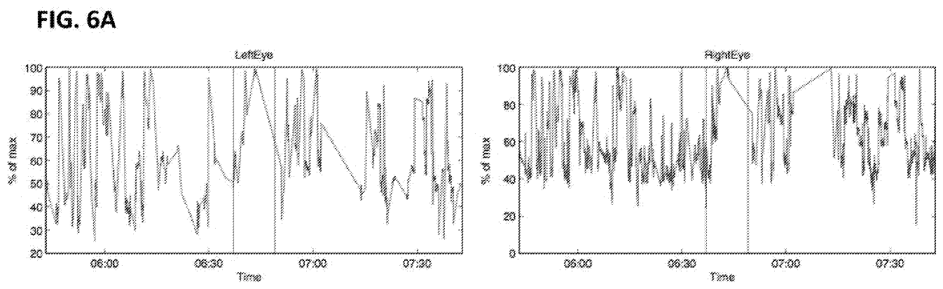

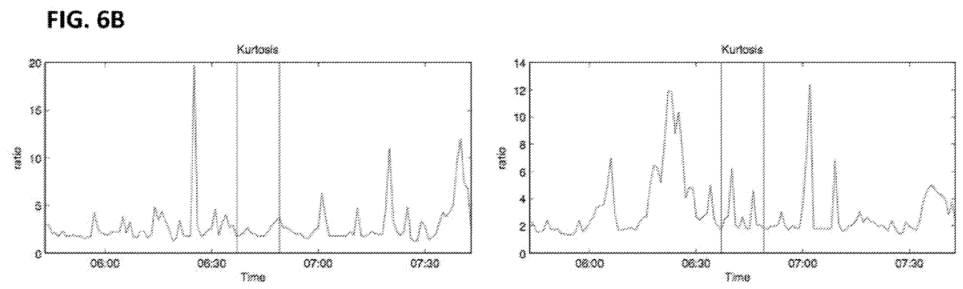

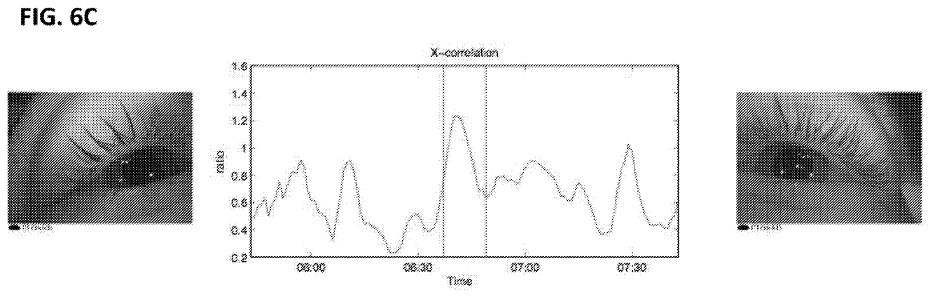

FIGS. 6A-6C depict the analysis of oculometric data derived from the same subject as in FIGS. 4A-4C and 5A-5C experiencing a different epileptic event. FIG. 6A shows eye eccentricity as percent of max plotted against time for the left eye (FIG. 6A, left) and right eye (FIG. 6A, right). The Eye-Com Biosensor™ identified another epileptic event occurring at the 6:37 time stamp. The data was confirmed using the data from an EEG; however, the event captured appears to include a loss of tracking, inferred from the continuous straight lines in the data. The seizure lasted for about five seconds at a 2.5 Hz frequency.

FIG. 6B shows kurtosis over time in the left eye (FIG. 6B, left) and right eye (FIG. 6B, right). FIG. 6C shows the cross-correlation of eccentricity between the left eye and the right eye (FIG. 6C, center). The processor unit has to interpolate to fill in missing data points. As such, the significant correlation seen here should be considered spurious. Photographs of the left eye (FIG. 6C, left) and right eye (FIG. 6C, right) during the epileptic event are also provided. The vertical red bars on the graphs denote the occurrence of an epileptic event.

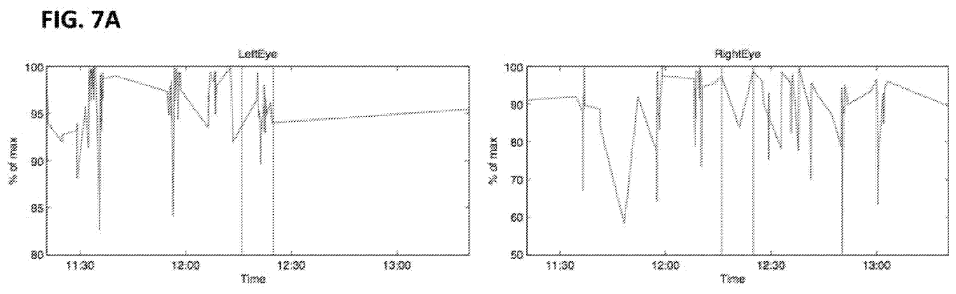

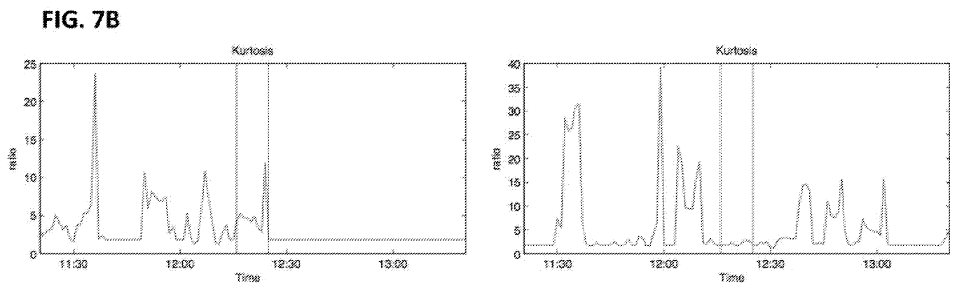

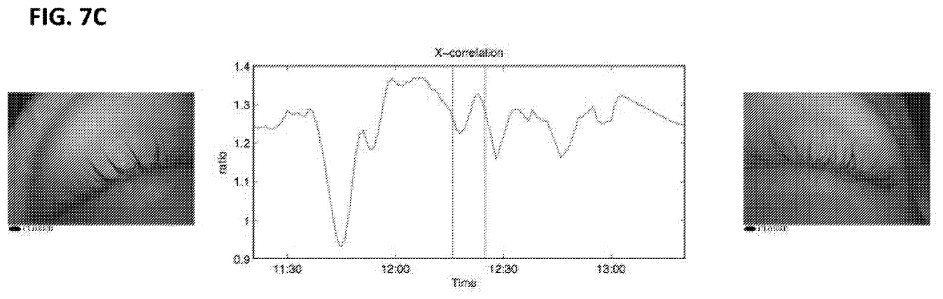

FIGS. 7A-7C depict the analysis of oculometric data derived from the same subject as in FIGS. 4A-4C, 5A-5C, and 6A-6C having closed eyes during a different epileptic event. FIG. 7A shows eye eccentricity as percent of max plotted against time for the left eye (FIG. 7A, left) and right eye (FIG. 7A, right). The Eye-Com Biosensor™ identified another epileptic event occurring at the 12:16 time stamp; however, the eyes appear to be closed for most of the event. The seizure was recorded at a 3 Hz frequency.

FIG. 7B shows kurtosis over time in the left eye (FIG. 7B, left) and right eye (FIG. 7B, right). FIG. 7C shows the cross-correlation of eccentricity between the left eye and the right eye (FIG. 7C, center). Photographs of the closed left eye (FIG. 7C, left) and right eye (FIG. 7C, right) during the epileptic event are also provided. The vertical red bars on the graphs denote the occurrence of an epileptic event. Because the eyes are closed, the oculometric data collected is not available for processing.

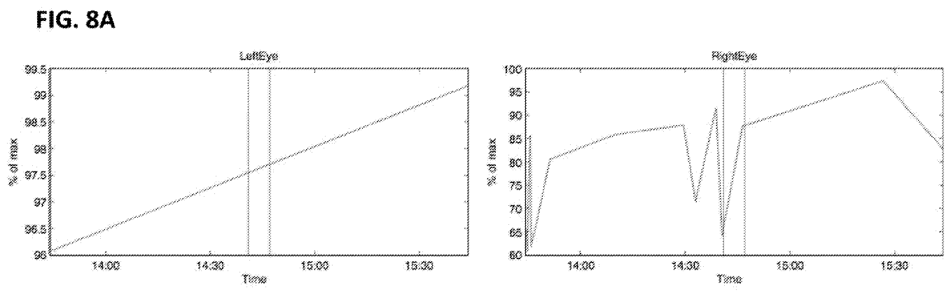

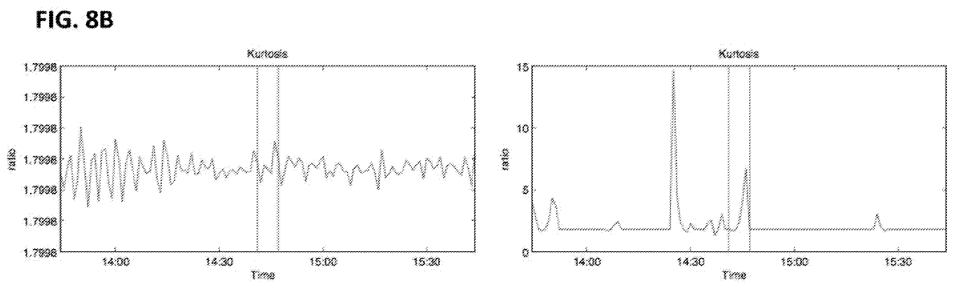

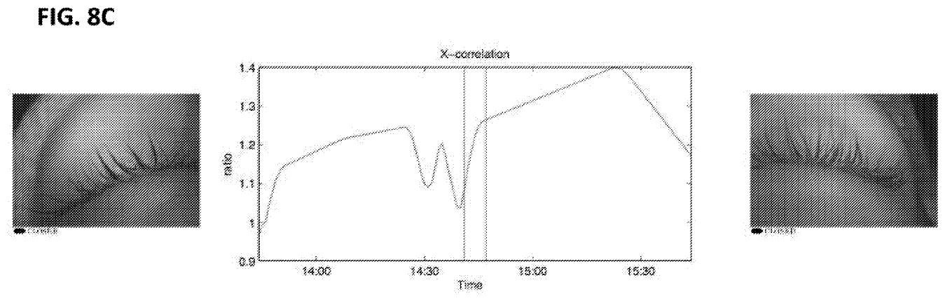

FIGS. 8A-8C depict the analysis of oculometric data derived from the same subject as in FIGS. 4A-4C, 5A-5C, 6A-6C, and 7A-7C having closed eyes during a different epileptic event. FIG. 8A shows eye eccentricity as percent of max plotted against time for the left eye (FIG. 8A, left) and right eye (FIG. 8A, right). The Eye-Com Biosensor™ identified another epileptic event occurring at the 14:41 time stamp; however, the eyes appear to be closed again for most of the event. The seizure was recorded at a 3 Hz frequency. The epileptic event recorded is a subclinical seizure onset.

FIG. 8B shows kurtosis over time in the left eye (FIG. 8B, left) and right eye (FIG. 8B, right). FIG. 8C shows the cross-correlation of eccentricity between the left eye and the right eye (FIG. 8C, center). Photographs of the closed left eye (FIG. 8C, left) and right eye (FIG. 8C, right) during the epileptic event are also provided. The vertical red bars on the graphs denote the occurrence of an epileptic event. As is the case in FIGS. 7A-7C, the eyes of the subject are closed, thus resulting in the oculometric data not being available for processing.

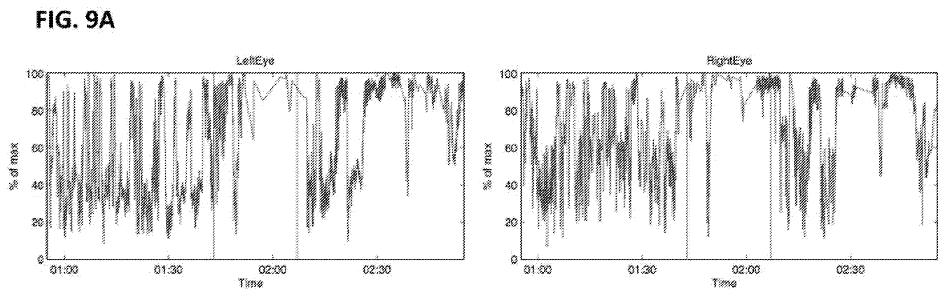

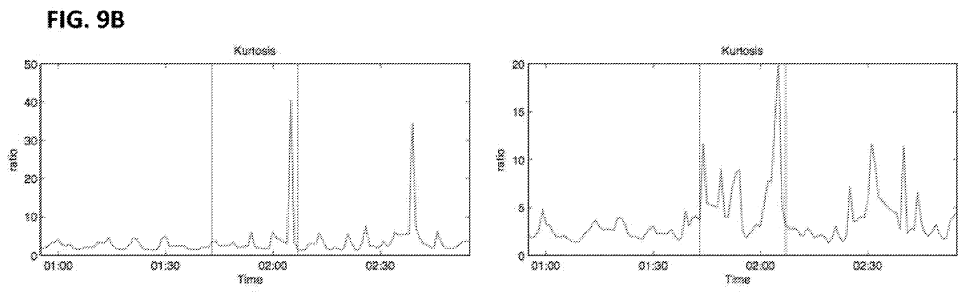

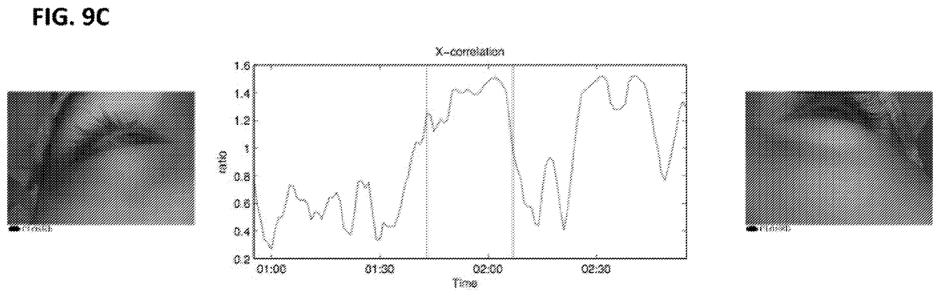

FIGS. 9A-9C depict the analysis of oculometric data derived from a subject experiencing two epileptic events within 30 seconds of each other and focuses specifically on the first epileptic event experienced. FIG. 9A shows eye eccentricity as percent of max plotted against time for the left eye (FIG. 9A, left) and right eye (FIG. 9A, right). The Eye-Com Biosensor™ identified another epileptic event occurring at the 1:43 time stamp. The data was confirmed using the data from an EEG. The seizure lasted for about twenty seconds at a 2.5-3 Hz frequency.

FIG. 9B shows kurtosis over time in the left eye (FIG. 9B, left) and right eye (FIG. 9B, right). There is a clear kurtosis spike in the left eye, but is less apparent in the right eye. FIG. 9C shows the cross-correlation of eccentricity between the left eye and the right eye (FIG. 9C, center). There is a significant synchronization of eye movements between the left and right eyes during and after the seizure. Photographs of the left eye (FIG. 9C, left) and right eye (FIG. 9C, right) during the epileptic event are also provided. The vertical red bars on the graphs denote the occurrence of an epileptic event.

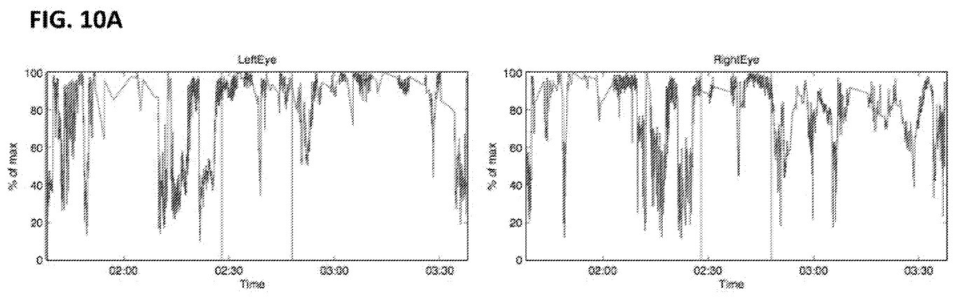

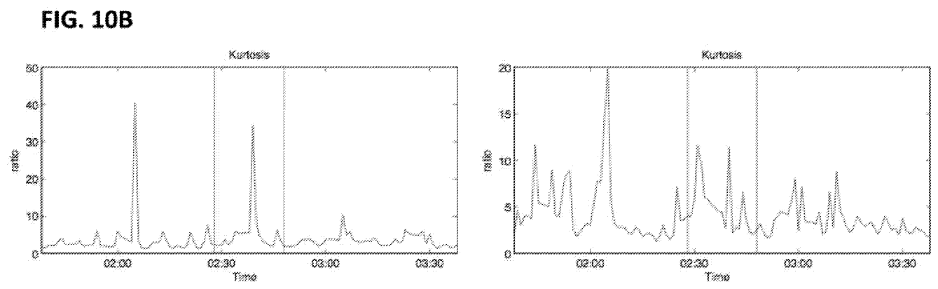

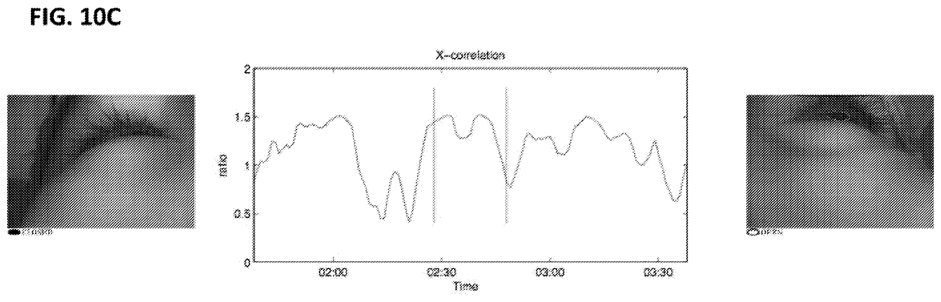

FIGS. 10A-10C depict the analysis of oculometric data derived from the same subject as in FIGS. 9A-9C focusing specifically on the second epileptic event experienced in a 30 second-window. FIG. 10A shows eye eccentricity as percent of max plotted against time for the left eye (FIG. 10A, left) and right eye (FIG. 10A, right). The Eye-Com™ identified another epileptic event occurring at the 2:28 time stamp. The data was confirmed using the data from an EEG. The seizure lasted for about twenty seconds at a 3 Hz frequency.

FIG. 10B shows kurtosis over time in the left eye (FIG. 10B, left) and right eye (FIG. 10B, right). There is a significant increase in kurtosis, signaling an increased stability of the eye movements and decreased movements during the time of the seizure. FIG. 10C shows the cross-correlation of eccentricity between the left eye and the right eye (FIG. 10C, center). Photographs of the left eye (FIG. 10C, left) and right eye (FIG. 10C, right) during the epileptic event are also provided. The vertical red bars on the graphs denote the occurrence of an epileptic event. The continuous oculometric data was analyzed in relation to the seizure on and offset which was identified by the gold standard, EEG, which was performed concurrently.

Of the oculometric data captured, eccentricity, a calculated variable, which is a function of the visible x width and y width of the pupil, was the most sensitive and specific indicator of seizures under the tested conditions. The eccentricity in these measurements was a combined variable which included the occlusion of the pupil relative to the eyelid position and sides of the eye, pupil area and blink frequency. The stability of the eyes may be inferred from the observed eccentricity of the pupils, marked by the kurtosis of a 5-second moving window.

The captured data illustrated that kurtosis, or the change in the probability distribution of eccentricity for each eye over a 5-second running window, correlated with seizure. The kurtosis analyzed how stable a parameter, in this case in the eye movement, appeared. The standard kurtosis measurements of eye eccentricity taken at a 5-second moving window were most sensitive for an absence seizure, which on average lasted between 10-15 seconds. The larger the kurtosis, the less variable the distribution or the less the eye was moving. The smaller the kurtosis, the more the eye was moving. The kurtosis of other eye movements was also measured.

Another indicator of seizure was an increase in in-sync behavior of the eyes during and after a seizure. Kurtosis of eye eccentricity for both eyes was cross-correlated with the in-sync behavior of both eyes during and after the seizure. All analyses were performed with MATLAB.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### REFERENCES

1. Afifi A K, Corbett J J, Thompson H S, Wells K K. Seizure-induced miosis and ptosis: association with temporal lobe magnetic resonance imaging abnormalities. J Child Neurol. 1990 April; 5(2):142-6.
2. Afraimovich V S, Verichev N N, Rabinovich M I (1986). Stochastic synchronization of oscillation in dissipative systems. Radiophys. Quantum Electron 29:795-803.
3. Akman C L, Montenegro M A, Jacob S, Eck K, Chiraboga C, Gilliam F. Seizure frequency in children with epilepsy: factors influencing accuracy and parental awareness. Seizure 2009 September; 18(7):524-9.
4. Arzimanoglou A, Guerrini R, Aicardi J. Aicardi's Epilepsy in Children. Lipincott Williams and Wilkins, 2004.
5. Bauder F, Wohlrab G, Schmitt B. Neonatal Seizures: Eyes Open or Closed. Epilepsia 2007 48(2):394-396
6. Centeno et al. Epilepsy causing pupillary hippus: an unusual semiology. Epilepsia, 52(8):e93-e96, 2011.
7. Chua et al. Application of higher order statistics/spectra in biomedical signals—A review. Medical Eng. Phys. 2010. 32: 679-689.
8. Chung S, Gerber P, Kirlin K A. Ictal Eye Closure is a Reliable Indicator for psychogenic nonepileptic seizures. Neurology 2006; 66:1730-1731
9. DeToledo et al., Patterns of involvement of facial muscles during epileptic and nonepileptic events: Review of 654 events. Neurology 1996; 47: 621-625.
10. Devinsky O. Effects of Seizures on Autonomic and Cardiovascular Function. Epilepsy Curr. 2004 March; 4(2):43-46.
11. Fujisaka H, Yamada T (1983). Stability Theory of Synchronized Motion in Coupled-Oscillator Systems. Progr Theoret Phys 69:32-47.
12. Hoppe C, Annkathrin P, Christian E. Accuracy of Patient Seizure Counts. Arch Neurol. 2007; 64(11):1595-1599.
13. Korff C M, Nordli D R. Paroxysmal Events in Infants: Persistent Eye Closure Makes Seizures Unlikely. Pediatrics 2005; 116:e485-e486.
14. Lee J, Devinsky O. The role of autonomic dysfunction in sudden unexplained death in epilepsy patients. Rev Neurol Dis. 2005 Spring; 2(2):61-9.
15. Malone A, Ryan C A, Fitzgerald A, Burgoyne L, Connolly S, Boylan G B. Interobserver agreement in neonatal seizure identification. Epilepsia. 2009 September; 50(9):2097-101. Epub 2009 Jun. 1.
16. Mejer R, Dittrich H, Schulze-Bonhage A, Aertsen A. Detecting epileptic seizures in long-term human EEG: a new approach to automatic online and real-time detection and classification of polymorphic seizure patterns. J Clin Neurophysiol. 2008 June; 25(3):119-31.
17. Mendel J. M. Tutorial on Higher-Order Statistics (Spectra) in Signal Processing and System Theory: Theoretical Results and Some Applications. (1991). IEEE, 79(3): 278-305.
18. Orren M M, Mirsky A F. Relation between Ocular Manifestations and Onset of Spike and Wave Discharges in Petit Mal Epilepsy. Epilpesia 1975; 16:771-779
19. Rafal R, Laxer K D, Janowsky J S. Seizures triggered by blinking in a non-photosensitive epileptic. J. Neurology 1986; 49: 445-447.
20. Reconstructing Speech from Human Auditory Cortex Pasley B N, David S V, Mesgarani N, Flinker A, Shamma S A, et al. (2012). PLoS Biol 10(1): e1001251.
21. Rosenblum M G, Pikovsky A S, Kurths J (1996). Phase Synchronization of Chaotic Oscillators. Phys Rev Lett 76:1804-1807.
22. Scaramelli A, Braga P, Avellanal A, Bogacz A, Camejo C, Rega I, Messano T, and Arciere B. (2009). Seizure. 18(4): 246-250.
23. Toledo J C, Ramsay R E. Patterns of involvement of facial muscles during epileptic and non-epileptic events: Review of 654 events. Neurology 1996; 47(3): 621-625
24. Tomson T, Nashef L, Ryvlin P. Sudden unexpected death in epilepsy: current knowledge and future directions. Lancet Neurol. 2008 November; 7(11):1021-31. Epub 2008 Sep. 19.
25. Torch, W, “Eye-Com Oculometric, EEG and Cognitive Performance Monitoring of Sleep-Deprived Subjects in Drive/Flight Simulator Outfitted with Tele-Communications Transmission, Recording and Alerting Technology, “DoD HFE TAG, SusOps Conference, Aladdin Hotel, Las Vegas, Nev., May 17, 2006.
26. Torch, W., Russo, M. Cardillo, C and Thomas, M. “Eye-Com Biosensor, Communicator and Controller Systems for Assessing Cognitive State via Oculomoter Signals”, in Foundations of Augmented Cognition, 4^{th} edition, edited by D.

Schmorrow et al, 2007, Strategic Analysis, Inc. and Augmented Cognition International (ACI), pp 258-265.
27. Torch, W., Russo, M. Cardillo, C and Thomas, M. “Eye-Com Biosensor, Communicator and Controller Systems for Assessing Cognitive State via Oculomotor Signals, presented at the 4^{th} International Augmented Cognition Conference and the 51^{st} Annual Meeting of the Human Factors and Ergonomics Society, Baltimore, Md., Oct. 1-5, 2007.
28. Torch, W., Soldier Mounted Eye-Tracking & Control Systems: Eye-Com Biosensor, Communicator and Controller; presented at the Telemedicine and Advanced Technology Research Center (TATRC/MRMC), Ocular Health Product Line Review, Ft. Detrick, Md., Jul. 10, 2007.
29. Torch, W. and Cardillo, C. Eye-Com Technology for Measuring Alertness and Design of a Statistically Derived, Composite Oculometric Fatigue Index (COFI). Jan. 11-15, 2009. Washington, D.C. Transportation Research Board—88^{th} Annual Meeting (Abstract accepted for Presentation).
30. Torch, W. and Cardillo, C. Validation of the PERCLOS Loss of Alertness and other Oculometric Measures as an Index of Clinical Causes of Driver Impairment. Mar. 24-26, 2009. Boston, Mass., Fatigue Management in Transportation Operations —International Conference (Abstract accepted for Presentation).
31. Torch, W. The Eye-Com Biosensor Communicator & Controller as a New Assistive Communications and Controller Technology for the Disabled; March 2009, International Conference on New Technologies and Disabilities, California State University-Northridge, Los Angeles, Calif., Mar. 18, 2009.
32. Torch, W. and Cardillo, C. Oculometric Measures as an Index of Driver Impairment. Apr. 26-29, 2009. The Hague, Netherlands. 21^{st} World Congress of International Traffic Medicine Association.
33. Torch, W., Cardillo C., Russo, M., Publicover, N., Gutierrez, E., McMullen Jr., S., Parseghian, Z., and Martin, M., Oculometric Indices Associated with Drowsiness and Performance Vigilance Impairment in Sleep Deprived Normal, Sleep Apnea, Narcolepsy and ADD/ADHD Subjects. 23^{rd} Annual Meeting of the Associated Professional Sleep Society, Seattle, Wash., Jun. 6-11, 2009 (Abstract submitted for Presentation).
34. Volpe J J. (2001) Neonatal Seizures. In Volpe J J (Ed) Neurology of the Newborn. WB Saunders, Philadelphia, pp 178-214.
35. Watemberg N Tziperman B, Dabby R, Hasa M, Zehavi L, Lerman-Sagie T. Adding Video Recording Increases the Diagnostic Yield of Routine EEGs in children with frequent paroxysmal events. Epilepsia 2005 46 (5):716-719

What is claimed is:

1. A method of detecting and/or predicting an epileptic event in a subject, the method comprising:
a) measuring a change in one or more oculometric parameters of at least one eye of the subject overtime using a measuring device to obtain oculometric data from the subject;
b) performing a first order statistical analysis of the oculometric data;
c) determining the presence or absence of a change relative to baseline in the first order statistical analysis of the oculometric data; and
d) indicating that an epileptic event has been detected and/or predicted when the determining indicates the presence or absence of a change in the first order statistical analysis relative to baseline.

2. The method of claim 1, wherein the one or more oculometric parameters comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.

3. The method of claim 1 or 2, wherein the measuring comprises measuring a change in two or more of the oculometric parameters.

4. The method of any one of claims 1-3, wherein eye eccentricity is a function of visible x-width and y-width of the pupil of an eye.

5. The method of claim 4, wherein eye eccentricity changes as the eyelid position, position of the sides of the eye, pupil area, and/or blink frequency change(s).

6. The method of any one of claims 1-5, wherein the first order statistical analysis of the oculometric data comprises multiple regression analysis and/or mean calculations of the oculometric data.

7. The method of any one of claims 1-6, wherein the measuring device is configured to obtain oculometric data from the subject for about thirty minutes.

8. The method of any one of claims 1-6, wherein the measuring device is configured to obtain oculometric data from the subject for about fifteen minutes.

9. The method of claim 7 or 8, wherein the performing the first order statistical analysis of the oculometric data occurs in a ten-second running window.

10. The method of claim 7 or 8, wherein the performing the first order statistical analysis of the oculometric data occurs in a five-second running window.

11. The method of any one of claims 1-10, wherein the measuring device is an eye tracking device.

12. The method of claim 11, wherein the eye tracking device comprises one or more cameras.

13. The method of claim 12, wherein the eye tracking device further comprises a video recorder and/or a sensor.

14. The method of claim 13, wherein the eye tracking device is a wearable device configured to be worn on the head of the subject.

15. The method of claim 14, wherein the one or more cameras of the wearable device is located at a distance of one or more centimeters from the eyes of the subject.

16. The method of any one of claims 1-13, wherein the eye tracking device is a contact lens.

17. The method of any one of claims 1-16, wherein the performing the first order statistical analysis of the oculometric data comprises performing multiple regression analysis of the oculometric data.

18. The method of claim 17, wherein the determining the presence or absence of a change in the first order statistical analysis of the oculometric data comprises determining the presence or absence of an increased correlation of one or more oculometric parameters with the epileptic event.

19. The method of claim 18, wherein the determining the presence or absence of an increased correlation of one or more oculometric parameters with the epileptic event comprises determining the presence or absence of an increased correlation of eye eccentricity with the epileptic event.

20. The method of any one of claims 1-19, wherein the oculometric data from the subject is captured at about 30 frames per second or more.

21. The method of any one of claims 1-19, wherein the oculometric data from the subject is captured at about 60 frames per second or more.

22. The method of any one of claims 1-19, wherein the oculometric data from the subject is captured at about 100 frames per second or more.

23. The method of any one of claims 1-19, wherein the oculometric data from the subject is captured at about 200 frames per second or more.

24. The method of any one of claims 1-23, further comprising measuring a change in one or more facial biometrics of the subject to provide facial biometrics data.

25. The method of claim 24, further comprising performing a first order statistical analysis of the facial biometrics data.

26. The method of claim 25, further comprising determining the presence or absence of a change relative to baseline in the first order statistical analysis of the facial biometrics data.

27. The method of any one of claims 24-26, wherein the one or more facial biometrics comprises distance between the eyes; distance between the eyelids; width of the nose; center of the nose; depth of the eye sockets; shape of the cheekbones; length of the jawline; distance between the mouth edges; center of the mouth; and/or focal weakness.

28. The method of any one of claims 1-27, further comprising measuring prodromal changes of the oculometric data and/or facial biometrics data.

29. The method of claim 28, wherein the prodromal changes occur one or more days before the epileptic event.

30. The method of claim 28, wherein the prodromal changes occur one or more hours before the epileptic event.

31. The method of claim 28, wherein the prodromal changes occur one or more seconds before the epileptic event.

32. The method of any one of claims 28-31, further comprising performing a first order statistical analysis of the prodromal changes of the oculometric data and/or facial biometrics data.

33. The method of claim 32, further comprising determining the presence or absence of a change relative to baseline in the first order statistical analysis of the prodromal changes of the oculometric data and/or facial biometrics data.

34. The method of any one of claims 1-33, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.

35. The method of any one of claims 1-34, wherein the indicating that the epileptic event has been detected and/or predicted comprises providing an alert to the subject or a caregiver of the subject.

36. The method of any one of claims 1-35, further comprising providing a responsive neurostimulation to the subject, wherein the responsive neurostimulation is sufficient to reduce the effect of the epileptic event, when the epileptic event is detected and/or predicted.

37. The method of any one of claims 1-36, further comprising transmitting an electric current through the neck of the subject for which an epileptic event has been detected and/or predicted to a vagus nerve in the subject for which an epileptic event has been detected and/or predicted, wherein the electric current is sufficient to terminate the epileptic event, when the epileptic event is detected and/or predicted.

38. The method of any one of claims 1-37, further comprising administering an effective amount of an anti-epileptic drug to the subject, when the epileptic event is detected and/or predicted.

39. The method of claim 38, wherein the anti-epileptic drug comprises one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.

40. The method of any one of claims 1-39, wherein measuring a change in one or more oculometric parameters of at least one eye comprises measuring a change in one or more oculometric parameters of both the left eye and the right eye.

41. The method of claim 40, wherein the one or more oculometric parameters comprise left and right eye movements.

42. The method of claim 40 or 41, further comprising cross-correlating oculometric data of a left eye and oculometric data of a right eye of the subject.

43. The method of any one of claims 40-42, wherein the determining the presence or absence of the change relative to baseline in the first order statistical analysis of the oculometric data comprises determining the presence of an increase in the synchronization of eye movements between the left eye and the right eye of the subject relative to baseline.

44. The method of any one of claims 1-43, further comprising cross-correlating the first order statistical analysis of the oculometric data.

45. The method of any one of claims 1-43, further comprising cross-correlating the first order statistical analysis of the facial biometrics data.

46. The method of any one of claims 1-45, further comprising performing a second order statistical analysis of the oculometric data and/or facial biometrics data.

47. The method of any one of claims 1-46, further comprising performing a higher order statistical analysis of the oculometric data and/or facial biometrics data.

48. The method of claim 47, wherein the higher order statistical analysis of the oculometric data and/or facial biometrics data comprises kurtosis.

49. The method of claim 48, further comprising determining the presence or absence of a change relative to baseline in the higher order statistical analysis of the oculometric data and/or facial biometrics data.

50. The method of claim 49, wherein the determining the presence or absence of a change relative to baseline in the higher order statistical analysis of the oculometric data and/or facial biometrics data comprises determining the presence of a change from frequency independence to inter-frequency dependence of the oculometric data and/or facial biometrics data.

51. The method of claim 49, wherein the determining the presence or absence of a change relative to baseline in the higher order statistical analysis of the oculometric data and/or facial biometrics data comprises determining change in synchronization of the oculometric data and/or facial biometrics data.

52. The method of claim 51, wherein the determining synchronization of the oculometric data and/or facial biometrics data comprises determining frequency synchronization of the oculometric data and/or facial biometrics data.

53. The method of claim 52, wherein the determining frequency synchronization comprises determining synchronization of dependent and/or uncoupled frequencies of the oculometric data and/or facial biometrics data.

54. The method of claim 49, wherein the determining the presence or absence of a change in the first order statistical analysis of the oculometric data and/or facial biometrics data comprises determining the presence of positive excess kurtosis of the oculometric data and/or facial biometrics data.

55. The method of claim 54, wherein the determining the presence of positive excess kurtosis of the oculometric data comprises determining the presence of positive excess kurtosis of eye eccentricity.

56. The method of claim 55, wherein the positive excess kurtosis is 10 or more.

57. The method of claim 55, wherein the positive excess kurtosis is 15 or more.

58. The method of any one of claims 1-57, wherein the determining step utilizes machine learning.

59. The method of any one of claims 1-58, further comprising cross-correlating the higher order statistical analysis of the oculometric data.

60. The method of any one of claims 1-58, further comprising cross-correlating the higher order statistical analysis of the facial biometrics data.

61. The method of any one of claims 1-60, further comprising measuring at least one electroencephalogram signal of the subject.

62. The method of claim 61, further comprising confirming the presence or absence of a change relative to baseline in the first order statistical analysis of the oculometric data using the at least one electroencephalogram signal.

63. The method of any one of claims 1-62, wherein the epileptic event in the subject is detected and/or predicted in the absence of measuring an electroencephalogram signal of the subject.

64. A method of identifying and treating epilepsy in a subject, the method comprising:
a) measuring a change in one or more oculometric parameters of at least one eye of the subject overtime using a measuring device to obtain oculometric data from the subject;
b) performing a first order statistical analysis of the oculometric data;
c) determining the presence or absence of a change relative to baseline in the first order statistical analysis of the oculometric data;
d) identifying the subject as having an epileptic event and/or as at risk of an epileptic event when the determining indicates the presence or absence of a change in the first order statistical analysis of the oculometric data relative to baseline; and
e) administering an effective amount of an anti-epileptic drug to the subject identified as having an epileptic event and/or as at risk of an epileptic event.

65. The method of claim 64, wherein the one or more oculometric parameters comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.

66. The method of claim 64 or 65, wherein the measuring comprises measuring a change in two or more of the oculometric parameters.

67. The method of any one of claims 64-66, wherein eye eccentricity is a function of visible x-width and y-width of the pupil of an eye.

68. The method of claim 67, wherein eye eccentricity changes as the eyelid position, position of the sides of the eye, pupil area, and/or blink frequency change(s).

69. The method of any one of claims 64-68, wherein the first order statistical analysis of the oculometric data comprises multiple regression analysis and/or mean calculations of the oculometric data.

70. The method of any one of claims 64-69, wherein the measuring device is configured to obtain oculometric data from the subject for about thirty minutes.

71. The method of any one of claims 64-69, wherein the measuring device is configured to obtain oculometric data from the subject for about fifteen minutes.

72. The method of claim 70 or 71, wherein the performing the first order statistical analysis of the oculometric data occurs in a ten-second running window.

73. The method of claim 70 or 71, wherein the performing the first order statistical analysis of the oculometric data occurs in a five-second running window.

74. The method of any one of claims 64-74, wherein the measuring device is an eye tracking device.

75. The method of claim 74, wherein the eye tracking device comprises one or more cameras.

76. The method of claim 75, wherein the eye tracking device further comprises a video recorder and/or a sensor.

77. The method of claim 76, wherein the eye tracking device is a wearable device configured to be worn on the head of the subject.

78. The method of claim 77, wherein the one or more cameras of the wearable device is located at a distance of one or more centimeters from the eyes of the subject.

79. The method of any one of claims 64-76, wherein the eye tracking device is a contact lens.

80. The method of any one of claims 64-79, wherein the performing the first order statistical analysis of the oculometric data comprises performing multiple regression analysis of the oculometric data.

81. The method of claim 80, wherein the determining the presence or absence of a change in the first order statistical analysis of the oculometric data comprises determining the presence or absence of an increased correlation of one or more oculometric parameters with the epileptic event.

82. The method of claim 81, wherein the determining the presence or absence of an increased correlation of one or more oculometric parameters with the epileptic event comprises determining the presence or absence of an increased correlation of eye eccentricity with the epileptic event.

83. The method of any one of claims 64-82, wherein the oculometric data from the subject is captured at about 30 frames per second or more.

84. The method of any one of claims 64-82, wherein the oculometric data from the subject is captured at about 60 frames per second or more.

85. The method of any one of claims 64-82, wherein the oculometric data from the subject is captured at about 100 frames per second or more.

86. The method of any one of claims 64-82, wherein the oculometric data from the subject is captured at about 200 frames per second or more.

87. The method of any one of claims 64-86, further comprising measuring a change in one or more facial biometrics of the subject to provide facial biometrics data.

88. The method of claim 87, further comprising performing a first order statistical analysis of the facial biometrics data.

89. The method of claim 89, further comprising determining the presence or absence of a change relative to baseline in the first order statistical analysis of the facial biometrics data.

90. The method of any one of claims 87-89, wherein the one or more facial biometrics comprises distance between the eyes; distance between the eyelids; width of the nose; center of the nose; depth of the eye sockets; shape of the cheekbones; length of the jawline; distance between the mouth edges; center of the mouth; and/or focal weakness.

91. The method of any one of claims 64-90, further comprising measuring prodromal changes of the oculometric data and/or facial biometrics data.

92. The method of claim 91, wherein the prodromal changes occur one or more days before the epileptic event.

93. The method of claim 91, wherein the prodromal changes occur one or more hours before the epileptic event.

94. The method of claim 91, wherein the prodromal changes occur one or more seconds before the epileptic event.

95. The method of any one of claims 91-94, further comprising performing a first order statistical analysis of the prodromal changes of the oculometric data and/or facial biometrics data.

96. The method of claim 95, further comprising determining the presence or absence of a change relative to baseline in the first order statistical analysis of the prodromal changes of the oculometric data and/or facial biometrics data.

97. The method of any one of claims 64-96, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.

98. The method of any one of claims 64-97, wherein the identifying comprises providing an alert to the subject or a caregiver of the subject.

99. The method of any one of claims 64-98, further comprising providing a responsive neurostimulation to the subject, wherein the responsive neurostimulation is sufficient to reduce the effect of the epileptic event, when the subject is identified as having an epileptic event and/or as at risk of an epileptic event.

100. The method of any one of claims 64-99, further comprising transmitting an electric current through the neck of the subject for which an epileptic event has been detected and/or predicted to a vagus nerve in the subject for which an epileptic event has been detected and/or predicted, wherein the electric current is sufficient to terminate the epileptic event, when the subject is identified as having an epileptic event and/or as at risk of an epileptic event.

101. The method of claim 64, wherein the anti-epileptic drug comprises one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.

102. The method of any one of claims 64-101, wherein measuring a change in one or more oculometric parameters of at least one eye comprises measuring a change in one or more oculometric parameters of both the left eye and the right eye.

103. The method of claim 102, wherein the one or more oculometric parameters comprise left and right eye movements.

104. The method of claim 102 or 103, further comprising cross-correlating oculometric data of a left eye and oculometric data of a right eye of the subject.

105. The method of any one of claims 102-104, wherein the determining the presence or absence of the change relative to baseline in the first order statistical analysis of the oculometric data comprises determining the presence of an increase in the synchronization of eye movements between the left eye and the right eye of the subject relative to baseline.

106. The method of any one of claims 64-105, further comprising cross-correlating the first order statistical analysis of the oculometric data.

107. The method of any one of claims 64-105, further comprising cross-correlating the first order statistical analysis of the facial biometrics data.

108. The method of any one of claims 64-107, further comprising performing a second order statistical analysis of the oculometric data and/or facial biometrics data.

109. The method of any one of claims 64-108, further comprising performing a higher order statistical analysis of the oculometric data and/or facial biometrics data.

110. The method of claim 109, wherein the higher order statistical analysis of the oculometric data and/or facial biometrics data comprises kurtosis.

111. The method of claim 110, further comprising determining the presence or absence of a change relative to baseline in the higher order statistical analysis of the oculometric data and/or facial biometrics data.

112. The method of claim 111, wherein the determining the presence or absence of a change relative to baseline in the higher order statistical analysis of the oculometric data and/or facial biometrics data comprises determining the presence of a change from frequency independence to inter-frequency dependence of the oculometric data and/or facial biometrics data.

113. The method of claim 111, wherein the determining the presence or absence of a change relative to baseline in the higher order statistical analysis of the oculometric data and/or facial biometrics data comprises determining change in synchronization of the oculometric data and/or facial biometrics data.

114. The method of claim 113, wherein the determining synchronization of the oculometric data and/or facial biometrics data comprises determining frequency synchronization of the oculometric data and/or facial biometrics data.

115. The method of claim 114, wherein the determining frequency synchronization comprises determining synchronization of dependent and/or uncoupled frequencies of the oculometric data and/or facial biometrics data.

116. The method of claim 111, wherein the determining the presence or absence of a change in the first order statistical analysis of the oculometric data and/or facial biometrics data comprises determining the presence of positive excess kurtosis of the oculometric data and/or facial biometrics data.

117. The method of claim 116, wherein the determining the presence of positive excess kurtosis of the oculometric data comprises determining the presence of positive excess kurtosis of eye eccentricity.

118. The method of claim 117, wherein the positive excess kurtosis is 10 or more.

119. The method of claim 117, wherein the positive excess kurtosis is 15 or more.

120. The method of any one of claims 64-119, wherein the determining step utilizes machine learning.

121. The method of any one of claims 64-120, further comprising cross-correlating the higher order statistical analysis of the oculometric data.

122. The method of any one of claims 64-120, further comprising cross-correlating the higher order statistical analysis of the facial biometrics data.

123. The method of any one of claims 64-120, further comprising measuring at least one electroencephalogram signal of the subject.

124. The method of claim 123, further comprising confirming the presence or absence of a change relative to baseline in the first order statistical analysis of the oculometric data using the at least one electroencephalogram signal.

125. The method of any one of claims 64-124, wherein the epileptic event in the subject is detected and/or predicted in the absence of measuring an electroencephalogram signal of the subject.

126. A method of identifying and treating epilepsy in a subject, the method comprising:
a) measuring a change in one or more oculometric parameters of at least one eye of the subject overtime using a measuring device to obtain oculometric data from the subject;
b) performing a first order statistical analysis of the oculometric data;
c) determining the presence or absence of a change relative to baseline in the first order statistical analysis of the oculometric data;
d) identifying the subject as having an epileptic event and/or as at risk of an epileptic event when the determining indicates the presence or absence of a change in the first order statistical analysis of the oculometric data relative to baseline; and
e) transmitting an electric current through the neck of the subject identified as having an epileptic event and/or as at risk of an epileptic event to a vagus nerve in the subject, wherein the electric current is sufficient to terminate the epileptic event.

127. The method of claim 126, wherein the one or more oculometric parameters comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.

128. The method of claim 126 or 127, wherein the measuring comprises measuring a change in two or more of the oculometric parameters.

129. The method of any one of claims 126-128, wherein eye eccentricity is a function of visible x-width and y-width of the pupil of an eye.

130. The method of claim 129, wherein eye eccentricity changes as the eyelid position, position of the sides of the eye, pupil area, and/or blink frequency change(s).

131. The method of any one of claims 126-130, wherein the first order statistical analysis of the oculometric data comprises multiple regression analysis and/or mean calculations of the oculometric data.

132. The method of any one of claims 126-131, wherein the measuring device is configured to obtain oculometric data from the subject for about thirty minutes.

133. The method of any one of claims 126-131, wherein the measuring device is configured to obtain oculometric data from the subject for about fifteen minutes.

134. The method of claim 132 or 133, wherein the performing the first order statistical analysis of the oculometric data occurs in a ten-second running window.

135. The method of claim 132 or 133, wherein the performing the first order statistical analysis of the oculometric data occurs in a five-second running window.

136. The method of any one of claims 126-135, wherein the measuring device is an eye tracking device.

137. The method of claim 136, wherein the eye tracking device comprises one or more cameras.

138. The method of claim 137, wherein the eye tracking device further comprises a video recorder and/or a sensor.

139. The method of claim 138, wherein the eye tracking device is a wearable device configured to be worn on the head of the subject.

140. The method of claim 139, wherein the one or more cameras of the wearable device is located at a distance of one or more centimeters from the eyes of the subject.

141. The method of any one of claims 126-138, wherein the eye tracking device is a contact lens.

142. The method of any one of claims 126-141, wherein the performing the first order statistical analysis of the oculometric data comprises performing multiple regression analysis of the oculometric data.

143. The method of claim 142, wherein the determining the presence or absence of a change in the first order statistical analysis of the oculometric data comprises determining the presence or absence of an increased correlation of one or more oculometric parameters with the epileptic event.

144. The method of claim 143, wherein the determining the presence or absence of an increased correlation of one or more oculometric parameters with the epileptic event comprises determining the presence or absence of an increased correlation of eye eccentricity with the epileptic event.

145. The method of any one of claims 126-144, wherein the oculometric data from the subject is captured at about 30 frames per second or more.

146. The method of any one of claims 126-144, wherein the oculometric data from the subject is captured at about 60 frames per second or more.

147. The method of any one of claims 126-144, wherein the oculometric data from the subject is captured at about 100 frames per second or more.

148. The method of any one of claims 126-144, wherein the oculometric data from the subject is captured at about 200 frames per second or more.

149. The method of any one of claims 126-148, further comprising measuring a change in one or more facial biometrics of the subject to provide facial biometrics data.

150. The method of claim 149, further comprising performing a first order statistical analysis of the facial biometrics data.

151. The method of claim 150, further comprising determining the presence or absence of a change relative to baseline in the first order statistical analysis of the facial biometrics data.

152. The method of any one of claims 149-151, wherein the one or more facial biometrics comprises distance between the eyes; distance between the eyelids; width of the nose; center of the nose; depth of the eye sockets; shape of the cheekbones; length of the jawline; distance between the mouth edges; center of the mouth; and/or focal weakness.

153. The method of any one of claims 126-152, further comprising measuring prodromal changes of the oculometric data and/or facial biometrics data.

154. The method of claim 153, wherein the prodromal changes occur one or more days before the epileptic event.

155. The method of claim 153, wherein the prodromal changes occur one or more hours before the epileptic event.

156. The method of claim 153, wherein the prodromal changes occur one or more seconds before the epileptic event.

157. The method of any one of claims 153-156, further comprising performing a first order statistical analysis of the prodromal changes of the oculometric data and/or facial biometrics data.

158. The method of claim 157, further comprising determining the presence or absence of a change relative to baseline in the first order statistical analysis of the prodromal changes of the oculometric data and/or facial biometrics data.

159. The method of any one of claims 126-158, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.

160. The method of any one of claims 126-159, wherein the identifying comprises providing an alert to the subject or a caregiver of the subject.

161. The method of any one of claims 126-160, wherein measuring a change in one or more oculometric parameters of at least one eye comprises measuring a change in one or more oculometric parameters of both the left eye and the right eye.

162. The method of claim 161, wherein the one or more oculometric parameters comprise left and right eye movements.

163. The method of claim 161 or 162, further comprising cross-correlating oculometric data of a left eye and oculometric data of a right eye of the subject.

164. The method of any one of claims 161-163, wherein the determining the presence or absence of the change relative to baseline in the first order statistical analysis of the oculometric data comprises determining the presence of an increase in the synchronization of eye movements between the left eye and the right eye of the subject relative to baseline.

165. The method of any one of claims 126-164, further comprising cross-correlating the first order statistical analysis of the oculometric data.

166. The method of any one of claims 126-164, further comprising cross-correlating the first order statistical analysis of the facial biometrics data.

167. The method of any one of claims 1-166, further comprising performing a second order statistical analysis of the oculometric data and/or facial biometrics data.

168. The method of any one of claims 126-164, further comprising performing a higher order statistical analysis of the oculometric data and/or facial biometrics data.

169. The method of claim 168, wherein the higher order statistical analysis of the oculometric data and/or facial biometrics data comprises kurtosis.

170. The method of claim 169, further comprising determining the presence or absence of a change relative to baseline in the higher order statistical analysis of the oculometric data and/or facial biometrics data.

171. The method of claim 170, wherein the determining the presence or absence of a change relative to baseline in the higher order statistical analysis of the oculometric data and/or facial biometrics data comprises determining the presence of a change from frequency independence to inter-frequency dependence of the oculometric data and/or facial biometrics data.

172. The method of claim 170, wherein the determining the presence or absence of a change relative to baseline in the higher order statistical analysis of the oculometric data and/or facial biometrics data comprises determining change in synchronization of the oculometric data and/or facial biometrics data.

173. The method of claim 172, wherein the determining synchronization of the oculometric data and/or facial biometrics data comprises determining frequency synchronization of the oculometric data and/or facial biometrics data.

174. The method of claim 173, wherein the determining frequency synchronization comprises determining synchronization of dependent and/or uncoupled frequencies of the oculometric data and/or facial biometrics data.

175. The method of claim 170, wherein the determining the presence or absence of a change in the first order statistical analysis of the oculometric data and/or facial biometrics data comprises determining the presence of positive excess kurtosis of the oculometric data and/or facial biometrics data.

176. The method of claim 175, wherein the determining the presence of positive excess kurtosis of the oculometric data comprises determining the presence of positive excess kurtosis of eye eccentricity.

177. The method of claim 176, wherein the positive excess kurtosis is 10 or more.

178. The method of claim 176, wherein the positive excess kurtosis is 15 or more.

179. The method of any one of claims 126-178, wherein the determining step utilizes machine learning.

180. The method of any one of claims 126-179, further comprising cross-correlating the higher order statistical analysis of the oculometric data.

181. The method of any one of claims 126-179, further comprising cross-correlating the higher order statistical analysis of the facial biometrics data.

182. The method of any one of claims 126-179, further comprising measuring at least one electroencephalogram signal of the subject.

183. The method of claim 182, further comprising confirming the presence or absence of a change relative to baseline in the first order statistical analysis of the oculometric data using the at least one electroencephalogram signal.

184. The method of any one of claims 126-183, wherein the epileptic event in the subject is detected and/or predicted in the absence of measuring an electroencephalogram signal of the subject.

185. A method of detecting and/or predicting an epileptic event in a subject, said method comprising:
a) measuring left and right eye movements over time using a measuring device to obtain eye movement data from the subject;
b) identifying the presence or absence of an increase in the correlation of left and right eye movements over time based on the measuring; and
c) indicating that an epileptic seizure has been detected and/or predicted when the identifying indicates the presence of an increase in the correlation of left and right eye movements over time.

186. The method of claim 185, wherein the one or more eye movements comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.

187. The method of claim 185 or 186, wherein the measuring comprises measuring a change in two or more of the eye movements.

188. The method of any one of claims 185-187, wherein eye eccentricity is a function of visible x-width and y-width of the pupil of an eye.

189. The method of claim 188, wherein eye eccentricity changes as the eyelid position, position of the sides of the eye, pupil area, and/or blink frequency change(s).

190. The method of any one of claims 185-189, wherein the measuring device is configured to obtain eye movement data from the subject for about thirty minutes.

191. The method of any one of claims 185-189, wherein the measuring device is configured to obtain eye movement data from the subject for about fifteen minutes.

192. The method of claim 190 or 191, wherein the performing the first order statistical analysis of the eye movement data occurs in a ten-second running window.

193. The method of claim 190 or 191, wherein the performing the first order statistical analysis of the eye movement data occurs in a five-second running window.

194. The method of any one of claims 185-193, wherein the measuring device is an eye tracking device.

195. The method of claim 194, wherein the eye tracking device comprises one or more cameras.

196. The method of claim 195, wherein the eye tracking device further comprises a video recorder and/or a sensor.

197. The method of claim 196, wherein the eye tracking device is a wearable device configured to be worn on the head of the subject.

198. The method of claim 197, wherein the one or more cameras of the wearable device is located at a distance of one or more centimeters from the eyes of the subject.

199. The method of any one of claims 185-196, wherein the eye tracking device is a contact lens.

200. The method of any one of claims 185-199, wherein the eye movement data from the subject is captured at about 30 frames per second or more.

201. The method of any one of claims 185-199, wherein the eye movement data from the subject is captured at about 60 frames per second or more.

202. The method of any one of claims 185-199, wherein the eye movement data from the subject is captured at about 100 frames per second or more.

203. The method of any one of claims 185-199, wherein the eye movement data from the subject is captured at about 200 frames per second or more.

204. The method of any one of claims 185-203, further comprising measuring prodromal changes of the eye movement data.

205. The method of claim 204, wherein the prodromal changes occur one or more days before the epileptic event.

206. The method of claim 204, wherein the prodromal changes occur one or more hours before the epileptic event.

207. The method of claim 204, wherein the prodromal changes occur one or more seconds before the epileptic event.

208. The method of any one of claims 185-207, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.

209. The method of any one of claims 185-208, wherein the indicating that the epileptic event has been detected and/or predicted comprises providing an alert to the subject or a caregiver of the subject.

210. The method of any one of claims 185-209, further comprising providing a responsive neurostimulation to the subject, wherein the responsive neurostimulation is sufficient to reduce the effect of the epileptic event, when the epileptic event is detected and/or predicted.

211. The method of any one of claims 185-210, further comprising transmitting an electric current through the neck of the subject for which an epileptic event has been detected and/or predicted to a vagus nerve in the subject for which an epileptic event has been detected and/or predicted, wherein the electric current is sufficient to terminate the epileptic event, when the epileptic event is detected and/or predicted.

212. The method of any one of claims 185-211, further comprising administering an effective amount of an anti-epileptic drug to the subject, when the epileptic event is detected and/or predicted.

213. The method of claim 212, wherein the anti-epileptic drug comprises one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.

214. The method of any one of claims 185-213, further comprising cross-correlating eye movement data of a left eye and eye movement data of a right eye of the subject.

215. The method of any one of claims 185-214, further comprising measuring at least one electroencephalogram signal of the subject.

216. The method of claim 215, further comprising confirming the presence or absence of a change relative to baseline in the first order statistical analysis of the eye movement data using the at least one electroencephalogram signal.

217. The method of any one of claims 185-216, wherein the epileptic event in the subject is detected and/or predicted in the absence of measuring an electroencephalogram signal of the subject.

218. A system for detecting and/or predicting an epileptic event in a subject, the system comprising:
a) a measuring device configured to measure a change in one or more oculometric parameters of at least one eye of the subject over time;
b) a processor unit;
c) a non-transitory computer-readable storage medium comprising instructions, which when executed by the processor unit, cause the processor unit to perform a first order statistical analysis of the oculometric data and determine the presence or absence of a change relative to baseline in the first order statistical analysis of the oculometric data; and
c) an output device configured to indicate that an epileptic event has been detected and/or predicted when a change in the first order statistical analysis is determined to be present.

219. The system of claim 218, wherein the one or more oculometric parameters comprises eye eccentricity; pupil constriction rate; pupil constriction velocity; pupil dilation rate; pupil dilation velocity, hippus; eyelid movement rate; eyelid openings; eyelid closures; upward eyeball movements; downward eyeball movements; lateral eyeball movements; eye rolling; jerky eye movements; x and y location of pupil; pupil rotation; pupil area to iris area ratio; pupil diameter; saccadic velocity; torsional velocity; saccadic direction; torsional direction; eye blink rate; eye blink duration; and/or eye activity during sleep.

220. The system of claim 218 or 219, wherein the measuring device measures a change in two or more of the oculometric parameters.

221. The system of any one of claims 218-220, wherein eye eccentricity is a function of visible x-width and y-width of the pupil of an eye.

222. The system of claim 221, wherein eye eccentricity changes as the eyelid position, position of the sides of the eye, pupil area, and/or blink frequency change(s).

223. The system of any one of claims 218-222, wherein the first order statistical analysis of the oculometric data comprises multiple regression analysis and/or mean calculations of the oculometric data.

224. The system of any one of claims 218-223, wherein the measuring device is configured to obtain oculometric data from the subject for about thirty minutes.

225. The system of any one of claims 218-223, wherein the measuring device is configured to obtain oculometric data from the subject for about fifteen minutes.

226. The system of claim 224 or 225, wherein the non-transitory computer-readable storage medium comprises instructions, which when executed by the processor unit, cause the processor unit to perform the first order statistical analysis of the oculometric data in a ten-second running window.

227. The system of claim 224 or 225, wherein the non-transitory computer-readable storage medium comprises instructions, which when executed by the processor unit, cause the processor unit to perform the first order statistical analysis of the oculometric data in a five-second running window.

228. The system of any one of claims 218-227, wherein the measuring device is an eye tracking device.

229. The system of claim 228, wherein the eye tracking device comprises one or more cameras.

230. The system of claim 229, wherein the eye tracking device further comprises a video recorder and/or a sensor.

231. The system of claim 230, wherein the eye tracking device is a wearable device configured to be worn on the head of the subject.

232. The system of claim 231, wherein the one or more cameras of the wearable device is located at a distance of one or more centimeters from the eyes of the subject.

233. The system of any one of claims 218-230, wherein the eye tracking device is a contact lens.

234. The system of any one of claims 218-233, wherein the non-transitory computer-readable storage medium comprising instructions, which when executed by the processor unit, cause the processor unit to perform the first order statistical analysis of the oculometric data comprises performing multiple regression analysis of the oculometric data.

235. The system of claim 234, wherein determining the presence or absence of a change in the first order statistical analysis of the oculometric data comprises determining the presence or absence of an increased correlation of one or more oculometric parameters with the epileptic event.

236. The system of claim 235, wherein determining the presence or absence of an increased correlation of one or more oculometric parameters with the epileptic event comprises determining the presence or absence of an increased correlation of eye eccentricity with the epileptic event.

237. The system of any one of claims 218-236, wherein the oculometric data from the subject is captured at about 30 frames per second or more.

238. The system of any one of claims 218-236, wherein the oculometric data from the subject is captured at about 60 frames per second or more.

239. The system of any one of claims 218-236, wherein the oculometric data from the subject is captured at about 100 frames per second or more.

240. The system of any one of claims 218-236, wherein the oculometric data from the subject is captured at about 200 frames per second or more.

241. The system of any one of claims 218-240, wherein the measuring device is further configured to measure a change in one or more facial biometrics of the subject to provide facial biometrics data.

242. The system of claim 241, wherein the non-transitory computer readable storage medium further comprises instructions, which when executed by the processor unit, cause the processor unit to perform a first order statistical analysis of the facial biometrics data.

243. The system of claim 242, wherein the non-transitory computer readable storage medium further comprises instructions, which when executed by the processor unit, cause the processor unit to determine the presence or absence of a change relative to baseline in the first order statistical analysis of the facial biometrics data.

244. The system of any one of claims 241-243, wherein the one or more facial biometrics comprises distance between the eyes; distance between the eyelids; width of the nose; center of the nose; depth of the eye sockets; shape of the cheekbones; length of the jawline; distance between the mouth edges; center of the mouth; and/or focal weakness.

245. The system of any one of claims 218-244, wherein the measuring device is further configured to measure prodromal changes of the oculometric data and/or facial biometrics data.

246. The system of claim 245, wherein the prodromal changes occur one or more days before the epileptic event.

247. The system of claim 245, wherein the prodromal changes occur one or more hours before the epileptic event.

248. The system of claim 245, wherein the prodromal changes occur one or more seconds before the epileptic event.

249. The system of any one of claims 245-248, wherein the non-transitory computer readable storage medium further comprises instructions, which when executed by the processor unit, cause the processor unit to perform a first order statistical analysis of the prodromal changes of the oculometric data and/or facial biometrics data.

250. The system of claim 249, wherein the non-transitory computer readable storage medium further comprises instructions, which when executed by the processor unit, cause the processor unit to determine the presence or absence of a change relative to baseline in the first order statistical analysis of the prodromal changes of the oculometric data and/or facial biometrics data.

251. The system of any one of claims 218-250, wherein the epileptic event comprises a partial seizure, a myoclonic seizure, an infantile spasm, a tonic seizure, an atonic seizure, a frontal lobe seizure, Todd's paralysis, and/or sudden unexpected death in epilepsy.

252. The system of any one of claims 218-251, wherein the output device configured to indicate that the epileptic event has been detected and/or predicted comprises providing an alert to the subject or a caregiver of the subject.

253. The system of claim any one of claims 218-252, further comprising a neurostimulation device configured to provide a responsive neurostimulation to the subject, wherein the responsive neurostimulation is sufficient to reduce the effect of the epileptic event, when the epileptic event is detected and/or predicted.

254. The system of claim any one of claims 218-253, further comprising a neurostimulation device configured to provide an electric current through the neck of the subject for which an epileptic event has been detected and/or predicted to a vagus nerve in the subject for which an epileptic event has been detected and/or predicted, wherein the electric current is sufficient to terminate the epileptic event, when the epileptic event is detected and/or predicted.

255. The system of any one of claims 218-254, further comprising a drug administration device configured to administer an effective amount of an anti-epileptic drug to the subject, when the epileptic event is detected and/or predicted.

256. The system of claim 255, wherein the anti-epileptic drug comprises one or more of intravenous lorazepam; acetazolamide; carbamazepine; clobazam; clonazepam; eslicarbazepine acetate; ethosuximide; gabapentin; lacosamide; lamotrigine; levetiracetam; nitrazepam; oxcarbazepine; perampanel; piracetam; phenobarbital; phenytoin; pregabalin; primidone; rufinamide; sodium valproate; stiripentol; tiagabine; topiramate; vigabatrin; and zonisamide.

257. The system of any one of claims 218-256, wherein measuring a change in one or more oculometric parameters of at least one eye comprises measuring a change in one or more oculometric parameters of both the left eye and the right eye.

258. The system of claim 257, wherein the one or more oculometric parameters comprise left and right eye movements.

259. The system of claim 257 or 258, wherein the non-transitory computer-readable storage medium comprises instructions, which when executed by the processor unit, cause the processor unit to cross-correlate oculometric data of a left eye and oculometric data of a right eye of the subject.

260. The system of any one of claims 257-259, wherein determining the presence or absence of the change relative to baseline in the first order statistical analysis of the oculometric data comprises determining the presence of an increase in the synchronization of eye movements between the left eye and the right eye of the subject relative to baseline.

261. The system of any one of claims 218-260, wherein the non-transitory computer-readable storage medium comprises instructions, which when executed by the processor unit, cause the processor unit to cross-correlate the first order statistical analysis of the oculometric data.

262. The system of any one of claims 218-260, wherein the non-transitory computer-readable storage medium comprises instructions, which when executed by the processor unit, cause the processor unit to cross-correlate the first order statistical analysis of the facial biometrics data.

263. The system of any one of claims 218-262, wherein the non-transitory computer-readable storage medium comprises instructions, which when executed by the processor unit, cause the processor unit to perform a second order statistical analysis of the oculometric data and/or facial biometrics data.

264. The system of any one of claims 218-260, wherein the non-transitory computer readable storage medium further comprises instructions, which when executed by the processor unit, cause the processor unit to perform a higher order statistical analysis of the oculometric data and/or facial biometrics data.

265. The system of claim 264, wherein the higher order statistical analysis of the oculometric data and/or facial biometrics data comprises kurtosis.

266. The system of claim 265, wherein the non-transitory computer readable storage medium further comprises instructions, which when executed by the processor unit, cause the processor unit to determine the presence or absence of a change relative to baseline in the higher order statistical analysis of the oculometric data and/or facial biometrics data.

267. The system of claim 266, wherein determining the presence or absence of a change relative to baseline in the higher order statistical analysis of the oculometric data and/or facial biometrics data comprises determining the presence of a change from frequency independence to inter-frequency dependence of the oculometric data and/or facial biometrics data.

268. The system of claim 266, wherein determining the presence or absence of a change relative to baseline in the higher order statistical analysis of the oculometric data and/or facial biometrics data comprises determining change in synchronization of the oculometric data and/or facial biometrics data.

269. The system of claim 268, wherein determining synchronization of the oculometric data and/or facial biometrics data comprises determining frequency synchronization of the oculometric data and/or facial biometrics data.

270. The system of claim 269, wherein determining frequency synchronization comprises determining synchronization of dependent and/or uncoupled frequencies of the oculometric data and/or facial biometrics data.

271. The system of claim 266, wherein determining the presence or absence of a change in the first order statistical analysis of the oculometric data and/or facial biometrics data comprises determining the presence of positive excess kurtosis of the oculometric data and/or facial biometrics data.

272. The system of claim 271, wherein determining the presence of positive excess kurtosis of the oculometric data comprises determining the presence of positive excess kurtosis of eye eccentricity.

273. The system of claim 272, wherein the positive excess kurtosis is 10 or more.

274. The system of claim 272, wherein the positive excess kurtosis is 15 or more.

275. The system of any one of claims 218-274, wherein the system is aided by machine learning.

276. The system of any one of claims 218-275, wherein the non-transitory computer-readable storage medium comprising instructions, which when executed by the processor unit, cause the processor unit to cross-correlate the higher order statistical analysis of the oculometric data.

277. The system of any one of claims 218-275, wherein the non-transitory computer-readable storage medium comprises instructions, which when executed by the processor unit, cause the processor unit to cross-correlate the higher order statistical analysis of the facial biometrics data.

278. The system of any one of claims 218-275, wherein the processor unit comprises a memory field for containing a computer interface.

279. The system of any one of claims 218-278, wherein the output device comprises a memory field for containing a computer interface.

280. The system of any one of claims 218-279, further comprising an input device configured to measure at least one electroencephalogram signal on the subject.

281. The system of claim 280, wherein the non-transitory computer-readable storage medium comprises instructions, which when executed by the processor unit, cause the processor unit to confirm the presence or absence of a change relative to baseline in the first order statistical analysis of the oculometric data using the at least one electroencephalogram signal.

282. The system of any one of claims 218-281, wherein the epileptic event in the subject is detected and/or predicted in the absence of measuring an electroencephalogram signal of the subject.
